(19) United States
(12) Patent Application Publication
(10) Pub. No.: US 2023/0151100 A1
(43) Pub. Date: May 18, 2023

(54) Method of Selecting Patients for Treatment with a Combination of an AXL Inhibitor and an Immune Checkpoint Modulator

(71) Applicants: BERGENBIO ASA, Bergen (NO); THE BOARD OF REGENTS OF THE UNIVERSITY OF TEXAS SYSTEM, Austin, TX (US)

(72) Inventors: James LORENS, Bergen (NO); David MICKLEM, Bergen (NO); Huiyu LI, Dallas, TX (US); Zhida LIU, Taiyuan City (CN); John MINNA, Dallas, TX (US); Rolf BREKKEN, Dallas, TX (US)

(73) Assignees: BERGENBIO ASA, Bergen (NO); THE BOARD OF REGENTS OF THE UNIVERSITY OF TEXAS SYSTEM, Austin, TX (US)

(21) Appl. No.: 17/920,442

(22) PCT Filed: Apr. 23, 2021
(86) PCT No.: PCT/GB2021/050999

§ 371 Date: Oct. 21, 2022

Related U.S. Application Data
(60) Provisional application No. 63/118,764, filed on Nov. 27, 2020.
(60) Provisional application No. 63/144,296, filed on Feb. 1, 2021.
(60) Provisional application No. 63/152,654, filed on Feb. 23, 2021.

(30) Foreign Application Priority Data
Apr. 24, 2020 (GB) 2006072.9

(51) Int. Cl. C07K 16/28; A61K 45/06; A61P 35/00

(52) U.S. Cl. CPC C07K 16/2863; A61K 45/06; A61P 35/00; A61K 2039/505

(57) ABSTRACT

The invention relates to a method of selecting a subject for treatment with a combination of an AXL inhibitor (AXLi) and an immune checkpoint modulator (ICM), the method comprising: identifying subjects having an AXL-related disease characterised by the presence of cells having modified STK11 activity or expression; and, selecting thus identified subjects for treatment. The invention also relates to an AXL inhibitor (AXLi) and an immune checkpoint modulator (ICM) for use in the treatment of an AXL-related disease.

This disclosure relates to methods of selecting subjects for treatment with a combination therapy which comprises an AXL-kinase inhibitor and an immune-regulatory agent. The disclosure also relates to methods of treating subjects selected by such methods, and methods of treating a defined subgroup of subjects having neoplastic disorders, with a combination therapy which comprises an AXL-kinase inhibitor and an immune-regulatory agent.

### BACKGROUND

AXL

All of the protein kinases that have been identified to date in the human genome share a highly conserved catalytic domain of around 300 amino acids. This domain folds into a bi-lobed structure in which resides ATP-binding and catalytic sites. The complexity of protein kinase regulation allows many potential mechanisms of inhibition including competition with activating ligands, modulation of positive and negative regulators, interference with protein dimerization, and allosteric or competitive inhibition at the substrate or ATP binding sites.

AXL (also known as UFO, ARK, and Tyro7; nucleotide accession numbers NM_021913 and NM_001699; protein accession numbers NP_068713 and NP_001690) is a receptor protein tyrosine kinase (RTK) that comprises a C-terminal extracellular ligand binding domain and N-terminal cytoplasmic region containing the catalytic domain. The extracellular domain of AXL has a unique structure that juxtaposes immunoglobulin and fibronectin Type III repeats and is reminiscent of the structure of neural cell adhesion molecules. AXL and its two close relatives, Mer/Nyk and Sky (Tyro3/Rse/Dtk), collectively known as the Tyro3 family of RTK's, all bind and are stimulated to varying degrees by the same ligand, GAS6 (growth arrest specific-6), a ˜76 kDa secreted protein with significant homology to the coagulation cascade regulator, Protein S. In addition to binding to ligands, the AXL extracellular domain has been shown to undergo homophilic interactions that mediate cell aggregation, suggesting that one important function of AXL may be to mediate cell-cell adhesion.

AXL is predominantly expressed in the vasculature in both endothelial cells (EC's) and vascular smooth muscle cells (VSMC's) and in cells of the myeloid lineage and is also detected in breast epithelial cells, chondrocytes, Sertoli cells and neurons. Several functions including protection from apoptosis induced by serum starvation, TNF-α or the viral protein E1A, as well as migration and cell differentiation have been ascribed to AXL signalling in cell culture. AXL has been found to serve as a key checkpoint for interferon (IFN) signalling (Rothlin et al, 2007; Huang et al, 2015); in the context of viral responses, the Zika virus has been found to antagonize the IFN action by interacting with AXL (Chen et al, 2018). However, Axl−/− mice exhibit no overt developmental phenotype and the physiological function of AXL in vivo is not clearly established in the literature.

AXL Pathology

The overexpression of AXL and/or its ligand has also been reported in a wide variety of solid tumor types including, but not limited to, breast, renal, endometrial, ovarian, thyroid, non-small cell lung carcinoma, and uveal melanoma as well as in myeloid leukemias. Furthermore, it possesses transforming activity in NIH3T3 and 32D cells. It has been demonstrated that loss of Axl expression in tumor cells blocks the growth of solid human neoplasms in an in vivo MDA-MB-231 breast carcinoma xenograft model. Taken together, these data suggest AXL signalling can independently regulate EC angiogenesis and tumor growth and thus represents a novel target class for tumor therapeutic development.

The expression of AXL and GAS6 proteins is upregulated in a variety of other disease states including endometriosis, vascular injury and kidney disease and AXL signalling is functionally implicated in the latter two indications. AXL-GAS6 signalling amplifies platelet responses and is implicated in thrombus formation. AXL may thus potentially represent a therapeutic target for a number of diverse pathological conditions including solid tumors, including, but not limited to, breast, renal, endometrial, ovarian, thyroid, non-small cell lung carcinoma and uveal melanoma; liquid tumors, including but not limited to, leukemias (particularly myeloid leukemias) and lymphomas; endometriosis, vascular disease/injury (including but not limited to restenosis, atherosclerosis and thrombosis), psoriasis; visual impairment due to macular degeneration; diabetic retinopathy and retinopathy of prematurity; kidney disease (including but not limited to glomerulonephritis, diabetic nephropathy and renal transplant rejection), rheumatoid arthritis; osteoporosis, osteoarthritis and cataracts.

AXL Inhibitors

In view of the role played by AXL in numerous pathological conditions, the development of safe and effective AXL inhibitors has been a topic of interest in recent years. Different groups of AXL inhibitors are discussed in, inter alia, US20070213375, US 20080153815, US20080188454, US20080176847, US20080188455, US20080182862, US20080188474, US20080117789, US20090111816, WO2007/0030680, WO2008/045978, WO2008/083353, WO2008/0083357, WO2008/083354, WO2008/083356, WO2008/080134, WO2009/054864, and WO/2008/083367.

Combination Therapies Using AXL Inhibitors

The combination of one or more of the above cited AXL inhibitors with one or more other agents is discussed in, for example, WO/2010/083465 and WO/2016/193680, with WO/2016/193680 focussing on combinations of AXL inhibitors with agents having immune-regulatory or modulatory activity. For example, inhibition of AXL with the small molecule Bemcentinib (BGB324/R428) was found to enhance the efficacy of immune checkpoint inhibitor treatment with anti PD1 and/or anti CTLA4.

Combination Therapies Using Immune Checkpoint Modulators

It is increasingly recognized that the effectiveness of conventional cytotoxic chemotherapeutic treatments is at least partially mediated through its interplay with the tumor and host immune response. The different classes of cytotoxic drugs have specific effects on the immune contexture, with varying ability to induce immunogenic cell death and influence suppressive and effector immune cells (Galluzzi et al, 2015; Yan et al, 2018). The combination of inhibition of the PD1/PDL1-axis with cytotoxic chemotherapy is currently being explored in several clinical trials, including a phase 3 clinical trial in triple negative breast cancer (TNBC) (Emens et al, 2016), with a reported increased median overall survival as compared to chemotherapy alone in a phase 3 clinical trial in non-small cell lung cancer (NSCLC) (Gandhi et al, 2018).

KRAS

The KRAS (Kirsten rat sarcoma 2 viral oncogene homolog) gene is a proto-oncogene that encodes a small GTPase transductor protein called KRAS, part of the RAS/MAPK (MAPK/ERK) pathway. KRAS has a role in the regulation of cell division as a result of its ability to relay external signals to the cell nucleus. Activating mutations in the KRAS gene are known to impair ability of the KRAS protein to switch between active and inactive states, resulting in constitutive activation of the protein and downstream signalling cascades, and thus uncontrolled cell proliferation and survival. KRAS mutations are found at high rates in many cancers, and are associated with increased resistance to chemotherapy and biological therapies targeting epidermal growth factor receptors (Yang et al, 2019; Jancik et al, 2010). KRAS mutations are the most prevalent oncogenic driver in NSCLC, accounting for approximately 25% of lung adenocarcinoma (Skoulidis et al, 2018).

STK11

Serine/threonine kinase 11 (STK11; also known as: liver kinase Bl, LKB1; renal carcinoma antigen NY-REN-19) is a protein kinase that in humans is encoded by the STK11 gene. STK11 is known to regulate cellular energy homeostasis, growth, and cell polarity through phosphorylation of adenosine monophosphate-activated protein kinase (AMPK), an enzyme with a role in cellular energy homeostasis, and 12 AMPK-related kinases. STK11 activates AMPK, resulting in suppression of growth and proliferation when energy and nutrient levels are scarce. STK11 activation of AMPK-related kinases plays a vital role in maintaining cell polarity thereby inhibiting inappropriate expansion of tumour cells (Shackelford et al, 2009). STK11 is known to function as a tumour suppressor, with STK11 mutations associated with numerous cancers (Zhao & Xu, 2014). STK11 alterations have been identified as the most prevalent genomic driver of primary resistance to PD-1 axis inhibitors in KRAS -mutant lung adenocarcinoma, as well as in PD-L1—positive NSCLC regardless of KRAS status, and patients with KRAS-mutant NSCLC (Skoulidis et al, 2018). STK11IP (STK11 interacting protein; serine/threonine kinase 11 interacting protein) is a known interaction partner of STK11 (Moren et al, 2011). It has been shown to bind directly to STK11 and affect subcellular localisation of STK11 (sequestering it in the cytoplasm rather than the nucleus).

P53

p53 (also known as: tumor protein p53; cellular tumor antigen; the Guardian of the Genome; phosphoprotein p53; tumor suppressor p53; antigen NY-CO-13; transformation-related protein 53, TRP53), is any isoform of a protein encoded by homologous genes in various organisms, such as TP53 in humans and Trp53 in mice. In humans, the TP53 gene is the most frequently mutated gene (>50%) in cancer, indicating that the TP53 gene plays a crucial role in preventing cancer formation. p53 plays a role in regulation or progression through the cell cycle, apoptosis, and genomic stability by several mechanisms including activation of DNA repair, arresting cell growth, and initiating apoptosis. If the TP53 gene is damaged, tumour suppression is severely compromised (Joerger & Fersht, 2016).

Stem-Like T Cell Factor 1

Stem-like T cell factor 1 (TCF1, encoded by Tcf7) expressing PD-1⁺ CD8⁺ T cells are a key cell population that respond to the PD-1/PD-L1 blockade. The expansion of differentiated TCF1⁻ PD-1⁺ CD8⁺ T cells in response to anti-PD-1/PD-L1 therapy leads to control of tumor cell growth. The origins of TCF1⁺ T cells in the TME are unclear and no pharmacological agent has been shown to expand TCF1⁺ PD-1⁺ CD8⁺ T cells effectively.

Although anti-PD-1/PD-L1 treatment can reverse the exhaustion status of CD8⁺ T cells in tumors, the activation and differentiation of tumor specific CD8⁺ T cells requires antigen presentation by antigen presenting cells (APCs). Axl, a receptor tyrosine kinase, is an innate checkpoint in regulating APC activation. Activation of Axl on innate immune cells suppresses type I interferon production, a critical signaling pathway for priming and activating anti-tumor associated CD8⁺ T cells.

The complexity of tumour biology and its interaction with the immune system, along with the potential for serious side-effects inherent in such powerful therapies, means that research is ongoing to identify efficacious combination therapies, and specific disorders and/or subjects that will benefit most from such treatments.

### SUMMARY

The present disclosure is based on the finding that combination therapies comprising an AXL inhibitor (such as Bemcentinib, BGB324) and an immune checkpoint modulator (such as the PD1 inhibitor Pembrolizumab) are effective in non-small cell lung cancer patients, including in patients with STK11 mutations. This was unexpected in view of literature reports that subjects with STK11 mutations are resistant to treatment with PD1 inhibitors (see, e.g., Skoulidis et al, 2018).

Accordingly, in a first aspect the present disclosure provides a method of selecting a subject for treatment with a combination of an AXL inhibitor (AXLi) and an immune checkpoint modulator (ICM), the method comprising: identifying subjects having an AXL-related disease characterised by the presence of cells having modified STK11 activity or expression; and, selecting thus identified subjects for treatment.

In a second aspect the present disclosure also provides a method of selecting a subject for treatment with a combination of an AXL inhibitor (AXLi) and an immune checkpoint modulator (ICM), the method comprising: identifying subjects that have previously been treated with an immune checkpoint modulator (ICM) and which did not respond to or benefit from treatment with the ICM; and, selecting thus identified subjects for treatment.

In a third aspect the present disclosure also provides a method of selecting a subject for treatment with a combination of an AXL inhibitor (AXLi) and an immune checkpoint modulator (ICM), the method comprising: identifying subjects having an AXL-related disease characterised by a reduced presence of CD8⁺ cells having TCF1 activity or expression; and, selecting thus identified subjects for treatment.

The treatment for which the subject is selected may comprise a combination of an AXL inhibitor (AXLi), an immune checkpoint modulator (ICM), and a chemotherapeutic agent and/or radiotherapy.

The methods may comprise identifying subjects wherein the AXL-related disease is further characterised by: i) the presence of cells having increased KRAS activity or expression; ii) the presence of cells having decreased p53 activity or expression; and/or; iii) the presence of cells having increased AXL activity or expression.

Increased or decreased expression may be assessed by: determining copy number of the gene encoding STK11, KRAS, or p53 relative to a control sample; and/or determining the level of STK11, KRAS, or p53 protein or mRNA relative to a control sample. Modified STK11 activity or expression may be assessed by determining the presence or absence of a STK11 mutation and/or a STK11IP mutation.

Increased or reduced presence of CD8+ cells having TCF1 activity or expression may be assessed by comparing the number of CD8+ cells having TCF1 activity or expression relative to a control sample.

The STK11 mutation or STK11IP mutation may be: a mutation in the nucleotide sequence encoding STK11 or STK11IP; a mutation in a regulatory sequence controlling expression of the nucleotide sequence encoding STK11 or STK11IP; a mutation in a nucleotide encoding a protein which interacts with the transcription product of the STK11 or STK11IP gene; a mutation in the translation product of the STK11 or STK11IP gene; and/or a mutation in the transcription product of the STK11 or STK11IP gene. The STK11 mutation may be an inactivating mutation, and/or the STK11IP mutation may be an activating mutation. Increased or decreased activity or expression may be determined in a sample derived from a subject. Increased or decreased activity or expression may be determined relative to a control.

The AXL-related disease may be a proliferative disease, a solid tumour, or cancer. The cancer may be selected from the group consisting of: consisting of: lung cancer, non-small-cell lung cancer, breast cancer, melanoma, mesothelioma, acute myeloid leukemia (AML), myelodysplastic syndrome (MDS), pancreas cancer, kidney cancer, urothelial carcinoma, and glioblastoma. The cancer may be lung cancer, preferably non-small-cell lung cancer.

The methods may further comprise administering to the subject a therapeutically effective amount of an AXL inhibitor (AXLi), an immune checkpoint modulator (ICM), and/or a chemotherapeutic agent and/or radiotherapy.

The AXL inhibitor may be a compound of formula (I) as described in more detail elsewhere herein:

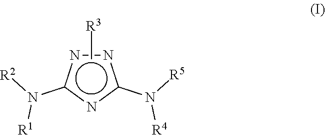

The AXL inhibitor may be Bemcentinib. The AXL inhibitor may also be an antibody; for example, an antibody comprising the 6 CDRs having the sequences of SEQ ID Nos. 1 to 6, or the 6 CDRs having the sequences of SEQ ID Nos. 7 to 12.

The immune checkpoint modulator (ICM) may be an immune checkpoint inhibitor (ICI), or a T cell co-stimulatory agonist. For example, the ICM may be an immune checkpoint modulating antibody selected from the group consisting of: anti-CTLA-4 antibodies, anti-PD-1 antibodies, anti-PD-L1 antibodies, anti-4-1 BB antibodies, anti-OX-40 antibodies, anti-GITR antibodies, anti-CD27 antibodies, anti-CD28 antibodies, anti-CD40 antibodies, anti-LAGS antibodies, anti-ICOS antibodies, anti-TWEAKR antibodies, anti-HVEM antibodies, anti-TIM-1 antibodies, anti-TIM-3 antibodies, anti-VISTA antibodies, and anti-TIGIT antibodies. The immune checkpoint modulator (ICM) may be selected from the group consisting of: anti-CTLA-4 antibodies, anti-PD-1 antibodies, and anti-PD-L1 antibodies. The immune checkpoint modulator may include, or may be: pembrolizumab; ipilimumab; ipilimumab and nivolumab; ipilimumab and pembrolizumab; tremelilumab and durvalumab.

The chemotherapeutic agent may be a chemotherapeutic agent which induces immunogenic cell death of cancer cells and/or which induces an immune response in the subject. The chemotherapeutic agent may be a chemotherapeutic agent which induces a type I interferon response in the subject. The chemotherapeutic agent may be an anthracycline, for example, doxorubicin, daunorubicin, epirubicin, idarubicin, mitoxantrone, or valrubicin. The chemotherapeutic agent may be doxorubicin.

In a fourth aspect the present disclosure provides a method of treating an AXL-related disease in a subject with a combination of an AXL inhibitor (AXLi) and an immune checkpoint modulator (ICM), wherein the AXL-related disease is characterised by: the presence of cells having modified STK11 activity or expression; and/or the presence of cells having a STK11 mutation and/or a STK11IP mutation. The method may comprise: (i) administering a combination of the AXLi and the ICM to the subject; (ii) administering the AXLi to the subject, wherein the ICM has been, is, or will be, administered to the subject; or (iii) administering the ICM to the subject, wherein the AXLi has been, is, or will be, administered to the subject.

The subject may be treated with a combination of an AXL inhibitor (AXLi), an immune checkpoint modulator (ICM), and a chemotherapeutic agent and/or radiotherapy. The subject may have been selected for treatment using a method according to the first, second or third aspect of the disclosure.

In a fifth aspect, the present disclosure provides a reagent for detecting activity, expression, or amount of STK11, or STK11IP for use in a method according to the first or second aspect of the disclosure. Also provided is a kit comprising 1, 2, 3, 4, or more reagents for detecting activity, expression, or amount of one or more of STK11, STK11IP, KRAS, or p53, for use in a method according to the first or second aspect of the disclosure.

In addition, included in the fifth aspect are: an AXL inhibitor, an immune checkpoint modulator (ICM), and/or a chemotherapeutic agent for use in a method according to the fourth aspect.

In a sixth aspect, the disclosure provides a method of prognosing susceptibility of a subject to treatment with a combination of an AXL inhibitor (AXLi) and an immune checkpoint modulator (ICM), the method comprising: determining: i) the presence or absence of a STK11 mutation and/or STK11IP mutation; and/or ii) the level of STK11 activity or expression in the subject or a sample derived from the subject;

wherein the presence of a STK11 mutation and/or presence of a STK11IP mutation, and/or a decreased level of STK11 activity or expression is indicative of susceptibility to treatment with a combination of an AXL inhibitor (AXLi) and an immune checkpoint modulator (ICM).

In a seventh aspect, the disclosure provides a method of increasing a population of desired T cells in a subject comprising treating the subject with an AXL inhibitor.

In embodiments, the desired T cells may be TCF1⁺ T cells, PD-1⁺ T cells, CD8⁺ T cells, TCF1⁺ PD-1⁺ T cells, or TCF1⁺ PD-1⁺ CD8⁺ T cells, or T cells having any other combination of these markers.

In an eighth aspect, the disclosure provides a method of treating an AXL-related disease in a subject in need of such treatment, the method comprising administering to the subject a combination of an AXL inhibitor (AXLi) and an immune checkpoint modulator (ICM), wherein the AXLi and ICM may be administered to the subject simultaneously, separately or sequentially. The AXL-related disease may be a neoplastic disease, such as cancer (for example as described herein). The ICM may, in exemplary embodiments, be an anti-PD-1 or anti-PD-L1 agent. Similarly, the disclosure provides an AXL inhibitor (AXLi) and an immune checkpoint modulator (ICM) for use in the treatment of an AXL-related disease in a subject, wherein the AXLi and ICM may be administered to the subject simultaneously, separately or sequentially.

In certain embodiments of the eighth aspect, the AXLi is used in combination with a single ICM.

The disclosure includes the combination of the aspects and preferred features described except where such a combination is clearly impermissible or expressly avoided.

### DETAILED DESCRIPTION

Aspects and embodiments of the present disclosure will now be discussed with reference to the accompanying figures. Further aspects and embodiments will be apparent to those skilled in the art. All documents mentioned in this text are incorporated herein by reference.

The present disclosure pertains to methods of selecting subjects for treatment with a combination of an AXL-kinase inhibitor and an immune checkpoint modulator, and to methods of treating subjects selected by such methods with a combination of an AXL-kinase inhibitor and an immune checkpoint modulator. More particularly, the present disclosure pertains to methods of selecting patients for treatment with a combination of an AXL-kinase inhibitor and an immune checkpoint modulator by identifying subjects having an AXL-related disease characterised by the presence of cells having altered activity or expression of one or more proteins of interest, and selecting thus identified subjects for treatment.

There is a severe unmet medical need for treatment options for patients who have previously been treated with a monotherapy or combination therapies but have had disease progression. Standard mono and combination therapies include combinations of monoclonal antibodies with other antibodies, or with other chemotherapeutic agents. For example Pembrolizumab in combination with pemetrexed and platinum chemotherapy, or Nivolumab in combination with Ipilimumab.

In a Phase II clinical study investigating the efficacy of a combination therapy including an AXL kinase inhibitor (Bemcentinib; BGB324) and an immune checkpoint inhibitor (anti-PD-1; Pembrolizumab) against non-small cell lung cancer, the present inventors observed anti-tumor effects and increases in progression free survival in a number of “responder” subjects. Subsequent whole exome sequence analysis of biopsies collected from responder patients identified mutations in STK11, or the gene encoding the STK11 associated protein STK11IP (serine/threonine kinase 11 interacting protein), in three responder subjects. This was unexpected in view of the literature reports of subjects with STK11 mutations responding poorly to PD-1 inhibition.

The pronounced response of the STK11 subject sub-group was particularly unexpected in view of the prevailing understanding of the effect on STK11 mutations on the efficacy of treatment with PD-1 inhibitors, with mutated STK11, KRAS, and p53 reported as defining distinct patient subgroups in lung adenocarcinoma that respond poorly to PD-1 inhibitors. For example Skoulidis et al (2018, 2019a, 2019b) have reported genomic alterations in STK11 as a tumor cell—intrinsic determinant of primary resistance to PD-1 axis blockade in three independent retrospective cohorts of KRAS-mutant lung adenocarcinoma. The impact of STK11 alterations on clinical outcome with PD-1 inhibitors was also reported to extend to PD-L1-positive non-small cell lung cancer.

The authors' observation of PD-1 blockade efficacy in the STK11 subject sub-group therefore ran counter to the accepted teaching in the field and demonstrated a role for the co-administration of AXL kinase inhibitors in restoring and enhancing the efficacy of PD-1 blockade in these subject sub-groups. The involvement of AXL in STK11/PD-1 is a new finding, with no suggestion of a role for AXL or AXL-related processes such as EMT suggested in earlier publications concerning STK11 and PD-1 blockade.

Further analysis of clinical trial data by the present authors has confirmed AXL upregulation in STK11 mutated tumours. The present authors therefore infer that AXL plays a role in the mechanism of resistance to anti-PD-1 checkpoint modulation in STK11 mutant subjects, with inhibition of AXL a means of overcoming this resistance.

The Laboratories of Drs. John D. Minna and Rolf A. Brekken at the University Texas Southwestern Medical Center presented data demonstrating that tumors comprising Kras^{G12D}, p53^{-/-} STK11^{-/-} mutant (KPL9-3) mouse lung-cancer derived cells were resistant to anti-PD1 therapy and AXL inhibitors as monotherapies, but were sensitive to combination treatments comprising an immune checkpoint inhibitor and an AXL inhibitor. The contents of this presentation are incorporated herein by reference in their entirety. Furthermore, an Axl inhibitor sensitizes STK11-mutant human NSCLC Kras^{G12D}, p53 WT STK11^{-/-} tumors in humanized mice to treatment with anti-PD1.

The present inventors also systematically evaluated CD8⁺ T cells status changes in tumor microenvironment (TME) mediated by KL mutation, allowing them to find the key factor for PD-1/PD-L1 resistance in KL mutated tumors. They also showed that the growth pattern differences of KL mutated tumors were likely mediated by immune system. This proved that it is possible to sensitize KL mutated NSCLC patients to PD-1/PD-L1 treatment through Axl inhibition with BGB324. These results also show that it is possible to provide ways to expand TCF1⁺PD-1⁺CD8⁺ T cells in TME.

Accordingly, the present disclosure provides a method of selecting a subject for treatment with a combination of an AXL inhibitor (AXLi) and an immune checkpoint modulator (ICM), wherein the method comprises: identifying subjects having an AXL-related disease characterised by the presence of cells having modified STK11 activity or expression; and, selecting thus identified subjects for treatment.

The present disclosure also provides a reagent for detecting activity, expression, or amount of STK11, for use in a method of selecting a subject for treatment with a combination of an AXL inhibitor (AXLi) and an immune checkpoint modulator (ICM). The method of selecting may comprise: identifying subjects having an AXL-related disease characterised by the presence of cells having modified STK11 activity or expression; and, selecting thus identified subjects for treatment.

The present disclosure also provides a method of treating an AXL-related disease in a subject with a combination of an AXL inhibitor (AXLi) and an immune checkpoint modulator (ICM), the method comprising: administering a combination of the AXLi and the ICM to the subject; administering the AXLi to the subject, wherein the ICM has been, is, or will be, administered to the subject; or, administering the ICM to the subject, wherein the AXLi has been, is, or will be, administered to the subject; wherein the subject has been selected for treatment using a method of selecting a subject as disclosed herein.

The present disclosure also provides a method of selecting a subject for treatment with a combination of an AXL inhibitor (AXLi) and an immune checkpoint modulator (ICM), the method comprising: identifying subjects having an AXL-related disease characterised by a reduced presence of CD8⁺ cells having TCF1 activity or expression; and, selecting thus identified subjects for treatment.

The present disclosure also provides a method of increasing a population of desired T cells in a subject comprising treating the subject with an AXL inhibitor (AXLi).

Methods of Selecting a Subject for Treatment

The methods of the disclosure include methods of selecting subjects suitable for treatment with the combination therapies of the disclosure. As used herein, subjects who are considered suitable for treatment are those subjects who are expected to benefit from, or respond to, the combination treatment.

To “benefit from” or “respond to” can refer to any beneficial therapeutic effect observed in the subject to which a treatment was administered. This may be any overall clinical benefit derived from the treatment—for example, prolonged survival, partial or complete disease remission, slowing or absence of disease progression, tumour shrinkage (for example, a reduction in tumour volume of 5, 10, 20, 30, 40% or more), reduction in tumour burden (for example, a reduction in tumour burden of 5, 10, 20, 30, 40% or more), slowing or absence of tumour enlargement, slowing or absence of increase in tumour burden, or improved quality of life. To “not benefit from” or “not respond to” refers to the absence of these measures of overall clinical benefit derived from a treatment. “Additional benefit” can refer to the extra overall clinical benefit (for example, as assessed by any of the outcomes described above) derived from additional therapeutic agents when administered in combination (and which may be cumulative or synergistic).

In some cases, subjects may be selected on the basis of the amount or pattern of expression or activity of a marker protein of interest, for example STK11, STK11IP, KRAS, or p53. In some cases, subjects may be selected on the basis of a reduced presence of CD8⁺ cells having TCF1 activity or expression. In some cases, subjects may be selected based on response/benefit (or lack thereof) derived from a previously administered treatment, for example an immune checkpoint modulator, or a combination treatment comprising a chemotherapeutic agent and an immune checkpoint modulator. In some cases subjects may be selected on the basis of both: the amount or pattern of expression or activity of a marker protein of interest; and, response/benefit (or lack thereof) derived from a previously administered treatment.

Thus, in one aspect, the present disclosure provides a method of selecting a subject for treatment with a combination of an AXL inhibitor (AXLi) and an immune checkpoint modulator (ICM), the method comprising: identifying subjects having an AXL-related disease characterised by the presence of cells having modified STK11 activity or expression; and, selecting thus identified subjects for treatment. In some embodiments, the modified STK11 activity or expression is decreased STK11 activity or expression. In some embodiments, the method may further comprise: identifying subjects that have previously been treated with an immune checkpoint modulator (ICM) and which did not respond to treatment with the ICM; and, selecting thus identified subjects for treatment.

In another aspect, the present disclosure provides a method of selecting a subject for treatment with a combination of an AXL inhibitor (AXLi) and an immune checkpoint modulator (ICM), the method comprising: identifying subjects that have previously been treated with an immune checkpoint modulator (ICM) and which did not respond to treatment with the ICM; and, selecting thus identified subjects for treatment. In some embodiments, the method may further comprise: identifying subjects having an AXL-related disease characterised by the presence of cells having modified STK11 activity or expression; and, selecting thus identified subjects for treatment. In some embodiments, the modified STK11 activity or expression is decreased STK11 activity or expression.

In a further aspect, the present disclosure provides a method of selecting a subject for treatment with a combination of: an AXL inhibitor (AXLi); an immune checkpoint modulator (ICM); and, a chemotherapeutic agent and/or radiotherapy. The method may comprise: identifying subjects having an AXL-related disease characterised by the presence of cells having modified STK11 activity or expression; and, selecting thus identified subjects for treatment. In some embodiments, the modified STK11 activity or expression is decreased STK11 activity or expression In some embodiments, the method may further comprise: identifying subjects that have previously been treated with a combination of: an immune checkpoint modulator (ICM); and, a chemotherapeutic agent and/or radiotherapy; wherein treatment with the combination of immune checkpoint modulator (ICM) and chemotherapeutic agent and/or radiotherapy did not provide any additional benefit as compared to treatment with the chemotherapeutic agent and/or radiotherapy alone; and, selecting thus identified subjects for treatment.

In another aspect, the present disclosure provides a method of selecting a subject for treatment with a combination of: an AXL inhibitor (AXLi); an immune checkpoint modulator (ICM); and, a chemotherapeutic agent and/or radiotherapy; the method comprising: identifying subjects that have previously been treated with a combination of: an immune checkpoint modulator (ICM); and a chemotherapeutic agent and/or radiotherapy; wherein treatment with the combination of immune checkpoint modulator (ICM) and chemotherapeutic agent and/or radiotherapy did not provide any additional benefit as compared to treatment with the chemotherapeutic agent and/or radiotherapy alone; and, selecting thus identified subjects for treatment. In some embodiments, the method may further comprise: identifying subjects having an AXL-related disease characterised by the presence of cells having modified STK11 activity or expression; and, selecting thus identified subjects for treatment. In some embodiments, the modified STK11 activity or expression is decreased STK11 activity or expression

Embodiments of these aspects of the disclosure include:

A method of selecting a subject for treatment with a combination of: an AXL inhibitor (AXLi); an immune checkpoint modulator (ICM); and, a chemotherapeutic agent; the method comprising: identifying subjects having an AXL-related disease characterised by the presence of cells having modified STK11 activity or expression; and, selecting thus identified subjects for treatment. In some embodiments, the modified STK11 activity or expression is decreased STK11 activity or expression

A method of selecting a subject for treatment with a combination of: an AXL inhibitor (AXLi); an immune checkpoint modulator (ICM); and, radiotherapy; the method comprising: identifying subjects having an AXL-related disease characterised by the presence of cells having modified STK11 activity or expression; and, selecting thus identified subjects for treatment. In some embodiments, the modified STK11 activity or expression is decreased STK11 activity or expression.

A method of selecting a subject for treatment with a combination of: an AXL inhibitor (AXLi); an immune checkpoint modulator (ICM); a chemotherapeutic agent; and, radiotherapy; the method comprising: identifying subjects having an AXL-related disease characterised by the presence of cells having modified STK11 activity or expression; and, selecting thus identified subjects for treatment. In some embodiments, the modified STK11 activity or expression is decreased STK11 activity or expression.

A method of selecting a subject for treatment with a combination of: an AXL inhibitor (AXLi); an immune checkpoint modulator (ICM); and, a chemotherapeutic agent; the method comprising: identifying subjects that have previously been treated with a combination of: an immune checkpoint modulator (ICM); and a chemotherapeutic agent; wherein treatment with the combination of immune checkpoint modulator (ICM) and chemotherapeutic agent did not provide any additional benefit as compared to treatment with the chemotherapeutic agent alone; and, selecting thus identified subjects for treatment.

A method of selecting a subject for treatment with a combination of: an AXL inhibitor (AXLi); an immune checkpoint modulator (ICM); and, radiotherapy; the method comprising: identifying subjects that have previously been treated with a combination of: an immune checkpoint modulator (ICM); and radiotherapy; wherein treatment with the combination of immune checkpoint modulator (ICM) and radiotherapy did not provide any additional benefit as compared to treatment with radiotherapy alone; and, selecting thus identified subjects for treatment.

A method of selecting a subject for treatment with a combination of: an AXL inhibitor (AXLi); an immune checkpoint modulator (ICM); a chemotherapeutic agent; and, radiotherapy; the method comprising: identifying subjects that have previously been treated with a combination of: an immune checkpoint modulator (ICM); a chemotherapeutic agent; and, radiotherapy; wherein treatment with the combination of immune checkpoint modulator (ICM), chemotherapeutic agent, and radiotherapy did not provide any additional benefit as compared to treatment with the combination of chemotherapeutic agent and radiotherapy without ICM; and, selecting thus identified subjects for treatment.

In some embodiments of the methods of the disclosure outlined above, the method may comprise: identifying subjects wherein the AXL-related disease is further characterised by the presence of cells having increased KRAS activity or expression; and, selecting thus identified subjects for treatment.

In some embodiments of the methods of the disclosure, the method may comprise: identifying subjects wherein the AXL-related disease is further characterised by the presence of cells having decreased p53 activity or expression; and, selecting thus identified subjects for treatment.

In some embodiments of the methods of the disclosure, the method may comprise: identifying subjects wherein the AXL-related disease is further characterised by the presence of cells having increased AXL activity or expression; and, selecting thus identified subjects for treatment.

In some embodiments of the method of the disclosure, the method may comprise: identifying subjects having an AXL-related disease characterised by a reduced presence of CD8⁺ cells having TCF1 activity or expression; and, selecting thus identified subjects for treatment.

A subject who “did not respond” to treatment with an ICM is a subject who did not derive any clinical benefit from treatment with the ICM (which may have been administered alone or as one component of a treatment regime, for example a treatment regime that does not include an AXLi). In some embodiments, this may be a subject in which no slowing or absence of disease progression was observed following treatment with the ICM, or no slowing or absence of disease progression was attributable to the ICM. In some embodiments, this may be a subject in which no reduction in tumour volume was observed following treatment with the ICM, or no reduction in tumour volume was attributable to the ICM. In some embodiments, this may be a subject in which a less than 1, 2, 3, 4, 5, 6, 7, 8, 9, or 10% reduction in tumor volume was observed following treatment with the ICM, or less than 1, 2, 3, 4, 5, 6, 7, 8, 9, or 10% reduction in tumor volume was attributable to the ICM. In some embodiments, this may be a subject in which no reduction in tumour burden was observed following treatment with the ICM, or in which no reduction in tumour burden was attributable to the ICM. In some embodiments, this may be a subject in which a less than 1, 2, 3, 4, 5, 6, 7, 8, 9, or 10% reduction in tumor burden was observed following treatment with the ICM, or less than 1, 2, 3, 4, 5, 6, 7, 8, 9, or 10% reduction in tumor burden was attributable to the ICM.

In some embodiments of the disclosure, a subject in which treatment with a combination of ICM and chemotherapeutic agent and/or radiotherapy “did not provide any additional benefit” as compared to treatment without ICM is a subject in which addition of the ICM to the treatment regime does not provide any additional clinical benefit (whether cumulative or synergistic) to any clinical benefit observed with the treatment regime without ICM. In some embodiments, this may be a subject in which no additional slowing or absence of disease progression was observed following treatment with the combination treatment including ICM. In some embodiments, this may be a subject in which no additional reduction in tumour volume was observed following treatment with the combination treatment including ICM. In some embodiments, this may be a subject in which a less than 1, 2, 3, 4, 5, 6, 7, 8, 9, or 10% additional reduction in tumor volume was observed following treatment with the combination treatment including ICM. In some embodiments, this may be a subject in which no additional reduction in tumour burden was observed following treatment with the combination treatment including ICM. In some embodiments, this may be a subject in which a less than 1, 2, 3, 4, 5, 6, 7, 8, 9, or 10% additional reduction in tumor burden was observed following treatment with the combination treatment including ICM.

Assessing Increased/Decreased Expression

The term “expression” as used herein refers to the transcription of a gene's DNA template to produce the corresponding mRNA and translation of this mRNA to produce the corresponding gene product (i.e., a peptide, polypeptide, or protein) as well as the “expression” of a protein in one or more forms that may have been modified post translation.

Suitable means for determining or detecting level and patterns of expression, including gene expression, are readily known to those skilled in the art—for example, by microarray analysis, Western blotting or by PCR techniques such as QPCR. Altered expression may also be detected by analysing protein content of samples using methods such as ELISA, PET or SELDI-TOF MS, or by analytical techniques such as 2Dgel electrophoresis. Techniques such as this can be particularly useful for detecting altered expression in the form of alternative post translationally modified forms of a protein. In some embodiments, modified STK11 activity or expression may be assessed using the experimental methods described in the Examples (whole exome sequence analysis of tumour biopsy material).

In some embodiments of the disclosure, decreased STK11 activity or expression may be assessed by determining the level of activity or expression of STK11IP. In some embodiments, increased activity or expression of STK11IP relative to a control is indicative of decreased STK11 activity or expression. In some embodiments, decreased activity or expression of STK11IP relative to a control may be indicative of decreased STK11 activity or expression.

In some embodiments of the disclosure, increased STK11 activity or expression may be assessed by determining the level of activity or expression of STK11IP. In some embodiments, increased activity or expression of STK11IP relative to a control is indicative of increased STK11 activity or expression. In some embodiments, decreased activity or expression of STK11IP relative to a control may be indicative of increased STK11 activity or expression.

In some embodiments of the disclosure, modifed (increased or decreased) expression may be assessed by determining copy number of the gene encoding a protein of interest—for example, STK11, STK11IP, KRAS, or p53—relative to a control sample, wherein an increase in the copy number indicates an increased level of expression and a decrease in the copy number indicates a decreased level of expression. In some embodiments, modified (increased or decreased) expression is assessed by determining the level of a protein or mRNA of interest—for example, STK11, STK11IP, KRAS, or p53 protein or mRNA—relative to a control sample.

In some embodiments, modified (increased or decreased) activity or expression may be assessed by determining the presence or absence of a mutation in the nucleotide, mRNA, or amino acid protein sequence of a protein of interest. Suitable means for determining or detecting the presence or absence of such mutations are well known to those skilled in the art—for example, nucleotide sequencing, DNA hybridization, restriction enzyme digestion methods.

Thus, in some embodiments of the methods of the disclosure, modified STK11 activity or expression may be assessed by determining the presence or absence of a STK11 mutation and/or a STK11IP mutation. In some embodiments, increased KRAS activity or expression may be assessed by determining the presence or absence of a KRAS mutation. In some embodiments, decreased p53 activity or expression may be assessed by determining the presence or absence of a p53 mutation.

In some embodiments of the methods of the disclosure, the STK11 mutation, STK11IP mutation, KRAS mutation, and/or p53 mutation may be a mutation selected from: a mutation in the nucleotide sequence encoding STK11, STK11IP, KRAS, or p53; a mutation in a regulatory sequence controlling expression of the nucleotide sequence encoding STK11, STK11IP, KRAS, or p53; or, a mutation in a nucleotide encoding a protein which interacts with the transcription product of the STK11, STK11IP, KRAS, or p53 gene.

In some embodiments, the STK11 mutation, STK11IP mutation, KRAS mutation, and/or p53 mutation is a mutation in the translation product of the STK11, STK11IP, KRAS, or p53 gene. In some embodiments, the STK11 mutation, STK11IP mutation, KRAS mutation, and/or p53 mutation is a mutation in the transcription product of the STK11, STK11IP, KRAS, or p53 gene. In some embodiments, the STK11 mutation, STK11IP mutation, KRAS mutation, and/or p53 mutation is a mutation in a miRNA that regulates expression of STK11, STK11IP, KRAS, and/or p53.

In some embodiments of the disclosure, the STK11 mutation may be an inactivating mutation. In some embodiments of the disclosure, the STK11 mutation may be an activating mutation. In some embodiments of the disclosure the STK11IP mutation may be an activating mutation. In some embodiments of the disclosure the STK11IP mutation may be an inactivating mutation. In some embodiments, the KRAS mutation may be an activating mutation. In some preferred embodiments, the KRAS mutation may be a mutation at position G12, preferably a G12D mutation. In some embodiments, the p53 mutation may be an inactivating mutation. In some embodiments the STK11 mutation may be L160P, LD140PY, or D115V. In some embodiments of the disclosure, the STK11P mutation may be E30V, LG334FW, W162C, or R1065Q.

In some embodiments, the STK11 mutation may result in a reduced level of activity or expression of STK11 protein. In some embodiments, the STK11 mutation may result in an increased level of activity or expression of STK11 protein. In some embodiments, the STK11IP mutation may result in an increased level of activity or expression of STK11IP protein. In some embodiments, the STK11IP mutation may result in a decreased level of activity or expression of STK11IP protein. In some embodiments the STK11IP mutation may result in an altered pattern of activity or expression of STK11 protein, and/or altered subcellular localisation of STK11 protein. For example, increased cytosolic sequestration of STK11 protein and/or reduced localisation of STK11 protein to the nucleus. In some embodiments, the KRAS mutation may result in an increased level of activity or expression of KRAS protein. In some embodiments, the p53 mutation may result in a reduced level of activity or expression of p53 protein.

As used herein, an “activating” mutation is one which results in the transcription and/or translation product of a gene having increased function (which may be either increased activity or expression). As used herein, an “inactivating” mutation is one which results in the transcription and/or translation product of a gene having reduced or no function (which may be either decreased activity or expression). The mutation may be mutation in a nucleotide, mRNA, or protein sequence.

In some embodiments of the methods of the disclosure, the increased or decreased activity or expression is determined in a sample derived from a subject. That is, the methods of the disclosure may be performed in vitro or ex vivo on a sample isolated from a subject. The sample may comprise or may be derived from: a quantity of blood; a quantity of serum derived from the subject's blood which may comprise the fluid portion of the blood obtained after removal of the fibrin clot and blood cells; a quantity of pancreatic juice; a tissue sample or biopsy; or cells isolated from said subject. A sample may be taken from any tissue or bodily fluid. In some embodiments, the sample may include or may be derived from a tissue sample, biopsy, resection or isolated cells from a subject. In some preferred embodiments, the sample may be a tissue sample. The sample may be a sample of tumor tissue, such as neoplastic tumor tissue. The sample may be one that is obtained by a tumor biopsy.

In some embodiments, the sample may be taken from a bodily fluid, more preferably one that circulates through the body. Accordingly, the sample may be a blood sample or lymph sample. In some embodiments, the sample is a urine sample or a saliva sample. In some other embodiments, the sample is a blood sample or blood-derived sample. The blood derived sample may be a selected fraction of a subject's blood, e.g. a selected cell-containing fraction or a plasma or serum fraction.

A selected cell-containing fraction may contain cell types of interest which may include white blood cells (WBC), particularly peripheral blood mononuclear cells (PBC) and/or granulocytes, and/or red blood cells (RBC). Accordingly, methods according to the present disclosure may involve detection of a marker polypeptide or nucleic acid in the blood, in white blood cells, peripheral blood mononuclear cells, granulocytes and/or red blood cells.

The sample may be fresh or archival. For example, archival tissue may be from the first diagnosis of a subject, or a biopsy at a relapse. In some preferred embodiments, the sample may be a fresh biopsy.

Increased or decreased activity or expression of a marker protein of interest may be determined relative to a control. In some embodiments, target expression or activity in the subject is compared to target expression or activity in a control. In some cases, the control may be a reference sample or a reference dataset. The reference may be a sample that has been previously obtained from an individual or individuals with a known degree of suitability—for example, from an individual or individuals known to be responsive to the combination therapies disclosed herein. The reference may be a dataset obtained from analyzing a reference sample.

Controls may be positive controls in which the target molecule is known to be present, or expressed at high level, or may be negative controls in which the target molecule is known to be absent or expressed at low level. Controls may be samples of tissue that are from individuals who are known to benefit from the treatment. The tissue may be of the same type as the sample being tested. For example, a sample of tumor tissue from an individual may be compared to a control sample of tumor tissue from an individual who is known to be suitable for the treatment, such as an individual who has previously responded to the treatment. In some cases the control may be a sample obtained from the same individual as the test sample, but from a tissue known to be healthy. Thus, a sample of cancerous tissue from an individual may be compared to a non-cancerous tissue sample. In some cases, the control is a cell culture sample.

In some cases, the control may be a sample from a comparable AXL-related disease that is not characterized by modified activity or expression of STK11, STK11IP, KRAS, and/or p53. In some other cases, the control may be a sample of healthy tissue. In some preferred embodiments, the control is of the same sample type as the test sample—for example, a sample of the same tissue type as the AXL-related disease. In some other preferred embodiments, the control is a reference sample or dataset obtained from an individual or individuals known to be responsive to the combination therapies disclosed herein.

Assessing Increased/Decreased Presence of CD8+ Cells Having TCF1 Activity or Expression

The term “presence” as used herein refers to the population of CD8⁺ cells in a subject.

Suitable means for determining or detecting the presence of CD8⁺ cells are readily known to those skilled in the art. For example, flow cytometry, immunohistochemistry, single cell RNA sequencing and/or in situ PCR. In some embodiments, a reduced presence of CD8⁺ cells having TCF1 activity or expression may be assessed using the experimental methods described in the Examples.

In some embodiments of the methods of the disclosure, reduced presence of CD8⁺ cells having TCF1 activity or expression is determined in a sample derived from a subject. That is, the methods of the disclosure can be performed in vitro or ex vivo on a sample isolated from a subject. A sample can comprise or can be derived from: a quantity of blood; a quantity of serum derived from the subject's blood which can comprise the fluid portion of the blood obtained after removal of the fibrin clot and blood cells; a quantity of pancreatic juice; a tissue sample or biopsy; or cells isolated from a subject. A sample can be taken from any tissue or bodily fluid. In some embodiments, a sample can include or can be derived from a tissue sample, biopsy, resection or isolated cells from a subject. In some preferred embodiments, a sample can be a tissue sample. A sample can be a sample of tumor tissue, such as neoplastic tumor tissue. A sample can be one that is obtained by a tumor biopsy.

In some embodiments, a sample can be taken from a bodily fluid, more preferably one that circulates through the body. Accordingly, the sample can be a blood sample or lymph sample. In some embodiments, a sample is a urine sample or a saliva sample. In some other embodiments, a sample is a blood sample or blood-derived sample. A blood derived sample can be a selected fraction of a subject's blood, e.g. a selected cell-containing fraction or a plasma or serum fraction.

A selected cell-containing fraction can contain cell types of interest which may include white blood cells (WBC), particularly peripheral blood mononuclear cells (PBC) and/or granulocytes, and/or red blood cells (RBC). Accordingly, methods according to the present disclosure can involve detection of a marker polypeptide or nucleic acid in the blood, in white blood cells, peripheral blood mononuclear cells, granulocytes and/or red blood cells.

A sample may be fresh or archival. For example, archival tissue can be from the first diagnosis of a subject, or a biopsy at a relapse. In some preferred embodiments, a sample can be a fresh biopsy.

Increased or decreased activity or expression of a marker protein of interest can be determined relative to a control. In some embodiments, target expression or activity in the subject is compared to target expression or activity in a control. In some cases, a control can be a reference sample or a reference dataset. A reference can be a sample that has been previously obtained from an individual or individuals with a known degree of suitability—for example, from an individual or individuals known to be responsive to the combination therapies disclosed herein. A reference can be a dataset obtained from analyzing a reference sample.

Controls can be positive controls in which the target molecule is known to be present, or expressed at high level, or can be negative controls in which a target molecule is known to be absent or expressed at low level. Controls can be samples of tissue that are from individuals who are known to benefit from the treatment. The tissue can be of the same type as the sample being tested. For example, a sample of tumor tissue from an individual can be compared to a control sample of tumor tissue from an individual who is known to be suitable for the treatment, such as an individual who has previously responded to the treatment. In some cases the control can be a sample obtained from the same individual as the test sample, but from a tissue known to be healthy. Thus, a sample of cancerous tissue from an individual can be compared to a non-cancerous tissue sample. In some cases, the control is a cell culture sample.

In some cases, the control can be a sample from a comparable AXL-related disease that is not characterized by reduced presence of CD8+ cells having TCF1 activity or expression. In some other cases, the control can be a sample of healthy tissue. In some preferred embodiments, the control is of the same sample type as the test sample—for example, a sample of the same tissue type as the AXL-related disease. In some other preferred embodiments, the control is a reference sample or dataset obtained from an individual or individuals known to be responsive to the combination therapies disclosed herein.

Assessing the Population of Desired T Cells in a Subject

Suitable means for determining a population of desired T cells (for example, TCF1⁺ T cells, PD-1⁺ T cells, CD8⁺ T cells, TCF1⁺ PD-1⁺ T cells, or TCF1⁺ PD-1⁺ CD8⁺ T cells, or T cells having any other combination of these markers) in a subject will be known to the person skilled in the art. For example, flow cytometry, immunohistochemistry, single cell RNA sequencing and/or in situ PCR.

In some embodiments of the methods of the disclosure, a population of desired T cells is determined in a sample derived from a subject. That is, the methods of the disclosure can be performed in vitro or ex vivo on a sample isolated from a subject. A sample can comprise or can be derived from: a quantity of blood; a quantity of serum derived from the subject's blood which can comprise the fluid portion of the blood obtained after removal of the fibrin clot and blood cells; a quantity of pancreatic juice; a tissue sample or biopsy; or cells isolated from said subject. A sample can be taken from any tissue or bodily fluid. In some embodiments, a sample can include or can be derived from a tissue sample, biopsy, resection or isolated cells from a subject. In some preferred embodiments, a sample can be a tissue sample. A sample can be a sample of tumor tissue, such as neoplastic tumor tissue. A sample can be one that is obtained by a tumor biopsy.

In some embodiments, a sample can be taken from a bodily fluid, more preferably one that circulates through the body. Accordingly, a sample can be a blood sample or lymph sample. In some embodiments, a sample is a urine sample or a saliva sample. In some other embodiments, a sample is a blood sample or blood-derived sample. The blood derived sample can be a selected fraction of a subject's blood, e.g. a selected cell-containing fraction or a plasma or serum fraction.

A selected cell-containing fraction can contain cell types of interest which can include white blood cells (WBC), particularly peripheral blood mononuclear cells (PBC) and/or granulocytes, and/or red blood cells (RBC). Accordingly, methods according to the present disclosure can involve detection of a marker polypeptide or nucleic acid in the blood, in white blood cells, peripheral blood mononuclear cells, granulocytes and/or red blood cells.

A sample can be fresh or archival. For example, archival tissue can be from the first diagnosis of a subject, or a biopsy at a relapse. In some preferred embodiments, a sample can be a fresh biopsy.

Increased or decreased activity or expression of a marker protein of interest can be determined relative to a control. In some embodiments, target expression or activity in the subject is compared to target expression or activity in a control. In some cases, a control can be a reference sample or a reference dataset. A reference can be a sample that has been previously obtained from an individual or individuals with a known degree of suitability—for example, from an individual or individuals known to be responsive to the combination therapies disclosed herein. A reference can be a dataset obtained from analyzing a reference sample.

Controls can be positive controls in which the target molecule is known to be present, or expressed at high level, or can be negative controls in which the target molecule is known to be absent or expressed at low level. Controls can be samples of tissue that are from individuals who are known to benefit from the treatment. The tissue can be of the same type as the sample being tested. For example, a sample of tumor tissue from an individual can be compared to a control sample of tumor tissue from an individual who is known to be suitable for the treatment, such as an individual who has previously responded to the treatment. In some cases the control can be a sample obtained from the same individual as the test sample, but from a tissue known to be healthy. Thus, a sample of cancerous tissue from an individual can be compared to a non-cancerous tissue sample. In some cases, a control is a cell culture sample.

AXL-Related Disease

As referred to herein, an AXL-related disease is one which in which dysfunction of Axl expression or activity is a contributing factor. For example, the AXL-related disease may be one in which overexpression of AXL is a contributing factor. Overexpression of AXL and/or its ligand has been reported in a wide variety of solid tumor types, as well as in other disease states including vascular injury and kidney disease [citations, incorporated by reference].

In some embodiments of the disclosure the AXL-related disease is a proliferative disease. A proliferative disease in one in which excessive proliferation of cells contributes to the pathogenesis of the disease. Exemplary proliferative diseases include: cancer, atherosclerosis, rheumatoid arthritis, psoriasis, idiopathic pulmonary fibrosis, scleroderma, and cirrhosis of the liver.

In some embodiments of the disclosure the AXL-related disease is a neoplastic disease. A neoplastic disease in one in which abnormal and excessive growth (termed neoplasia) of cells/tissue occurs. Neoplasia is the abnormal growth and proliferation of abnormal cells or abnormal amounts of cells, which can be due to a benign or malignant process. Exemplary neoplastic diseases include: myeloproliferative diseases, myelodysplastic syndromes (MDS), and acute myeloid leukemias (AML).

In some preferred embodiments of the disclosure, the AXL-related disease is cancer.

In some embodiments, the cancer may be one or more of the following cancers: Leukemias such as but not limited to acute myelocytic leukemias (AMLs) such as myeloblastic, promyelocytic, myelomonocytic, monocytic, erythroleukemia leukemias and myelodysplastic syndrome, acute leukemia, acute lymphocytic leukemia, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, metastatic cancers, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer, including, but not limited to, adenocarcinoma, lobular (small cell) carcinoma, intraductal carcinoma, medullary breast cancer, mucinous breast cancer, tubular breast cancer, papillary breast cancer, primary cancers, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; genital cancers such as penile cancer; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas. Preferably, the cancer may be selected from lung, breast, melanoma, prostate, ovarian, colorectal, or glioma cancer. The cancer may be metastatic. Most preferably the cancer is lung cancer.

In some embodiments, the cancer may be one or more solid cancer tumors, including, but not limited to, lung cancer, non-small cell lung carcinoma, breast, renal, endometrial, ovarian, thyroid, and, melanoma, prostate carcinoma, sarcoma, gastric cancer and uveal melanoma; liquid tumors, including but not limited to, leukemias (particularly myeloid leukemias) and lymphomas.

In some embodiments, the cancer may be one or more leukaemias such as but not limited to, acute leukemia, acute lymphocytic leukemia, acute myeloid leukemia, acute myelocytic leukaemias such as myeloblastic, promyelocytic, myelomonocytic, monocytic, erythroleukaemia leukaemias and myelodysplastic syndrome, chronic leukaemias such as but not limited to, chronic myelocytic (granulocytic) leukemia, chronic lymphocytic leukemia, hairy cell leukemia; polycythemia vera;

In some embodiments, the cancer may be one or more lymphomas such as but not limited to Hodgkin's disease, non-Hodgkin's disease.

In some preferred embodiments, the AXL-related disease may be a solid tumour. In other preferred embodiments, the AXL-related disease may be a cancer selected from the group consisting of: lung cancer, small-cell lung cancer, non-small-cell lung cancer, histocytoma, glioma, astrocyoma, osteoma, gastrointestinal cancer, bowel cancer, colon cancer, breast cancer, ovarian carcinoma, prostate cancer, testicular cancer, liver cancer, kidney cancer, urothelial carcinoma, bladder cancer, pancreas cancer, brain cancer, glioblastoma, sarcoma, osteosarcoma, Kaposi's sarcoma, melanoma, mesothelioma, lymphomas, and leukemias.

In some preferred embodiments, the AXL-related disease may be a cancer selected from the group consisting of: lung cancer, non-small-cell lung cancer, breast cancer, melanoma, mesothelioma, acute myeloid leukemia (AML), myelodysplatic syndrome (MDS), pancreas cancer, kidney cancer, urothelial carcinoma, and glioblastoma.

In some particularly preferred embodiments, the AXL-related disease may be a lung cancer. In some most preferred embodiments, the AXL-related disease may be non-small-cell lung cancer, such as lung adenocarcinoma.

In some embodiments of the disclosure, the AXL-related disease may be selected from: endometriosis, vascular disease/injury (including but not limited to restenosis, atherosclerosis and thrombosis), psoriasis; visual impairment due to macular degeneration; diabetic retinopathy and retinopathy of prematurity; kidney disease (including but not limited to glomerulonephritis, diabetic nephropathy and renal transplant rejection), rheumatoid arthritis; osteoarthritis, osteoporosis and cataracts.

In some embodiments of the disclosure, the AXL-related disease may be selected from: Immune disorders, cardiovascular disorders, thrombosis, diabetes, immune checkpoint disorders, fibrotic disorders (fibrosis), or proliferative diseases such as cancer, particularly metastatic cancer. Furthermore, Axl is known to play a role in many cancers of epithelial origin.

In some embodiments of the disclosure, the AXL-related disease may be fibrosis (including but not limited to lung fibrosis and liver fibrosis) or a fibrotic disorder. Fibrotic disorders of interest include strabismus, scleroderma, keloid, Nephrogenic systemic fibrosis, pulmonary fibrosis, idiopathic pulmonary fibrosis (IPF), cystic fibrosis (CF), systemic sclerosis, cardiac fibrosis, non-alcoholic steatohepatitis (NASH), and other types of liver fibrosis, primary biliary cirrhosis, renal fibrosis, cancer, and atherosclerosis. In these diseases, the chronic development of fibrosis in tissue leads to marked alterations in the architecture of the affected organs and subsequently cause defective organ function.

In some embodiments of the disclosure, the AXL-related disease may be an immune checkpoint disorder. Immune checkpoint disorders of interest include: Chronic viral infections, Melanoma, Colorectal cancer, Breast cancer, Ovarian cancer, Non-small cell lung cancer (NSCLC), Prostate cancer, Renal cell cancer, Pancreatic cancer, Esophagus cancer, Bladder cancer, Myeloma, Kidney cancer, Bladder cancer, Brain tumor, and Lymphoma.

In some embodiments, the AXL-related disease may not respond to or benefit from treatment with an immune checkpoint modulator (ICM) when administered alone or as part of a treatment regime that does not include an AXLi. To “not respond to”/“not benefit from” refers to the absence of a beneficial therapeutic effect observed in the subject to which a treatment was administered. This may be as assessed by one or more of the measures of clinical benefit described more fully elsewhere herein. For example, in embodiments in which the AXL-related disease is cancer, to “not respond to”/“not benefit from” treatment may mean: no slowing or absence of cancer progression; no reduction in tumour volume; less than 1, 2, 3, 4, 5, 6, 7, 8, 9, or 10% reduction in tumor volume; no reduction in tumour burden; and/or less than 1, 2, 3, 4, 5, 6, 7, 8, 9, or 10% reduction in tumor burden; is observed following treatment with an ICM when administered alone or as part of a treatment regime that does not include an AXLi.

In some embodiments, the AXL-related disease may be characterised by the amount or pattern of expression or activity of a marker protein of interest, for example STK11, STK11IP, KRAS, or p53. In some embodiments, the AXL-related disease may be characterised by the presence of cells having a mutation in one or more genes encoding a marker protein of interest, for example a gene encoding STK11, STK11IP, KRAS, or p53.

Thus, in some embodiments the AXL-related disease may be characterised by the presence of cells having modifed STK11 activity or expression. In some embodiments, the modified STK11 activity or expression is a reduced STK11 activity or expression. In some embodiments, the AXL-related disease may be characterised by the presence of cells having a STK11 mutation and/or a STK11IP mutation. In some embodiments the AXL-related disease may be characterised by the presence of cells having increased KRAS activity or expression. In some embodiments, the AXL-related disease may be characterised by the presence of cells having a KRAS mutation. In some embodiments the AXL-related disease may be characterised by the presence of cells having decreased p53 activity or expression. In some embodiments, the AXL-related disease may be characterised by the presence of cells having a p53 mutation. In some embodiments the AXL-related disease may be characterised by the presence of cells having increased AXL activity or expression. In some embodiments, the AXL-related disease may be characterised by the presence of cells having an AXL mutation.

In some embodiments, the AXL-related disease may be characterised by the presence of cells having modified STK11 activity or expression, preferably decreased STK11 activity or expression, increased KRAS activity or expression, and/or decreased p53 activity or expression. In some embodiments, the AXL-related disease may be characterised by the presence of cells having modified STK11 activity or expression, preferably decreased STK11 activity or expression, increased KRAS activity or expression, and decreased p53 activity or expression. In some embodiments, the AXL-related disease may be characterised by the presence of cells having a STK11 mutation and/or a STK11IP mutation, a KRAS mutation, and/or a p53 mutation. In some embodiments, the AXL-related disease may be characterised by the presence of cells having a STK11 mutation, a KRAS mutation, and a p53 mutation. In other embodiments, the AXL-related disease may be characterised by the presence of cells having a STK11IP mutation, a KRAS mutation, and a p53 mutation. In some embodiments, the AXL-related disease may be characterised by the presence of cells having a STK11 mutation, a STK11IP mutation, a KRAS mutation, and a p53 mutation.

In some cases, the AXL-related disease may not be an AXL-related disease characterised by: increased KRAS activity or expression, and wild-type STK11 and/or p53 activity or expression. In some cases, the AXL-related disease may not be an AXL-related disease characterised by: the presence of cells having a KRAS mutation; and, the absence of cells having a STK11, STK11IP, and/or p53 mutation. In some cases, the AXL-related disease may not be an AXL-related disease characterised by the presence of cells having a KRAS G12C mutation. In some cases, the AXL-related disease may not be an AXL-related disease characterised by cells which do not have a STK11, STK11IP, and/or p53 mutation. In some cases, the AXL-related disease is not a Lewis Lung Carcinoma (LLC) or Lewis Lung model tumour.

In the above described embodiments, increased/decreased activity of STK11, STK11IP, KRAS, and/or p53 may be determined as described in detail elsewhere herein. Similarly, in the above described embodiments the STK11 mutation, STK11IP mutation, KRAS mutation, and/or p53 mutation may be as described in detail elsewhere herein.

Methods of Treatment

As outlined above, the present disclosure provides methods of treating subjects selected by the disclosed “methods of selecting a subject” with combination therapies which comprise an AXL-kinase inhibitor and an immune regulatory agent.

Thus, the methods of selecting a subject described above may further comprise a step of treating the subject with the combination therapies disclosed herein. That is, in some embodiments, the methods of selecting a subject described above may further comprise a step of administering to the subject a therapeutically effective amount of a combination therapy of the disclosure. Such combination therapies include: a combination of an AXL inhibitor (AXLi) and an immune checkpoint modulator (ICM); a combination of an AXL inhibitor (AXLi), an immune checkpoint modulator (ICM), and a chemotherapeutic agent; a combination of an AXL inhibitor (AXLi), an immune checkpoint modulator (ICM), and radiotherapy; and, a combination of an AXL inhibitor (AXLi), an immune checkpoint modulator (ICM), a chemotherapeutic agent, and radiotherapy.

The present disclosure thus also provides methods of treating an AXL-related disease in a subject with the combination therapies disclosed herein. The AXL-related disease may be as defined in detail elsewhere herein. Accordingly, the methods of treatment of the disclosure include:

A method of treating an AXL-related disease in a subject with a combination of an AXL inhibitor (AXLi) and an immune checkpoint modulator (ICM), the method comprising: (i) administering a combination of the AXLi and the ICM to the subject; (ii) administering the AXLi to the subject, wherein the ICM has been, is, or will be, administered to the subject; or (iii) administering the ICM to the subject, wherein the AXLi has been, is, or will be, administered to the subject; wherein the AXL-related disease is characterised by the presence of cells having modified STK11 activity or expression. In some embodiments, the modified STK11 activity or expression is a decreased STK11 activity or expression.

A method of treating an AXL-related disease in a subject with a combination of an AXL inhibitor (AXLi) and an immune checkpoint modulator (ICM), the method comprising: (i) administering a combination of the AXLi and the ICM to the subject; (ii) administering the AXLi to the subject, wherein the ICM has been, is, or will be, administered to the subject; or (iii) administering the ICM to the subject, wherein the AXLi has been, is, or will be, administered to the subject; wherein the AXL-related disease is characterised by the presence of cells having a STK11 mutation or a STK11I P mutation.

A method of treating an AXL-related disease in a subject with a combination of an AXL inhibitor (AXLi) and an immune checkpoint modulator (ICM), the method comprising: (i) administering a combination of the AXLi and the ICM to the subject; (ii) administering the AXLi to the subject, wherein the ICM has been, is, or will be, administered to the subject; or (iii) administering the ICM to the subject, wherein the AXLi has been, is, or will be, administered to the subject; wherein the subject has been selected for treatment on the basis that the AXL-related disease is characterised by the presence of cells having modified STK11 activity or expression, preferably decreased STK11 activity or expression.

A method of treating an AXL-related disease in a subject with a combination of an AXL inhibitor (AXLi) and an immune checkpoint modulator (ICM), the method comprising: (i) administering a combination of the AXLi and the ICM to the subject; (ii) administering the AXLi to the subject, wherein the ICM has been, is, or will be, administered to the subject; or (iii) administering the ICM to the subject, wherein the AXLi has been, is, or will be, administered to the subject; wherein the subject has been selected for treatment using a method of selecting a subject as described elsewhere herein.

A method of treating an AXL-related disease in a subject, the method comprising administering to a subject in need thereof a therapeutically effective amount of an AXL inhibitor, wherein the AXL inhibitor is administered in combination with: an immune checkpoint modulator (ICM); and, a chemotherapeutic agent and/or radiotherapy; wherein the AXL-related disease is characterised by the presence of cells having modified STK11 activity or expression. In some embodiments, the modified STK11 activity or expression is decreased STK11 activity or expression.

A method of treating an AXL-related disease in a subject, the method comprising administering to a subject in need thereof a therapeutically effective amount of an AXL inhibitor, wherein the AXL inhibitor is administered in combination with: an immune checkpoint modulator (ICM); and, a chemotherapeutic agent and/or radiotherapy; wherein the AXL-related disease is characterised by the presence of cells having a STK11 mutation or a STK11I P mutation.

A method of treating an AXL-related disease in a subject, the method comprising administering to a subject in need thereof a therapeutically effective amount of an AXL inhibitor, wherein the AXL inhibitor is administered in combination with: an immune checkpoint modulator (ICM); and, a chemotherapeutic agent and/or radiotherapy; wherein the subject has been selected for treatment on the basis that the AXL-related disease is characterised by the presence of cells having modified STK11 activity or expression. In some embodiments, the modified STK11 activity or expression is decreased STK11 activity or expression.

A method of treating an AXL-related disease in a subject, the method comprising administering to a subject in need thereof a therapeutically effective amount of an AXL inhibitor, wherein the AXL inhibitor is administered in combination with: an immune checkpoint modulator (ICM); and, a chemotherapeutic agent and/or radiotherapy; wherein the subject has been selected for treatment using a method of selecting a subject as described elsewhere herein.

The present authors believe that it may not be required to administer the AXL inhibitor, immune checkpoint modulator (ICM), and chemotherapeutic agent and/or radiotherapy to the subject simultaneously in order to achieve the enhanced efficacy associated with these combination therapies. Thus, as used herein, “administration in combination” may mean concurrent administration or may mean separate and/or sequential administration in any order.

Thus, in some embodiments, the AXL inhibitor may be administered concurrently with the immune checkpoint modulator (ICM). In some embodiments, the AXL inhibitor may be administered concurrently with the chemotherapeutic agent. In some embodiments, the AXL inhibitor may be administered concurrently with radiotherapy. In some embodiments, the AXL inhibitor may be administered concurrently with the ICM, chemotherapeutic agent, and/or radiotherapy.

In other embodiments, the AXL inhibitor may be administered separately and/or sequentially to the immune checkpoint modulator (ICM). In some embodiments, the AXL inhibitor may be administered separately and/or sequentially to the chemotherapeutic agent. In some embodiments, the AXL inhibitor may be administered separately and/or sequentially to radiotherapy. In some embodiments, the AXL inhibitor may be administered separately and/or sequentially to the ICM, chemotherapeutic agent, and/or radiotherapy.

In some embodiments, the AXL inhibitor may be administered prior to administration of the immune checkpoint modulator (ICM). In some embodiments, the ICM may be administered prior to administration of the AXL inhibitor. In some embodiments, the AXL inhibitor may be administered subsequent to administration of the immune checkpoint modulator (ICM). In some embodiments, the ICM may be administered subsequent to administration of the AXL inhibitor.

In some embodiments of the disclosure, the AXL inhibitor, immune checkpoint modulator (ICM), and chemotherapeutic agent may be administered concurrently. In other embodiments the AXL inhibitor, immune checkpoint modulator (ICM), and chemotherapeutic agent may be administered separately and/or sequentially. In some embodiments of the disclosure, the AXL inhibitor, immune checkpoint modulator (ICM), and radiotherapy may be administered concurrently. In other embodiments the AXL inhibitor, immune checkpoint modulator (ICM), and radiotherapy may be administered separately and/or sequentially. In some embodiments, the AXL inhibitor, immune checkpoint modulator (ICM), and chemotherapeutic agent and/or radiotherapy may be administered concurrently. In other embodiments the AXL inhibitor, immune checkpoint modulator (ICM), and chemotherapeutic agent and/or radiotherapy may be administered separately and/or sequentially.

In some embodiments, the AXL inhibitor may be administered concurrently with the immune checkpoint modulator (ICM) and/or the chemotherapeutic agent. In some embodiments, the AXL inhibitor may be administered concurrently with the immune checkpoint modulator (ICM) and/or the radiotherapy. In some embodiments, the AXL inhibitor may be administered concurrently with the immune checkpoint modulator (ICM) and/or the chemotherapeutic agent and radiotherapy. In some embodiments, the AXL inhibitor may be administered subsequent to administration of the immune checkpoint modulator (ICM) and/or subsequent to administration of the chemotherapeutic agent. In some embodiments, the AXL inhibitor may be administered subsequent to administration of the immune checkpoint modulator (ICM) and/or subsequent to administration of radiotherapy. In some embodiments, the AXL inhibitor may be administered subsequent to administration of the immune checkpoint modulator (ICM) and/or subsequent to administration of the chemotherapeutic agent and radiotherapy.

In some embodiments, the AXL inhibitor may be administered subsequent to administration of the immune checkpoint modulator (ICM) and the chemotherapeutic agent. In some embodiments, the AXL inhibitor may be administered subsequent to administration of the immune checkpoint modulator (ICM) and radiotherapy. In some embodiments, the AXL inhibitor may be administered subsequent to administration of the immune checkpoint modulator (ICM) and the chemotherapeutic agent and radiotherapy. In some other embodiments, the AXL inhibitor may be administered prior to administration of the immune checkpoint modulator (ICM) and/or prior to administration of the chemotherapeutic agent. In some embodiments, the AXL inhibitor may be administered prior to administration of the immune checkpoint modulator (ICM) and/or prior to administration of radiotherapy. In some other embodiments, the AXL inhibitor may be administered prior to administration of the immune checkpoint modulator (ICM) and/or prior to administration of the chemotherapeutic agent and radiotherapy.

In some embodiments, the AXL inhibitor may be administered prior to administration of the immune checkpoint modulator (ICM) and the chemotherapeutic agent. In some embodiments, the AXL inhibitor may be administered prior to administration of the immune checkpoint modulator (ICM) and radiotherapy. In some embodiments, the AXL inhibitor may be administered prior to administration of the immune checkpoint modulator (ICM) and the chemotherapeutic agent and radiotherapy.

In some embodiments, the AXL inhibitor may be administered subsequent to administration of the chemotherapeutic agent, and the immune checkpoint modulator (ICM) may be administered subsequent to administration of the AXL inhibitor. In some embodiments, the AXL inhibitor may be administered prior to administration of the chemotherapeutic agent, and the immune checkpoint modulator (ICM) may be administered prior to administration of the AXL inhibitor. In some embodiments, the AXL inhibitor may be administered subsequent to administration of radiotherapy, and the immune checkpoint modulator (ICM) may be administered subsequent to administration of the AXL inhibitor. In some embodiments, the AXL inhibitor may be administered prior to administration of radiotherapy, and the immune checkpoint modulator (ICM) may be administered prior to administration of the AXL inhibitor.

In some embodiments, the immune checkpoint modulator (ICM) may be administered subsequent to administration of the AXL inhibitor and/or subsequent to administration of the chemotherapeutic agent and/or radiotherapy. In some embodiments, the immune checkpoint modulator (ICM) may be administered subsequent to administration of the AXL inhibitor and the chemotherapeutic agent and/or radiotherapy. In some other embodiments, the immune checkpoint modulator (ICM) may be administered prior to administration of the AXL inhibitor and/or prior to administration of the chemotherapeutic agent and/or radiotherapy. In some embodiments, the immune checkpoint modulator (ICM) may be administered prior to administration of the AXL inhibitor and the chemotherapeutic agent and/or radiotherapy. In some embodiments, the immune checkpoint modulator (ICM) may be administered subsequent to administration of the AXL inhibitor, and the chemotherapeutic agent and/or radiotherapy may be administered subsequent to administration of the immune checkpoint modulator (ICM). In some embodiments, the immune checkpoint modulator (ICM) may be administered prior to administration of the AXL inhibitor, and the chemotherapeutic agent and/or radiotherapy may be administered prior to administration of the immune checkpoint modulator (ICM).

In some embodiments, the chemotherapeutic agent and/radiotherapy may be administered subsequent to administration of the AXL inhibitor and/or subsequent to administration of the immune checkpoint modulator (ICM). In some embodiments, the chemotherapeutic agent and/or radiotherapy may be administered subsequent to administration of the AXL inhibitor and the immune checkpoint modulator (ICM). In some other embodiments, the chemotherapeutic agent and/or radiotherapy may be administered prior to administration of the AXL inhibitor and/or prior to administration of the immune checkpoint modulator (ICM). In some embodiments, the chemotherapeutic agent and/or radiotherapy may be administered prior to administration of the AXL inhibitor and the immune checkpoint modulator (ICM). In some embodiments, the chemotherapeutic agent and/or radiotherapy may be administered subsequent to administration of the AXL inhibitor, and the immune checkpoint modulator (ICM) may be administered subsequent to administration of the chemotherapeutic agent and/or radiotherapy. In some other embodiments, the chemotherapeutic agent and/or radiotherapy may be administered prior to administration of the AXL inhibitor, and the immune checkpoint modulator (ICM) may be administered prior to administration of the chemotherapeutic agent and/or radiotherapy.

In some embodiments of the disclosed methods of treating an AXL-related disease, the method comprises: administering the AXL inhibitor to the subject, when the immune checkpoint modulator (ICM) has been, is, or will be, administered to the subject; and/or administering the AXL inhibitor to the subject, when the chemotherapeutic agent and/or radiotherapy has been, is, or will be, administered to the subject.

In some embodiments of the disclosed methods of treating an AXL-related disease, the method comprises: administering the immune checkpoint modulator (ICM) to the subject, when the AXL inhibitor has been, is, or will be, administered to the subject; and/or administering the immune checkpoint modulator (ICM) to the subject, when the chemotherapeutic agent and/or radiotherapy has been, is, or will be, administered to the subject.

In some embodiments of the disclosed methods of treating an AXL-related disease, the method comprises: administering the chemotherapeutic agent and/or radiotherapy to the subject, when the AXL inhibitor has been, is, or will be, administered to the subject; and/or administering the chemotherapeutic agent and/or radiotherapy to the subject, wherein the immune checkpoint modulator (ICM) has been, is, or will be, administered to the subject.

In embodiments where the AXL inhibitor, immune checkpoint modulator (ICM), and chemotherapeutic agent and/or radiotherapy are not administered concurrently, preferably the AXL inhibitor and ICM are administered to the subject no more than 3 weeks apart, such as no more than 1 week apart, no more than 48 hours apart, or no more than 24 hours apart. In such embodiments, preferably the AXL inhibitor and chemotherapeutic agent and/or radiotherapy are administered to the subject no more than 4 weeks apart, such as no more than 3 weeks apart, no more than 1 week apart, no more than 48 hours apart, or no more than 24 hours apart.

Similarly, in embodiments where only one agent is administered as part of the described method (for example, methods involving administering an AXL inhibitor to a subject wherein an ICM has been or will be administered to the subject, and/or a chemotherapeutic agent and/or radiotherapy has been or will be administered to the subject) the method typically involves administering the AXL inhibitor to the subject no more than 3 weeks before/after the ICM and/or chemotherapeutic agent and/or radiotherapy has been or will be administered—such as no more than 1 week before/after, no more than 48 hours before/after, or no more than 24 hours before/after.

In some embodiments of the disclosed methods of treating an AXL-related disease, the Axl inhibitor may be administered to the subject daily, or every 2, 3, 4, 5, 6, or 7 days. In some embodiments in which the Axl inhibitor is Bemcentinib, the Axl inhibitor is preferably administered to the subject daily.

In some embodiments of the disclosed methods of treating an AXL-related disease, the immune checkpoint modulator (ICM) may be administered to the subject every 1, 2, 3, 4, 5, 6, or 7 weeks. In some preferred embodiments the immune checkpoint modulator (ICM) may be administered to the subject every 3 or 4 weeks. In some embodiments in which the immune checkpoint modulator (ICM) is Pembrolizumab, the immune checkpoint modulator (ICM) is preferably administered to the subject every 3 weeks. In some embodiments in which the immune checkpoint modulator (ICM) is durvalumab, the immune checkpoint modulator (ICM) is preferably administered to the subject every 4 weeks. In some embodiments in which the immune checkpoint modulator (ICM) is durvalumab and tremelimumab, the immune checkpoint modulator (ICM) is preferably administered to the subject every 4 weeks. In some embodiments in which the immune checkpoint modulator (ICM) is ipililumab and nivolumab, the immune checkpoint modulator (ICM) is preferably administered to the subject every 2, 3, or 4 weeks.

In some embodiments of the disclosed methods of treating an AXL-related disease, the chemotherapeutic agent may be administered to the subject every 1, 2, 3, 4, 5, 6, or 7 weeks. In some preferred embodiments the chemotherapeutic agent may be administered to the subject every 3 or 4 weeks. In some embodiments in which the chemotherapeutic agent is doxorubicin, the chemotherapeutic agent is preferably administered to the subject every 3 weeks. In some embodiments in which the chemotherapeutic agent is doxorubicin in pegylated liposomal form, the chemotherapeutic agent is preferably administered to the subject every 4 weeks.

In some preferred embodiments of the disclosed methods of treating an AXL-related disease, the Axl inhibitor is administered to the subject daily, the immune checkpoint modulator (ICM) is administered to the subject every 4 weeks, and the chemotherapeutic agent is administered to the subject every 3 weeks.

Also provided are methods of treating an AXL-related disease, the method comprising administering to a subject in need thereof a therapeutically effective amount of an AXL inhibitor, wherein the subject has been or will be administered an immune checkpoint modulator and/or a chemotherapeutic agent and/or radiotherapy.

Also provided are methods of treating an AXL-related disease, the method comprising administering to a subject in need thereof a therapeutically effective amount of an immune checkpoint modulator (ICM), wherein the subject has been or will be administered an AXL inhibitor and/or a chemotherapeutic agent and/or radiotherapy.

Also provided are methods of treating an AXL-related disease, the method comprising administering to a subject in need thereof a therapeutically effective amount of a chemotherapeutic agent and/or radiotherapy, wherein the subject has been or will be administered an AXL inhibitor and/or an immune checkpoint modulator (ICM).

In some embodiments of the methods of the disclosure, the AXL inhibitor and ICM are administered to the subject no more than 4 weeks apart, such as no more than 3 weeks, no more than 1 week apart, no more than 48 hours apart, or no more than 24 hours apart. That is, in some embodiments the AXL inhibitor may be administered to the subject within 4 weeks, within 3 weeks, within 1 week, of the ICM being administered to the subject. For example, in some embodiments the AXL inhibitor may be administered to the subject 4 weeks, 3 weeks, or 1 week after administration of the ICM. In other embodiments, the AXL inhibitor may be administered to the subject 4 weeks, 3 weeks, or 1 week before administration of the ICM.

In some embodiments of the methods of the disclosure, the AXL inhibitor and chemotherapeutic agent are administered to the subject no more than 4 weeks apart, such as no more than 3 weeks, no more than 1 week apart, no more than 48 hours apart, or no more than 24 hours apart. That is, in some embodiments the AXL inhibitor may be administered to the subject within 4 weeks, within 3 weeks, within 1 week, of the chemotherapeutic agent being administered to the subject. For example, in some embodiments the AXL inhibitor may be administered to the subject 4 weeks, 3 weeks, or 1 week after administration of the chemotherapeutic agent. In other embodiments, the AXL inhibitor may be administered to the subject 4 weeks, 3 weeks, or 1 week before administration of the chemotherapeutic agent.

In some embodiments of the methods of the disclosure, the ICM and chemotherapeutic agent are administered to the subject no more than 4 weeks apart, such as no more than 3 weeks, no more than 1 week apart, no more than 48 hours apart, or no more than 24 hours apart. That is, in some embodiments the ICM may be administered to the subject within 4 weeks, within 3 weeks, within 1 week, of the chemotherapeutic agent being administered to the subject. For example, in some embodiments the ICM may be administered to the subject 4 weeks, 3 weeks, or 1 week after administration of the chemotherapeutic agent. In other embodiments, the ICM may be administered to the subject 4 weeks, 3 weeks, or 1 week before administration of the chemotherapeutic agent.

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

Typically, in the methods of treatment described herein the agents (AXL inhibitors, ICMs, chemotherapeutic agents) are administered in a therapeutically or prophylactically effective amount. The term “therapeutically-effective amount” or “effective amount” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

Typically, the subjects treated are in need of the described treatment.

A “therapeutically effective amount” is an amount sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

The disclosed methods of treatment may involve administration of the above described combination therapies either alone or in further combination with other treatments, either simultaneously or sequentially dependent upon the condition to be treated. Examples of treatments and therapies include, but are not limited to, chemotherapy (the administration of active agents, including, e.g. drugs, such as chemotherapeutics); surgery; and radiation therapy.

AXL Inhibitors (AXLi)

Small Molecule AXL Inhibitors

General Formula

In some embodiments the AXL inhibitor is a compound of formula (I):

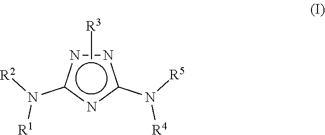

wherein:
R¹, R⁴ and R⁵ are each independently selected from the group consisting of hydrogen, alkyl, alkenyl, aryl, aralkyl, —C(O)R⁸, —C(O)N(R⁶)R⁷, and —C(═NR⁶)N(R⁶)R⁷;
R² and R³ are each independently a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR⁸, —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2);
or R² is a polycyclic heteroaryl containing more than 14 ring atoms as described above and R³ is selected from the group consisting of aryl and heteroaryl, where the aryl and the heteroaryl are each independently optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2);
or R³ is a polycyclic heteroaryl containing more than 14 ring atoms as described above, and R² is selected from the group consisting of aryl and heteroaryl, where the aryl and the heteroaryl are each independently optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2);
each R⁶ and R⁷ is independently selected from the group consisting of hydrogen, alkyl, alkenyl, alkynyl, haloalkyl, haloalkenyl, haloalkynyl, hydroxyalkyl, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, alkenyl, alkynyl, haloalkyl, haloalkenyl, haloalkynyl, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, and optionally substituted heteroarylalkynyl;
each R⁹ is independently selected from the group consisting of a direct bond, an optionally substituted straight or branched alkylene chain, an optionally substituted straight or branched alkenylene chain and an optionally substituted straight or branched alkynylene chain;
each R¹⁰ is independently selected from the group consisting of an optionally substituted straight or branched alkylene chain, an optionally substituted straight or branched alkenylene chain and an optionally substituted straight or branched alkynylene chain;
each R¹¹ is independently selected from the group consisting of hydrogen, alkyl, cyano, nitro and —OR⁸—;
each R¹² is independently selected from the group consisting of hydrogen, alkyl, alkenyl, haloalkyl, optionally substituted cycloalkyl, optionally substituted cycloalkylalkyl, optionally substituted aryl, optionally substituted aralkyl, optionally substituted heterocyclyl, optionally substituted

heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or two R^{12's}, together with the common nitrogen to which they are both attached, form an optionally substituted N-heterocyclyl or an optionally substituted N-heteroaryl;
each R¹³ is independently selected from the group consisting of a direct bond, an optionally substituted straight or branched alkylene chain and an optionally substituted straight or branched alkenylene chain; and
each R¹⁴ is independently selected from the group consisting of an optionally substituted straight or branched alkylene chain and an optionally substituted straight or branched alkenylene chain;
as an isolated stereoisomer or mixture thereof or as a tautomer or mixture thereof, or a pharmaceutically acceptable salt or N-oxide thereof.

### Some embodiments

In some embodiments, the compound of formula (I) is a compound of formula (Ia):

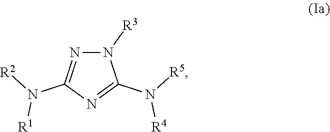

wherein R¹, R², R³, R⁴ and R⁵ are as described above for compounds of formula (I), as an isolated stereoisomer or mixture thereof or as a tautomer or mixture thereof, or a pharmaceutically acceptable salt or N-oxide thereof.

In some embodiments in the compound of formula (Ia) as set forth above, R² and R³ are each independently a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl,

optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)O R¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); and R¹, R⁴, R⁵, each R⁶, each R⁷, each R⁸, each R⁹, each R¹⁰, each R¹¹ and R¹² are as described above for compounds of formula (Ia).

In other embodiments, in the compound of formula (Ia) as set forth above:
R¹, R⁴ and R⁵ are each hydrogen;
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally

substituted heterocyclylalkyl, optionally substituted heteroaryl, optionally substituted heteroaryl alkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
each R⁹ is independently selected from the group consisting of a direct bond and an optionally substituted straight or branched alkylene chain;
each R¹⁰ is an optionally substituted straight or branched alkylene chain; and
each R¹¹ is independently selected from the group consisting of hydrogen, alkyl, cyano, nitro and —OR⁸.

In some embodiments in the compound of formula (Ia) as set forth above:
R² and R³ are each independently a polycyclic heteroaryl containing more than 14 ring atoms selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl,

optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments in the compound of formula (Ia) is 1-(6,7-dihydro-5H-benzo[6,7]cyclohepta[1,2-c]pyridazin-3-yl)-N³-(5′,5′-dimethyl-6,8,9,10-tetrahydro-5H-spiro[cycloocta[b]pyridine-7,2′-[1,3]dioxane]-3-yl)-1H-1,2,4-triazole-3,5-diamine.

In some embodiments in the compound of formula (Ia) as set forth above, R² is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally

substituted heteroaryl, optionally substituted heterocyclyl, —R9—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or

2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); R³ is selected from the group consisting of aryl and heteroaryl, where the aryl and the heteroaryl are each independently optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally

substituted heteroarylalkenyl, optionally substituted heteroarylalkynyl, —R¹³—OR¹², —R¹³—OC(O)—R¹²—, —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and R¹, R⁴, R⁵, each R⁶, each R⁷, each R⁸, each R⁹, each R¹⁰, each R¹¹, each R¹², each R¹³ and each R¹⁴ are as described above for compounds of formula (Ia).

In some embodiments in the compound of formula (Ia) as set forth above:
R¹, R⁴ and R⁵ are each hydrogen;
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally

substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
each R⁹ is independently selected from the group consisting of a direct bond and an optionally substituted straight or branched alkylene chain;

each R¹⁰ is an optionally substituted straight or branched alkylene chain;
each R¹¹ is independently selected from the group consisting of hydrogen, alkyl, cyano, nitro and —OR⁸;
each R¹² is independently selected from the group consisting of hydrogen, alkyl, alkenyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{12's}, together with the common nitrogen to which they are both attached, form an optionally substituted N-heterocyclyl or an optionally substituted N-heteroaryl;
each R¹³ is independently selected from the group consisting of a direct bond and an optionally substituted straight or branched alkylene chain; and
each R¹⁴ is an optionally substituted straight or branched alkylene chain.

In other embodiments, in the compound of formula (Ia) as set forth above:
R¹, R⁴ and R⁵ are each hydrogen;
R² is a polycyclic heteroaryl containing more than 14 ring atoms selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl,

optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2);
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally

substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
each R⁹ is independently selected from the group consisting of a direct bond and an optionally substituted straight or branched alkylene chain;
each R¹⁰ is an optionally substituted straight or branched alkylene chain;
each R¹² is independently selected from the group consisting of hydrogen, alkyl, alkenyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{12's}, together with the common nitrogen to which they are both attached, form an optionally substituted N-heterocyclyl or an optionally substituted N-heteroaryl;
each R¹³ is independently selected from the group consisting of a direct bond and an optionally substituted straight or branched alkylene chain; and
each R¹⁴ is an optionally substituted straight or branched alkylene chain.

In some embodiments in the compound of formula (Ia) as set forth above:
R² is a polycyclic heteroaryl containing more than 14 ring atoms selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); and
R³ is heteroaryl selected from the group consisting of pyridinyl, pyrimidinyl, 4,5-dihydro-1H-benzo[b]azepin-2(3H)-on-8-yl, benzo[d]imidazolyl, 6,7,8,9-tetrahydro-5H-pyrido[3,2-d]azepin-3-yl, 6,7,8,9-tetrahydro-5H-pyrido[3,2-c]azepin-3-yl, 5,6,7,8-tetrahydro-1,6-naphthyridin-3-yl, 5,6,7,8-tetrahydroquinolin-3-yl, 1,2,3,4-tetrahydroisoquinolin-7-yl, 2,3,4,5-tetrahydrobenzo[b]oxepin-7-yl, 3,4-dihydro-2H-benzo[b][1,4]dioxepin-7-yl, benzo[d]oxazol-5-yl, 3,4-dihydro-2H-benzo[b][1,4]oxazin-7-yl, benzo[b]thiophenyl, thieno[3,2-d]pyrimidinyl and 6,7,8,9-tetrahydro-5H-cyclohepta[b]pyridin-3-yl, each optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted

heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2).

In some embodiments, the compound of formula (Ia), as set forth above, is selected from the group consisting of:
1-(6,7-dimethoxy-quinazolin-4-yl)-N³-(5,7,8,9-tetrahydrospiro[cyclohepta[b]pyridine-6,2′-[1,3]dioxolane]-3-yl)-1H-1,2,4-triazole-3,5-diamine;
1-(2-chloro-7-methylthieno[3,2-d]pyrimidin-4-yl)-N³-(5,7,8,9-tetrahydrospiro[cyclohepta[b]pyridine-6,2′-[1,3]dioxolane]-3-yl)-1H-1,2,4-triazole-3,5-diamine;
1-(2-chloro-7-methylthieno[3,2-d]pyrimidin-4-yl)-N³-(5,6,8,9-tetrahydrospiro[cyclohepta[b]pyridine-7,2′-[1,3]dioxolane]-3-yl)-1H-1,2,4-triazole-3,5-diamine; and
1-(2-chloro-7-methylthieno[3,2-d]pyrimidin-4-yl)-N³-(5′,5′-dimethyl-6,8,9,10-9tetrahydro-5H-spiro[cycloocta[b]pyridine-7,2′-[1,3]dioxane]-3-yl)-1H-1,2,4-triazole-3,5-diamine.

In some embodiments in the compound of formula (Ia) as set forth above, R² is selected from the group consisting of aryl and heteroaryl, where the aryl and the heteroaryl are each independently optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); R³ is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl,

optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); and R¹, R⁴, R⁵, each R⁶, each R⁷, each R⁸, each R⁹, each R¹⁰, each R¹¹, each R¹², each R¹³ and each R¹⁴ are as described above for compounds of formula (I).

In some embodiments in the compound of formula (Ia) as set forth above:
R¹, R⁴ and R⁵ are each independently hydrogen;
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally

substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, alkenyl, alkynyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
each R⁹ is independently selected from the group consisting of a direct bond and an optionally substituted straight or branched alkylene chain;
each R¹⁰ is an optionally substituted straight or branched alkylene chain;
each R¹¹ is independently selected from the group consisting of hydrogen, alkyl, cyano, nitro and —OR⁸;
each R¹² is independently selected from the group consisting of hydrogen, alkyl, alkenyl, haloalkyl, optionally substituted cycloalkyl, optionally substituted cycloalkylalkyl, optionally substituted aryl, optionally substituted aralkyl, optionally substituted heterocyclyl, optionally substituted

heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or two R^{12's}, together with the common nitrogen to which they are both attached, form an optionally substituted N-heterocyclyl or an optionally substituted N-heteroaryl;
each R¹³ is independently selected from the group consisting of a direct bond and an optionally substituted straight or branched alkylene chain; and
each R¹⁴ is an optionally substituted straight or branched alkylene chain.

In some embodiments in the compound of formula (Ia) as set forth above:
R² is aryl optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally

substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)2 (where t is 1 or 2).

In some embodiments in the compound of formula (Ia) as set forth above:
R² is aryl selected from the group consisting of phenyl and 6,7,8,9-tetrahydro-5H-benzo[7]annulene-2-yl, each optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally

substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)-R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl,

optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments in the compound of formula (Ia) as set forth above:
R² is phenyl optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally

substituted heteroarylalkenyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2).

In some embodiments in the compound of formula (Ia) as set forth above:
R² is phenyl optionally substituted by one or more substituents selected from the group consisting of alkyl, halo, haloalkyl, cyano, and optionally substituted heterocyclyl where the optionally substituted heterocyclyl is selected from the group consisting of piperidinyl, piperazinyl, pyrrolidinyl, azepanyl, decahydropyrazino[1,2-a]azepinyl, octahydropyrrolo[3,4-c]pyrrolyl, azabicyclo[3.2.1]octyl, octahydropyrrolo[3,4-b]pyrrolyl, octahydropyrrolo[3,2-c]pyridinyl, 2,7-diazaspiro[4.4]nonanyl and azetidinyl; each independently optionally substituted by one or two substituents selected from the group

consisting of —R⁹—OR⁸, —R⁹—N(R⁶)R⁷, —R⁹—C(O)OR⁶, —R⁹—C(O)N(R⁶)R⁷, —R⁹—N(R⁶)C(O)R⁷, —R⁹—N(R⁶)C(O)OR⁷, alkyl, halo, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
R³ is selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7-dihydro-5H-benzo[6,7]cyclohepta[1,2-b]pyridin-2-yl, 6,7-dihydro-5H-benzo[2,3]thiepino[4,5-c]pyridazin-3-yl, spiro[chromeno[4,3-c]pyridazine-5,1′-cyclopentane]-3-yl and 6,7-dihydro-5H-benzo[6,7]cyclohepta[4,5-c]pyridazin-3-yl, each optionally substituted by one or more substituents selected from the group consisting of alkyl, aryl, halo and —R⁹—OR⁸.

In some embodiments the compound of formula (Ia), as set forth above, is selected from the group consisting of:
N³-(4-(4-cyclohexanylpiperazin-1-yl)phenyl)-1-(6,7-dihydro-5H-benzo[6,7]cyclohepta[1,2-c]pyridazin-3-yl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(pyrrolidin-1-yl)piperidin-1-yl)-1H-1,2,4-triazole-3,5-diamine;
1-(6,7-dihydro-5H-benzo[6,7]cyclohepta[1,2-c]pyridazin-3-yl)-N³-(3-fluoro-4-(4-methyl-3-phenylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-(4-(4-piperidin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(indolin-2-on-1-yl)piperidin-1₁1)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(morpholin-4-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(4-cyclopentyl-2-methylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(3,5-dimethylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(4-(pyrrolidin-1-yl)piperidin-1-yl)-3-cyanophenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-(diethylamino)pyrrolidin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(4-(bicyclo[2.2.1]heptan-2-yl)piperazin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(4-methylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(diethylamino)piperidin-1-yl)phenyl)-1H-1,2,4-triazole-3,5-diamine;
1-(6,7-dihydro-5H-9-methoxybenzo[6,7]cyclohepta[1,2-c]pyridazin-3-yl)-N3-(3-fluoro-4-(4-(pyrrolidin-1-yl)piperdin-1-yl)phenyl)-1H-1,2,4-triazole-3,5-diamine;
1-(6,7-dihydro-5H-10-fluorobenzo[6,7]cyclohepta[1,2-c]pyridazin-3-yl)-N³-(3-fluoro-4-(4-(pyrrolidin-1-yl)piperdin-1-yl)phenyl)-1H-1,2,4-triazole-3,5-diamine;
1-(6,7-dihydro-5H-10-fluorobenzo[6,7]cyclohepta[1,2-c]pyridazin-3-yl)-N³-(3-fluoro-4-(4-(cyclohexyl)piperazin-1-yl)phenyl)-1H-1,2,4-triazole-3,5-diamine;
1-(6,7-dihydro-5H-9-methoxybenzo[6,7]cyclohepta[1,2-c]pyridazin-3-yl)-N3-(3-fluoro-4-(4-(cyclohexyl)piperazin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(4-methylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(4-methylpiperidin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-dimethylaminopiperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-chloro-4-(4-pyrrolidin-1-yl-piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-trifluoromethyl-4-(4-pyrrolidin-1-yl-piperidin-1-yl)phenyl)-1H-1,2,4-triazole-3,5-diamine;
1-(6,7-dihydro-5H-9,10-dimethoxybenzo[6,7]cyclohepta[1,2-c]pyridazin-3-yl)-N³-(3-fluoro-4-(4-pyrrolidin-1-yl-piperidin-1-yl)phenyl)-1H-1,2,4-triazole-3,5-diamine;
1-(6,7-dihydro-5H-9,10,11-trimethoxybenzo[6,7]cyclohepta[1,2-c]pyridazin-3-yl)-N³-(3-fluoro-4-(4-pyrrolidin-1-yl-piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(5-methyloctahydropyrrolo[3,4-c]pyrrolyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-pyrrolidin-1-yl-piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-pyrrolidin-1-yl-azepan-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-N-methylpiperidin-4-yl-piperidin-1-yl)phenyl)-1H-1,2,4-triazole-3,5-diamine; 1-(6,7-dihydro-5H-benzo[6,7]cyclohepta[4,5-c]pyridazin-2-yl)-N³-(3-fluoro-4-(4-(pyrrolidinyl)piperidinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(5-propyloctahydropyrrolo[3,4-c]pyrrolyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(decahydropyrazino[1,2-a]azepin-2-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(5-cyclopentyloctahydropyrrolo[3,4-c]pyrrolyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-(pyrrolidin-1-yl)-8-azabicyclo[3.2.1]oct-8-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-pyrrolidin-1-yl-azepan-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(4-methylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(4-isopropylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(1-methyloctahydropyrrolo[3,4-b]pyrrol-5-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(N-methylcyclopentylamino)piperidinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(dipropylamino)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(1-propyloctahydro-1H-pyrrolo[3,2-c]pyridine-5-yl)phenyl)-1H-1,2,4-triazole-3,5-diamine;
1-(6,7-dihydro-5H-benzo[6,7]cyclohepta[4,5-c]pyridazin-2-yl)-N³-(3-fluoro-4-(4-(N-methylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(tert-butyloxycarbonylamino)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-aminopiperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(5-cyclohexyloctahydropyrrolo[3,4-c]pyrrolyl)piperidin-1-yl)phenyl) -1H-1,2,4-triazole-3,5-diamine;
1-(6,7-dihydro-5H-benzo[6,7]cyclohepta[1,2-c]pyridazin-3-yl)-N³-(4-(methylpiperidin-4-yl)phenyl)-1H-1,2,4-triazole-3,5-diamine;
1-(6,7-dihydro-5H-benzo[6,7]cyclohepta[1,2-c]pyridazin-3-yl)-N³-(4-(4-pyrrolidin-1-ylpiperidinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-pyrrolidin-1-ylpiperidinyl)phenyl)-1H-1,2,4-triazole-3,5-diamine;
1-(6,7-dihydro-5H-benzo[6,7]cyclohepta[1,2-c]pyridazin-3-yl)-N³-(3-methyl-4-(4-pyrrolidin-1-ylpiperidinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-cyclopentylpiperazinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-N-methylpiperidin-4-ylpiperazinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(7-methyl-2,7-diazaspiro[4.4]nonan-2-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(N-isopropylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-pyrrolidin-1-ylazetidinyl)phenyl)-1H-1,2,4-triazole-3,5-diamine;
1-(6,7-dihydro-5H-benzo[6,7]cyclohepta[1,2-c]pyridazin-3-yl)-N³-(3-methyl-4-(4-(N-methylpiperazin-4-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N3-(3-fluoro-4-((S)-3-(pyrrolidin-1-ylmethyl)pyrrolidinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(pyrrolidinylmethyl)piperidinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-((4aR,8aS)-decahydroisoquinolin-2-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(octahydro-1H-pyrido[1,2-a]pyrazin-2-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-(4-(3-pyrrolidin-1-yl)pyrrolidin-1-yl)phenyl) -1H-1,2,4-triazole-3,5-diamine;
1-(6,7-dihydro-5H-benzo[6,7]cyclohepta[1,2-c]pyridazin-3-yl)-N³-(3-fluoro-4-(4-(5-methyloctahydropyrrolo[3,4-c]pyrrolyl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(octahydropyrrolo[3,4-c]pyrrolyl)phenyl)-1H-1,2,4-triazole-3,5-diamine;
1-(6,7-dihydro-9-chloro-5H-benzo[6,7]cyclohepta[1,2-c]pyridazin-3-yl)-N³-(3-fluoro-4-(4-pyrrolidin-1-ylpiperidin-1-yl)phenyl)-1H-1,2,4-triazole-3,5-diamine;
1-(6,7-dihydro-9-chloro-5H-benzo[6,7]cyclohepta[1,2-c]pyridazin-3-yl)-N³-(3-fluoro-4-(4-(N-methylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(4-iodophenyl)-1H-1,2,4-triazole-3,5-diamine;
1-(spiro[chromeno[4,3-c]pyridazine-5,1′-cyclopentane]-3-yl)-N³-(3-fluoro-4-(4-(4-methylpiperazin-1-yl)piperidin-1-yl)phenyl)-1H-1,2,4-triazole-3,5-diamine;
1-(spiro[chromeno[4,3-c]pyridazine-5,1′-cyclopentane]-3-yl)-N³-(3-fluoro-4-(4-(pyrrolidin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-pyrrolidin-1-ylpiperidin-1-yl)phenyl)-1H-1,2,4-triazole-3,5-diamine;
1-(6,7-dihydro-5H-benzo[6,7]cyclohepta[4,5-c]pyridazin-2-yl)-N³-(3-fluoro-4-(4-pyrrolidin-1-ylpiperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(4-methylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-(3R)-dimethylaminopyrrolidin-1-yl)phenyl)-1H-1,2,4-triazole-3,5-diamine;
1-(6,7-dihydro-5H-benzo[6,7]cyclohepta[4,5-c]pyridazin-2-yl)-N³-(3-methyl-4-(4-pyrrolidin-1-ylpiperidin-1-yl)phenyl)-1H-1,2,4-triazole-3,5-diamine;
1-(6,7-dihydro-5H-benzo[6,7]cyclohepta[4,5-c]pyridazin-2-yl)-N³-(3-fluoro-4-(4-pyrrolidin-1-ylpiperidin-1-yl)phenyl)-1H-1,2,4-triazole-3,5-diamine;
1-(4-phenyl-6,7-dihydro-5H-benzo[6,7]cyclohepta[1,2-b]pyridin-2-yl)-N³-(3-fluoro-4-(4-cyclohexylpiperazin-1-yl)phenyl) -1H-1,2,4-triazole-3,5-diamine;
1-(4-phenyl-6,7-dihydro-5H-benzo[6,7]cyclohepta[1,2-b]pyridin-2-yl)-N³-(4-(4-methylpiperazin-1-yl)phenyl) -1H-1,2,4-triazole-3,5-diamine;
1-(6,7-dihydro-5H-benzo[6,7]cyclohepta[1,2-c]pyridazin-3-yl)-N³-(3-fluoro-4-(4-methylpiperazin-1-yl)phenyl) -1H-1,2,4-triazole-3,5-diamine;
1-(6,7-dihydro-5H-benzo[6,7]cyclohepta[1,2-c]pyridazin-3-yl)-N³-(4-(1-bicyclo[2.2.1]heptan-2-yl)-piperidin-4-ylphenyl)-1H-1,2,4-triazole-3,5-diamine;
1-(6,7-dihydro-5H-benzo[6,7]cyclohepta[1,2-c]pyridazin-3-yl)-N³-(4-(1-cyclopropylmethylpiperidin-4-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-cyclopropylmethylpiperazin-1-yl)phenyl)-1H-1,2,4-triazole-3,5-diamine;
1-(6,7-dihydro-5H-benzo[2,3]thiepino[4,5-c]pyridazin-3-yl)-N³-(4-(1-bicyclo[2.2.1 ]heptan-2-yl)-piperidin-4-ylphenyl)-1H-1,2,4-triazole-3,5-diamine;
1-(4-phenyl-6,7-dihydro-5H-benzo[6,7]cyclohepta[1,2-b]pyridin-2-yl)-N³-(3-fluoro-4-(4-pyrrolidin-1-ylpiperidin-1-yl)phenyl)-1H-1,2,4-triazole-3,5-diamine, and
1-(6,7-dihydro-5H-pyrido[2′,3′:6,7]cyclohepta[1,2-c]pyridazin-3-yl)-N³-(3-fluoro-4-(4-(pyrrolidin-1-yl)piperidin-1-yl)phenyl)-1H-1,2,4-triazole-3,5-diamine.

In some embodiments in the compound of formula (Ia) as set forth above:
R² is phenyl optionally substituted by one or more substituents selected from the group consisting of halo, alkyl, heterocyclylalkenyl, —R¹³—OR¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)N(R¹²)₂, and —R¹³—N(R¹²)C(O)R¹²;
R³ is selected from the group consisting of 6,7-dihydro-5H-benzo[6,7]cyclohepta[1,2-c]pyridazin-3-yl and 6,7-dihydro-5H-benzo[6,7]cyclohepta[4,5-c]pyridazin-3-yl, each optionally substituted by one or more substituents selected from the group consisting of alkyl, aryl, halo and —R⁹—OR⁸.

In some embodiments the compound of formula (Ia), as set forth above, is selected from the group consisting of:
1-(6,7-dihydro-5H-benzo[6,7]cyclohepta[1,2-c]pyridazin-3-yl)-N³-(4-(2-(pyrrolidin-1-yl)ethoxy)phenyl)-1H-1,2,4-triazole-3,5-diamine;
1-(6,7-dihydro-5H-benzo[6,7]cyclohepta[1,2-c]pyridazin-3-yl)-N³-(4-(4-(cyclopentyl)piperazin-1-ylcarbonyl)phenyl)-1H-1,2,4-triazole-3,5-diamine;
1-(6,7-dihydro-5H-benzo[6,7]cyclohepta[1,2-c]pyridazin-3-yl)-N³-(4-((2-pyrrolidin-1-ylethyl)aminocarbonyl)phenyl)-1H-1,2,4-triazole-3,5-diamine;
1-(6,7-dihydro-5H-benzo[6,7]cyclohepta[1,2-c]pyridazin-3-yl)-N³-(4-(2,2,6,6-tetramethylpiperidin-1-yl)ethoxyphenyl)-1H-1,2,4-triazole-3,5-diamine;
1-(6,7-dihydro-5H-benzo[6,7]cyclohepta[1,2-c]pyridazin-3-yl)-N³-(4-((2-(dimethylamino)ethyl)aminocarbonyl)phenyl)-1H-1,2,4-triazole-3,5-diamine;
1-(6,7-dihydro-5H-benzo[6,7]cyclohepta[1,2-c]pyridazin-3-yl)-N³-(4-((2-(methoxy)ethyl)aminocarbonyl)phenyl)-1H-1,2,4-triazole-3,5-diamine;
1-(6,7-dihydro-5H-benzo[6,7]cyclohepta[1,2-c]pyridazin-3-yl)-N3-(4-((2-(pyrrolidin-1-yl)ethyl)aminocarbonyl)phenyl)-1H-1,2,4-triazole-3,5-diamine;
1-(6,7-dihydro-5H-benzo[6,7]cyclohepta[1,2-c]pyridazin-3-yl)-N3-(4-((4-(pyrrolidin-1-yl)piperidin-1-yl)carbonyl)phenyl)-1H-1,2,4-triazole-3,5-diamine;
1-(6,7-dihydro-5H-benzo[6,7]cyclohepta[1,2-c]pyridazin-3-yl)-N³-(3-chloro-4-(2-(pyrrolidin-1-yl)ethoxy)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(2-(pyrrolidin-1-yl)ethoxy)phenyl)-1H-1,2,4-triazole-3,5-diamine;
1-(6,7-dihydro-5H-10-fluorobenzo[6,7]cyclohepta[1,2-c]pyridazin-3-yl)-N³-(3-fluoro-4-(2-(pyrrolidin-1-yl)ethoxy)phenyl)-1H-1,2,4-triazole-3,5-diamine;
1-(6,7-dihydro-5H-9-methoxybenzo[6,7]cyclohepta[1,2-c]pyridazin-3-yl)-N³-(3-fluoro-4-(2-(pyrrolidin-1-yl)ethoxy)phenyl)-1H-1,2,4-triazole-3,5-diamine;
1-(6,7-dihydro-5H-benzo[6,7]cyclohepta[1,2-c]pyridazin-3-yl)-N³-(4-(2-(N-methylcyclopentylamino)ethoxy)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(N-methylpiperidin-4-yl-N-methylamino)phenyl)-1H-1,2,4-triazole-3,5-diamine;
1-(6,7-dihydro-5H-benzo[6,7]cyclohepta[1,2-c]pyridazin-3-yl)-N³-(4-((N-butyl-N-acetoamino)methyl)phenyl)-1H-1,2,4-triazole-3,5-diamine;
1-(6,7-dihydro-5H-benzo[6,7]cyclohepta[1,2-c]pyridazin-3-yl)-N³-(4-(4-(4-methylpiperazin-1-yl)piperidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(4-(piperidin-1-yl)piperidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(piperidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(pyrrolidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(3-dimethylaminopyrrolidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(3-diethylaminopyrrolidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(4-pyrrolidin-1-ylpiperidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(4-methylpiperazin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(4-isopropylpiperazin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(4-cyclopentylpiperazin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(morpholin-4-ylprop-1-enyl)phenyl)-1H-1,2,4-triazole-3,5-diamine; and
1-(6,7-dihydro-5H-benzo[6,7]cyclohepta[1,2-c]pyridazin-3-yl)-N³-(4-(1-methylpiperidin-3-yl-oxy)phenyl)-1H-1,2,4-triazole-3,5-diamine.

In some embodiments in the compound of formula (Ia) as set forth above:
R² is phenyl optionally substituted by one or more substituents selected from the group consisting of alkyl, halo, haloalkyl, cyano, and optionally substituted heterocyclyl where the optionally substituted heterocyclyl is selected from the group consisting of piperidinyl, piperazinyl, pyrrolidinyl, azepanyl, decahydropyrazino[1,2-a]azepinyl, octahydropyrrolo[3,4-c]pyrrolyl, azabicyclo[3.2.1]octyl, octahydropyrrolo[3,4-b]pyrrolyl, octahydropyrrolo[3,2-c]pyridinyl, 2,7-diazaspiro[4.4]nonanyl and azetidinyl; each independently optionally substituted by one or two substituents selected from the group

consisting of —R⁹—OR⁸, —R⁹—N(R⁶)R⁷, —R⁹—C(O)OR⁶, —R⁹—C(O)N(R⁶)R⁷, —R⁹—N(R⁶)C(O)R⁷, —R⁹—N(R⁶)C(O)OR⁷, alkyl, halo, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl; and
R³ is selected from the group consisting of 6,7-dihydro-5H-benzo[6,7]cyclohepta[1,2-d]pyrimidin-4-yl and 6,7-dihydro-5H-benzo[6,7]cyclohepta[1,2-d]pyrimidin-2-yl, each optionally substituted by one or more substituents selected from the group consisting of alkyl, aryl, halo and —R⁹—OR⁸.

In some embodiments the compound of formula (Ia), as set forth above, is selected from the group consisting of:
1-(6,7-dihydro-5H-benzo[6,7]cyclohepta[1,2-d]pyrimidin-4-yl)-N³-(4-(4-(bicyclo[2.2.1]heptan-2-yl)piperazin-1-yl)phenyl)-1H-1,2,4-triazole-3,5-diamine;
1-(6,7-dihydro-5H-benzo[6,7]cyclohepta[1,2-d]pyrimidin-4-yl)-N³-(3-fluoro-4-(4-(diethylamino)piperidin-1-yl)phenyl)-1H-1,2,4-triazole-3,5-diamine;
1-(6,7-dihydro-5H-benzo[6,7]cyclohepta[1,2-d]pyrimidin-2-yl)-N³-(4-(N-methylpiperazin-1-yl)phenyl)-1H-1,2,4-triazole-3,5-diamine;
1-(6,7-dihydro-5H-benzo[6,7]cyclohepta[1,2-d]pyrimidin-2-yl)-N³-(3-fluoro-4-(4-cyclohexylpiperazinyl)phenyl)-1H-1,2,4-triazole-3,5-diamine; and
1-(6,7-dihydro-5H-benzo[6,7]cyclohepta[1,2-d]pyrimidin-2-yl)-N³-(4-(4-(2S)-bicyclo[2.2.1]heptan-2-yl)-piperazinylphenyl)-1H-1,2,4-triazole-3,5-diamine.

In some embodiments in the compound of formula (Ia) as set forth above:
R² is phenyl optionally substituted by one or more substituents selected from the group consisting of halo, alkyl, heterocyclylalkenyl, —R¹³—OR¹², —R¹³—O—R¹⁴—N(R¹²)₂, —R13—N(R12)-R14_N(R¹²)₂, —R¹³—N(R¹²)₂, —R¹³—C(O)R¹², —R¹³—C(O)N(R¹²)₂, and —R¹³—N(R¹²)C(O)R¹²; and
R³ is selected from the group consisting of 6,7-dihydro-5H-benzo[6,7]cyclohepta[1,2-d]pyrimidin-4-yl and 6,7-dihydro-5H-benzo[6,7]cyclohepta[1,2-d]pyrimidin-2-yl, each optionally substituted by one or more substituents selected from the group consisting of alkyl, aryl, halo and —R⁹—OR⁸.

In some embodiments the compound of formula (Ia), as set forth above, is selected from the group consisting of:
1-(6,7-dihydro-5H-benzo[6,7]cyclohepta[1,2-d]pyrimidin-2-yl)-N³-(3-fluoro-4-(2-(pyrrolidin-1-yl)ethoxy)phenyl)-1H-1,2,4-triazole-3,5-diamine; and
1-(6,7-dihydro-5H-benzo[6,7]cyclohepta[1,2-d]pyrimidin-4-yl)-N3-(4-(2-(pyrrolidin-1-yl)ethoxy)phenyl)-1H-1,2,4-triazole-3,5-diamine.

In some embodiments in the compound of formula (Ia) as set forth above:
R² is phenyl optionally substituted by one or more substituents selected from the group consisting of alkyl, halo, haloalkyl, cyano, and optionally substituted heterocyclyl where the optionally substituted heterocyclyl is selected from the group consisting of piperidinyl, piperazinyl, pyrrolidinyl, azepanyl, decahydropyrazino[1,2-a]azepinyl, octahydropyrrolo[3,4-c]pyrrolyl, azabicyclo[3.2.1]octyl, octahydropyrrolo[3,4-b]pyrrolyl, octahydropyrrolo[3,2-c]pyridinyl, 2,7-diazaspiro[4.4]nonanyl and azetidinyl; each independently optionally substituted by one or two substituents selected from the group

consisting of —R⁹—OR⁸, —R⁹—N(R⁶)R⁷, —R⁹—C(O)OR⁸, —R⁹—C(O)N(R⁶)R⁷, —R⁹—N(R⁶)C(O)R⁷, —R⁹—N(R⁶)C(O)OR⁷, alkyl, halo, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl; and
R³ is selected from the group consisting of 6,7-dihydro-SH-benzo[2,3]azepino[4,5-c]pyridazin-3-yl, (Z)-dibenzo[b,f][1,4]thiazepin-11-yl, 6,7-dihydro-5H-benzo[2,3]oxepino[4,5-c]pyridazin-3-yl, and 6,7-dihydro-5H-benzo[2,3]thiepino[4,5-c]pyridazin-3-yl, each optionally substituted by one or more substituents selected from the group consisting of alkyl, aryl, halo and —R⁹—OR⁸.

In some embodiments the compound of formula (Ia), as set forth above, is selected from the group consisting of:
1-(7-methyl-6,7-dihydro-5H-benzo[2,3]azepino[4,5-c]pyridazin-3-yl)-N³-(4-(N-methylpiperazin-1-yl)phenyl)-1H-1,2,4-triazole-3,5-diamine;
1-(7-methyl-6,7-dihydro-5H-benzo[2,3]azepino[4,5-c]pyridazin-3-yl)-N³-(3-fluoro-4-(4-cyclohexylpiperazinyl)phenyl)-1H-1,2,4-triazole-3,5-diamine;
1-((Z)-dibenzo[b,f][1,4]thiazepin-11-yl)-N³-(4-(4-N-methylpiperazinyl)phenyl)-1H-1,2,4-triazole-3,5-diamine;
1-((Z)-dibenzo[b,f][1,4]thiazepin-11-yl)-N³-(3-fluoro-4-(4-diethylaminopiperidin-1-yl)phenyl)-1H-1,2,4-triazole-3,5-diamine;
1-(6,7-dihydro-5H-benzo[2,3]oxepino[4,5-c]pyridazin-3-yl)-N³-(4-(4-pyrrolidin-1-ylpiperidinyl)phenyl)-1H-1,2,4-triazole-3,5-diamine;
1-(6,7-dihydro-5H-benzo[2,3]oxepino[4,5-c]pyridazin-3-yl)-N3-(3-fluoro-4-(4-pyrrolidin-1-ylpiperidinyl)phenyl)-1H-1,2,4-triazole-3,5-diamine;
1-(6,7-dihydro-5H-benzo[2,3]thiepino[4,5-c]pyridazin-3-yl)-N3-(3-fluoro-4-(4-pyrrolidin-1-ylpiperidinyl)phenyl)-1H-1,2,4-triazole-3,5-diamine;
1-(6,7-dihydro-5H-benzo[2,3]thiepino[4,5-c]pyridazin-3-yl)-N3-(4-(4-pyrrolidin-1-ylpiperidinyl)phenyl)-1H-1,2,4-triazole-3,5-diamine;
1-(6,7-dihydro-5H-benzo[2,3]thiepino[4,5-c]pyridazin-3-yl)-N³-(3-fluoro-4-(4-(pyrrolidinylmethyl)piperidinyl)phenyl)-1H-1,2,4-triazole-3,5-diamine;
1-(6,7-dihydro-5H-benzo[2,3]thiepino[4,5-c]pyridazin-3-yl)-N³-(3-fluoro-4-((4aR,8aS)-decahydroisoquinolin-2-yl)phenyl)-1H-1,2,4-triazole-3,5-diamine; and
1-(6,7-dihydro-5H-benzo[2,3]thiepino[4,5-c]pyridazin-3-yl)-N³-(3-fluoro-4-(octahydro-1H-pyrido[1,2-a]pyrazin-2-yl)phenyl)-1H-1,2,4-triazole-3,5-diamine.

In some embodiments in the compound of formula (Ia) as set forth above:
R² is phenyl optionally substituted by one or more substituents selected from the group consisting of halo, alkyl, heterocyclylalkenyl, —R¹³—OR¹², —R¹³—O—R¹⁴—N(R¹²)₂, —R¹³—N(R¹²)-R¹⁴—N(R¹²)₂, —R¹³—N(R¹²)₂, —R¹³—C(O)R¹², —R¹³—C(O)N(R¹²)₂, and —R¹³—N(R¹²)C(O)R¹²; and
R³ is selected from the group consisting of 6,7-dihydro-5H-benzo[2,3]azepino[4,5-c]pyridazin-3-yl, (Z)-dibenzo[b,f][1,4]thiazepin-11-yl, 6,7-dihydro-5H-benzo[2,3]oxepino[4,5-c]pyridazin-3-yl, and 6,7-dihydro-5H-benzo[2,3]thiepino[4,5-c]pyridazin-3-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl,

optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸

(where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments the compound of formula (Ia), as set forth above, is selected from the group consisting of:
1-(7-methyl-6,7-dihydro-5H-benzo[2,3]azepino[4,5-c]pyridazin-3-yl)-N³-(3-fluoro-4-(2-(pyrrolidin-1-yl)ethoxy)phenyl)-1H-1,2,4-triazole-3,5-diamine; and
1-((Z)-dibenzo[b,f][1,4]thiazepin-11-yl)-N³-(4-(2-(pyrrolidin-1-yl)ethoxy)phenyl)-1H-1,2,4-triazole-3,5-diamine.

In some embodiments in the compound of formula (Ia) as set forth above:
R² is phenyl optionally substituted by a substituent selected from the group consisting of optionally substituted heterocyclylalkyl, optionally substituted heteroaryl and optionally substituted heteroarylalkyl;
R³ is selected from the group consisting of 6,7-dihydro-5H-benzo[6,7]cyclohepta[1,2-c]pyridazin-3-yl and 6,7-dihydro-5H-benzo[2,3]thiepino[4,5-c]pyridazin-3-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl,

optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2);
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally

substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁵—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, alkenyl, alkynyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
each R⁹ is independently selected from the group consisting of a direct bond and an optionally substituted straight or branched alkylene chain;
each R¹⁰ is an optionally substituted straight or branched alkylene chain; and

R¹² is independently selected from the group consisting of hydrogen, alkyl, haloalkyl, alkenyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl.

In some embodiments the compound of formula (Ia), as set forth above, is selected from the group consisting of:
1-(6,7-dihydro-5H-benzo[6,7]cyclohepta[1,2-c]pyridazin-3-yl)-N³-(4-((4-methylpiperazin-1-yl)methyl)phenyl)-1H-1,2,4-triazole-3,5-diamine;
1-(6,7-dihydro-5H-benzo[6,7]cyclohepta[1,2-c]pyridazin-3-yl)-N³-(4-((5-fluoroindolin-2-on-3-yl)methyl)phenyl)-1H-1,2,4-triazole-3,5-diamine;
1-(6,7-dihydro-5H-benzo[6,7]cyclohepta[1,2-c]pyridazin-3-yl)-N³-(4-((4-pyrrolidin-1-ylpiperidinyl)methyl)phenyl)-1H-1,2,4-triazole-3,5-diamine;
1-(6,7-dihydro-5H-benzo[6,7]cyclohepta[1,2-c]pyridazin-3-yl)-N³-(4-((4-cyclopentylpiperazinyl)methyl)phenyl)-1H-1,2,4-triazole-3,5-diamine;
1-(6,7-dihydro-5H-benzo[6,7]cyclohepta[1,2-c]pyridazin-3-yl)-N³-(4-((4-isopropylpiperazinyl)methyl)phenyl)-1H-1,2,4-triazole-3,5-diamine; and
1-(6,7-dihydro-5H-benzo[2,3]thiepino[4,5-c]pyridazin-3-yl)-N3-(3-fluoro-4-(isoindolin-2-yl)phenyl)-1H-1,2,4-triazole-3,5-diamine.

In some embodiments in the compound of formula (Ia) as set forth above:
R¹, R⁴ and R⁵ are each independently hydrogen;
R² is 6,7,8,9-tetrahydro-5H-benzo[7]annulene-2-yl optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted

heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyridimin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); and each R⁶, each R⁷, each R⁸, each R⁹, each R¹², each R¹³ and each R¹⁴ are as described above for compounds of formula (Ia).

In some embodiments the compound of formula (Ia), as set forth above, is selected from the group consisting of:
1-(6,7-dihydro-5H-benzo[6,7]cyclohepta[1,2-c]pyridazin-3-yl)-N³-(7-(pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((bicyclo[2.2. 1 ]heptan-2-yl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((bicyclo[2.2.1]heptan-2-yl)(methyl)amino) 6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-azetidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(R)-pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-diethylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-cyclopentylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(S)-pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(2-(S)-methyloxycarbonyl)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(2-(S)-carboxy)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(8-diethylaminoethyl-9hydroxy-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(3-(S)-fluoropyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(2-(S)-methylpyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(3-(R)-hydroxypyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(2-(R)-methylpyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(3-(S)-hydroxypyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(3-(R)-fluoropyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-oxo-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-cyclohexylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-cyclopropylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-hydroxy-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-methylpiperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(tetrahydrofuran-2-ylmethyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-cyclobutylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(cyclopropylmethyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(2-(diethylamino)ethyl)methylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-pyrrolidin-1-ylpiperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(piperidin-1-ylmethyl)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(2-(dimethylamino)ethyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(carboxymethyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(acetamido)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-((2R)-2-(methoxycarbonyl)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4,4-difluoropiperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((methoxycarbonylmethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((2R)-2-(carboxy)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(ethoxycarbonyl)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(carboxy)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((carboxymethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(ethoxycarbonylmethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(carboxymethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N³-((7S)-7-amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7s)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2-methylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((propyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(dipropylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(diethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(cyclopentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((1-cyclopentylethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(2-propylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((3,3-dimethylbut-2-yl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((5-chlorothien-2-yl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2-carboxyphenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((3-bromophenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(dimethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(3-pentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2,2-dimethylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(di(cyclopentylmethyl)amino)-6,7,8,9-tetrahydro-5H-benzo[7]annulene-2-yl)-1H-1,2,4-triazole-3,5-diamine;
((cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(di(bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(3-methylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(2-ethylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(but-2-enylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(butyl(but-2-enyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N⁵-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-amino-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(dimethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(diethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(dipropylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-((methylethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(cyclopentylamino)-6,7,8,9-tetrahydro-5H-benzo[7]annulene-2-yl)-1H-1,2,4-triazole-3,5-diamine; and
1-(6,7-dihydro-5H-pyrido[2′,3′:6,7]cyclohepta[1,2-c]pyridazin-3-yl)-N³-((7S)-7-(2-butylamino)-6,7,8,9-tetrahydro-5H-benzo[7]annulene-2-yl)-1H-1,2,4-triazole-3,5-diamine.

In some embodiments in the compound of formula (Ia) as set forth above:
R¹, R⁴ and R⁵ are each independently hydrogen;
R² is heteroaryl optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally

substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2);
R³ is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl,

optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); and each R⁶, each R⁷, each R⁸, each R⁹, each R¹², each R¹³ and each R¹⁴ are as described above for compounds of formula (Ia); and each R⁶, each R⁷, each R⁸, each R⁹, each R¹⁰, each R¹¹, each R¹², each R¹³ and each R¹⁴ are as described above for compounds of formula (Ia).

In some embodiments in the compound of formula (Ia) as set forth above:
R² is heteroaryl selected from the group consisting of pyridinyl, pyrimidinyl,

4,5-dihydro-1H-benzo[b]azepin-2(3H)-on-8-yl, benzo[d]imidazolyl,

6,7,8,9-tetrahydro-5H-pyrido[3,2-d]azepin-3-yl, 6,7,8,9-tetrahydro-5H-pyrido[3,2-c]azepin-3-yl,

5,6,7,8-tetrahydro-1,6-naphthyridin-3-yl, 5,6,7,8-tetrahydroquinolin-3-yl,

1,2,3,4-tetrahydroisoquinolin-7-yl, 2,3,4,5-tetrahydrobenzo[b]oxepin-7-yl,

3,4-dihydro-2H-benzo[b][1,4]dioxepin-7-yl, benzo[d]oxazol-5-yl, 3,4-dihydro-2H-benzo[b][1,4]oxazin-7-yl, benzo[b]thiophenyl, and

6,7,8,9-tetrahydro-5H-cyclohepta[b]pyridin-3-yl, each optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl,

optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)-R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,t][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl,

optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

Another embodiment is the use where, in the compound of formula (Ia) as set forth above: R² is selected from the group consisting of pyridinyl and pyrimidinyl, each optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally

substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)N(R¹²)₂ (where t is 1 or 2).

In some embodiments the compound of formula (Ia), as set forth above, is selected from the group consisting of:
1-(6,7-dihydro-5H-benzo[6,7]cyclohepta[1,2-c]pyridazin-3-yl)-N³-(6-(4-(bicyclo[2.2.1]heptan-2-yl)piperazin-1-yl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(4-cyclopentyl-1,4-diazepan-1-yl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(4-methylpiperazin-1-yl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(4-(4-methylpiperazin-1-yl)piperidin-1-yl)pyridine-3-yl)-1H-1,2,4-triazole-3,5-diamine;
1-(6,7-dihydro-5H-benzo[6,7]cyclohepta[1,2-c]pyridazin-3-yl)-N³-(pyridin-3-yl)-1H-1,2,4-triazole-3,5-diamine;
1-(6,7-dihydro-5H-benzo[6,7]cyclohepta[1,2-c]pyridazin-3-yl)-N³-(6-(6-aminopyridin-3-yl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(3-aminophenyl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(3-cyanophenyl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(benzo[d][1,3]dioxole-6-yl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(3-methylsulfonamidylphenyl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(2-diethylaminomethyl)pyrrolidin-1-ylpyridin-3-yl) -1H-1,2,4-triazole-3,5-diamine;
1-(6,7-dihydro-5H-benzo[6,7]cyclohepta[1,2-c]pyridazin-3-yl)-N³-(6-(3-diethylaminopyrrolidin-1-yl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(3-(4-(N-methylpiperazin-4-yl)piperidin-1-yl)-(E)-propenyl)pyridin-3-yl)-1H-1,2,4-triazole-3,5-diamine;
1-(6,7-dihydro-5H-benzo[6,7]cyclohepta[1,2-c]pyridazin-3-yl)-N3-(6-(4-(pyrrolidin-1-yl)piperidin-1-yl)-5-methylpyridin-3-yl)-1H-1,2,4-triazole-3,5-diamine;
1-(6,7-dihydro-5H-benzo[6,7]cyclohepta[1,2-c]pyridazin-3-yl)-N³-(6-(3-piperidin-1-yl-(E)-propenyl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(4-(bicyclo[2.2.1]heptan-2-yl)-1,4-diazepan-1-yl)pyridin-3-yl)-1H-1,2,4-triazole-3,5-diamine;
1-(6,7-dihydro-5H-benzo[6,7]cyclohepta[1,2-c]pyridazin-3-yl)-N3-(6-(3-(4-(pyrrolidin-1-yl)piperidin-1-yl)-(E)-propenyl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(3-piperidin-1-yl)-propanylpyridin-3-yl)-1H-1,2,4-triazole-3,5-diamine;
1-(6,7-dihydro-5H-benzo[6,7]cyclohepta[1,2-c]pyridazin-3-yl)-N³-(6-(3-(4-(piperidin-1-yl)piperidin-1-yl)-(E)-propenyl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(3-(4-dimethylaminopiperidin-1-yl)-(E)-propenyl)pyridin-3-yl)-1H-1,2,4-triazole-3,5-diamine;
1-(6,7-dihydro-5H-benzo[6,7]cyclohepta[1,2-c]pyridazin-3-yl)-N³-(2-(4-pyrrolidin-1-ylpiperidin-1-yl)pyrimidin-5-yl)-1H-1,2,4-triazole-3,5-diamine;
1-(6,7-dihydro-5H-benzo[6,7]cyclohepta[1,2-c]pyridazin-3-yl)-N³-(2-(4-(piperidin-1-ylmethyl)piperidin-1-yl)pyrimidin-5-yl)-1H-1,2,4-triazole-3,5-diamine;
1-(6,7-dihydro-5H-benzo[6,7]cyclohepta[1,2-c]pyridazin-3-yl)-N³-(6-((4-piperidin-1-ylpiperidin-1-yl)carbonyl)pyridin-3-yl)-1H-1,2,4-triazole-3,5-diamine;
1-(6,7-dihydro-5H-benzo[6,7]cyclohepta[1,2-c]pyridazin-3-yl)-N³-(2-(4-cyclopropylmethylpiperazin-1-yl)pyridine-5-yl)-1H-1,2,4-triazole-3,5-diamine; and
1-(6,7-dihydro-5H-benzo[6,7]cyclohepta[1,2-c]pyridazin-3-yl)-N³-(2-(3-(S)-methyl-4-cyclopropylmethylpiperazin-1-yl)pyridine-5-yl)-1H-1,2,4-triazole-3,5-diamine.

In some embodiments in the compound of formula (Ia) as set forth above:
R¹, R⁴ and R⁵ are each independently hydrogen;
R² is selected from the group consisting of 4,5-dihydro-1H-benzo[b]azepin-2(3H)-on-8-yl, benzo[d]imidazolyl, 6,7,8,9-tetrahydro-5H-pyrido[3,2-d]azepin-3-yl,

6,7,8,9-tetrahydro-5H-pyrido[3,2-c]azepin-3-yl, 5,6,7,8-tetrahydro-1,6-naphthyridin-3-yl,

5,6,7,8-tetrahydroquinolin-3-yl, 1,2,3,4-tetrahydroisoquinolin-7-yl,

2,3,4,5-tetrahydrobenzo[b]oxepin-7-yl, 3,4-dihydro-2H-benzo[b][1,4]dioxepin-7-yl,

benzo[d]oxazol-5-yl, 3,4-dihydro-2H-benzo[b][1,4]oxazin-7-yl, benzo[b]thiophenyl, and

6,7,8,9-tetrahydro-5H-cyclohepta[b]pyridin-3-yl, each optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally

substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,t][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl,

optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments the compound of formula (Ia), as set forth above, is selected from the group consisting of:
1-(6,7-dihydro-5H-benzo[6,7]cyclohepta[1,2-c]pyridazin-3-yl)-N³-(4,5-dihydro-1H-benzo[b]azepin-2(3H)-on-8-yl)-1H-1,2,4-triazole-3,5-diamine;
1-(6,7-dihydro-5H-benzo[6,7]cyclohepta[1,2-c]pyridazin-3-yl)-N³-(2-(dimethylaminomethyl)-1H-benzo[d]imidazol-5-yl)-1H-1,2,4-triazole-3,5-diamine;
1-(6,7-dihydro-5H-benzo[6,7]cyclohepta[1,2-c]pyridazin-3-yl)-N³-(7-cyclopentyl-6,7,8,9-tetrahydro-5H-pyrido[3,2-d]azepin-3-yl)-1H-1,2,4-triazole-3,5-diamine;
1-(6,7-dihydro-5H-benzo[6,7]cyclohepta[1,2-c]pyridazin-3-yl)-N³-(6-methyl-5,6,7,8-tetrahydro-1,6-naphthyridin-3-yl)-1H-1,2,4-triazole-3,5-diamine;
1-(6,7-dihydro-5H-benzo[6,7]cyclohepta[1,2-c]pyridazin-3-yl)-N³-(6-(4-(4-methylpiperazin-1-yl)piperidin-1-yl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(4-methylpiperazin-1-yl)carbonyl-5,6,7,8-tetrahydroquinolin-3-yl)-1H-1,2,4-triazole-3,5-diamine, compound #31, 1H-1,2,4-triazole-3,5-diamine;
1-(6,7-dihydro-5H-benzo[6,7]cyclohepta[1,2-c]pyridazin-3-yl)-N³-(2,3,4,5-tetrahydrobenzo[b]oxepin-7-yl)-1H-1,2,4-triazole-3,5-diamine;
1-(6,7-dihydro-5H-benzo[6,7]cyclohepta[1,2-c]pyridazin-3-yl)-N³-(3,4-dihydro-2H-benzo[b][1,4]dioxepin-7-yl)-1H-1,2,4-triazole-3,5-diamine;
1-(6,7-dihydro-5H-benzo[2,3]oxepino[4,5-c]pyridazin-3-yl)-N3-(2-(pyrrolidin-1-ylmethyl)benzo[d]oxazol-5-yl)-1H-1,2,4-triazole-3,5-diamine;

1-(6,7-dihydro-5H-benzo[6,7]cyclohepta[1,2-c]pyridazin-3-yl)-N³-(4-(2-dimethylaminoethyl)-(3,4-dihydro-2H-benzo[b][1,4]oxazin-7-yl))-1H-1,2,4-triazole-3,5-diamine;
1-(6,7-dihydro-5H-benzo[2,3]oxepino[4,5-c]pyridazin-3-yl)-N³-(4-(2-dimethylaminoethyl)-(3,4-dihydro-2H-benzo[b][1,4]oxazin-7-yl))-1H-1,2,4-triazole-3,5-diamine;
1-(6,7-dihydro-5H-benzo[2,3]thiepino[4,5-c]pyridazin-3-yl)-N³-(2-(1-(4-(2-(dimethylamino)ethyl)piperazin-1-yl)oxomethyl)benzo[b]thiophen-5-yl)-1H-1,2,4-triazole-3,5-diamine;
1-(6,7-dihydro-5H-benzo[6,7]cyclohepta[1,2-c]pyridazin-3-yl)-N³-(6-cyclopentyl-6,7,8,9-tetrahydro-5H-pyrido[3,2-c]azepin-3-yl) -1H-1,2,4-triazole-3,5-diamine;
1-(6,7-dihydro-5H-benzo[6,7]cyclohepta[1,2-c]pyridazin-3-yl)-N³-((7-pyrrolidin-1-yl)-6,7,8,9-tetrahydro-5H-cyclohepta[b]pyridine-3-yl)-1H-1,2,4-triazole-3,5-diamine;
1-(6,7-dihydro-5H-benzo[6,7]cyclohepta[1,2-c]pyridazin-3-yl)-N³ (2-cyclopentyl-1,2,3,4-tetrahydroisoquinolin-7-yl)-1H-1,2,4-triazole-3,5-diamine;
1-(6,7-dihydro-5H-benzo[6,7]cyclohepta[1,2-c]pyridazin-3-yl)-N³-(6-(pyrrolidin-1-yl)-5,6,7,8-tetrahydroquinolin-3-yl)-1H-1,2,4-triazole-3,5-diamine;
1-(6,7-dihydro-5H-benzo[6,7]cyclohepta[1,2-c]pyridazin-3-yl)-N³-(6-cyclopentyl-5,6,7,8-tetrahydro-1,6-naphthyridine-3-yl)-1H-1,2,4-triazole-3,5-diamine;
1-(6,7-dihydro-5H-benzo[6,7]cyclohepta[1,2-c]pyridazin-3-yl)-N³-((S)-7-(pyrrolidin-1-yl)-6,7,8,9-tetrahydro-5H-cyclohepta[b]pyridine-3-yl)-1H-1,2,4-triazole-3,5-diamine;
1-(6,7-dihydro-5H-benzo[6,7]cyclohepta[1,2-c]pyridazin-3-yl)-N³-(1,2,3,4-tetrahydroisoquinolin-7-yl)-1H-1,2,4-triazole-3,5-diamine;
1-(6,7-dihydro-5H-benzo[6,7]cyclohepta[1,2-c]pyridazin-3-yl)-N³-(2-(1-methylpiperidin-4-yl)-1,2,3,4-tetrahydroisoquinolin-7-yl)-1H-1,2,4-triazole-3,5-diamine; and
1-(6,7-dihydro-5H-benzo[6,7]cyclohepta[1,2-c]pyridazin-3-yl)-N³-(2-(cyclopropylmethyl)-1,2,3,4-tetrahydroisoquinolin-7-yl)-1H-1,2,4-triazole-3,5-diamine.

In some embodiments the compound of formula (Ia), as set forth above, is a compound of formula (Ia1):

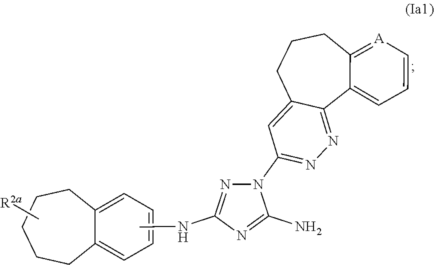

wherein:
A is ═C(H)— or ═N—;
each R^{2a} is independently selected from the group consisting of —N(R^{12a})₂ and —N(R^{12a})C(O)R^{12a},
or R^{2a} is an N-heterocyclyl optionally substituted by one or more substituents selected from the group consisting of halo and —R²¹—C(O)OR²⁰,
each R^{12a} is independently selected from the group consisting of hydrogen, alkyl, alkenyl, optionally substituted aralkyl, optionally substituted cycloalkyl, optionally substituted cycloalkylalkyl, optionally substituted heteroaryl and optionally substituted heteroarylalkyl; R²⁰ is independently selected from the group consisting of hydrogen, alkyl, alkenyl, optionally substituted aralkyl, optionally substituted cycloalkyl, optionally substituted cycloalkylalkyl, optionally substituted heteroaryl and optionally substituted heteroarylalkyl; and R²¹ is independently selected from the group consisting of a direct bond or an optionally substituted straight or branched alkylene chain;
as an isolated stereoisomer or mixture thereof, or a pharmaceutically acceptable salt thereof.

In some embodiments the compound of formula (I) is a compound of formula (Ib):

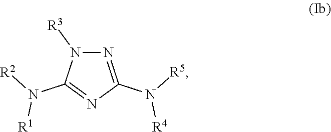

wherein R¹, R², R³, R⁴ and R⁵ are as described above for compounds of formula (I), as an isolated stereoisomer or mixture thereof or as a tautomer or mixture thereof, or a pharmaceutically acceptable salt or N-oxide thereof.

In some embodiments in the compound of formula (Ib) as set forth above, R² and R³ are each independently a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl,

optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); and R¹, R⁴, R⁵, each R⁶, each R⁷, each R⁸, each R⁹, each R¹⁰, each R¹¹ and R¹² are as described above in relation to formula (I).

In some embodiments in the compound of formula (Ib) as set forth above:
R¹, R⁴ and R⁵ are each hydrogen;
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
each R⁹ is independently selected from the group consisting of a direct bond and an optionally substituted straight or branched alkylene chain;
each R¹⁰ is an optionally substituted straight or branched alkylene chain; and each R¹¹ is independently selected from the group consisting of hydrogen, alkyl, cyano, nitro and —OR⁸.

In some embodiments in the compound of formula (Ib) as set forth above:
R² and R³ are each independently a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl,

optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments the compound of formula (Ib), as set forth above, is 1-(6,7-dihydro-5H-benzo[6,7]cyclohepta[1,2-c]pyridazin-3-yl)-N⁵-(5,7,8,9-tetrahydrospiro[cyclohepta[b]pyridine-6,2′[1,3]dioxolane]-3-yl)-1H-1,2,4-triazole-3,5-diamine.

In some embodiments in the compound of formula (Ib) as set forth above:
R² is selected from the group consisting of aryl and heteroaryl, where the aryl and the heteroaryl are each independently optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally

substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²), —R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl,

optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments in the compound of formula (Ib) as set forth above:
R¹, R⁴ and R⁵ are each independently hydrogen;
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, alkenyl, alkynyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
each R⁹ is independently selected from the group consisting of a direct bond and an optionally substituted straight or branched alkylene chain;
each R¹⁰ is an optionally substituted straight or branched alkylene chain;
each R¹¹ is independently selected from the group consisting of hydrogen, alkyl, cyano, nitro and —OR⁸;
each R¹² is independently selected from the group consisting of hydrogen, alkyl, alkenyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{12's}, together with the common nitrogen to which they are both attached, form an optionally substituted N-heterocyclyl or an optionally substituted N-heteroaryl;
each R¹³ is independently selected from the group consisting of a direct bond and an optionally substituted straight or branched alkylene chain; and
each R¹⁴ is an optionally substituted straight or branched alkylene chain.

In some embodiments in the compound of formula (Ib) as set forth above:
R² is aryl optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally

substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)2 (where t is 1 or 2); and
R³ is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl,

optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰-CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments in the compound of formula (Ib) as set forth above:
R¹, R⁴ and R⁵ are each independently hydrogen;
R² is aryl selected from the group consisting of phenyl and 6,7,8,9-tetrahydro-5H-benzo[7]annulene-2-yl, each optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally

substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)OR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,t][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl,

optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments in the compound of formula (Ib) as set forth above:
R² is phenyl optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally

substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2).

In some embodiments the compound of formula (Ib), as set forth above, is selected from the group consisting of:
1-(6,7-dihydro-5H-benzo[6,7]cyclohepta[1,2-c]pyridazin-3-yl)-N⁵-(3-fluoro-4-(4-(indolin-2-on-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-4-(4-(morpholin-4-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N⁵-(4-(3,5-dimethylpiperazin-1-yl)phenyl)-1H-1,2,4-triazole-3,5-diamine;
1-(7-methyl-6,7-dihydro-5H-benzo[2,3]azepino[4,5-c]pyridazin-3-yl)-N⁵-(4-(N-methylpiperazin-1-yl)phenyl)-1H-1,2,4-triazole-3,5-diamine;
1-(6,7-dihydro-5H-benzo[6,7]cyclohepta[1,2-c]pyridazin-3-yl)-N⁵-(4-((5-fluoroindolin-2-on-3-yl)methyl)phenyl)-1H-1,2,4-triazole-3,5-diamine;
1-(6,7-dihydro-5H-benzo[6,7]cyclohepta[1,2-c]pyridazin-3-yl)-N⁵-(4-(4-pyrrolidin-1-ylpiperidinyl)phenyl)-1H-1,2,4-triazole-3,5-diamine;
1-(6,7-dihydro-5H-benzo[6,7]cyclohepta[1,2-c]pyridazin-3-yl)-N⁵-(4-((4-pyrrolidin-1-ylpiperidinyl)methyl)phenyl)-1H-1,2,4-triazole-3,5-diamine;
1-(6,7-dihydro-5H-benzo[6,7]cyclohepta[1,2-c]pyridazin-3-yl)-N⁵-(4-((4-cyclopentylpiperazinyl)methyl)phenyl)-1H-1,2,4-triazole-3,5-diamine;
1-(6,7-dihydro-5H-benzo[6,7]cyclohepta[1,2-c]pyridazin-3-yl)-N⁵-(4-((4-isopropylpiperazinyl)methyl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-4-(4-N-methylpiperid-4-ylpiperazinyl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-4-(7-methyl-2,7-diazaspiro[4.4]nonan-2-yl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-4-(3-pyrrolidin-1-ylazetidinyl)phenyl)-1H-1,2,4-triazole-3,5-diamine;
1-(6,7-dihydro-5H-benzo[6,7]cyclohepta[1,2-c]pyridazin-3-yl)-N⁵-(3-methyl-4-(4-(N-methylpiperazin-4-yl)piperidin-1-yl)phenyl)-1H-1,2,4-triazole-3,5-diamine;
1-(6,7-dihydro-5H-benzo[2,3]thiepino[4,5-c]pyridazin-3-yl)-N5-(4-(4-pyrrolidin-1-ylpiperidinyl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-(4-(3-pyrrolidin-1-yl)pyrrolidin-1-yl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-4-(4-methylpiperazin-1-yl)phenyl)-1H-1,2,4-triazole-3,5-diamine; and
1-(6,7-dihydro-5H-benzo[6,7]cyclohepta[1,2-c]pyridazin-3-yl)-N⁵-(3-fluoro-4-(4-cyclopropylmethylpiperazin-1-yl)phenyl)-1H-1,2,4-triazole-3,5-diamine.

In some embodiments in the compound of formula (Ib) as set forth above:
R¹, R⁴ and R⁵ are each independently hydrogen;
R² is 6,7,8,9-tetrahydro-5H-benzo[7]annulene-2-yl optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted

heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³-S(O)ₜOR¹² (where t is 1 or 2), —R¹³-S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-cipyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1]-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl,

optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments the compound of formula (Ib), as set forth above, is selected from the group consisting of:
1-(6,7-dihydro-5H-benzo[6,7]cyclohepta[1,2-c]pyridazin-3-yl)-N⁵-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N⁵-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N⁵-(7-((bicyclo[2.2.1]heptan-2-yl)(methyl)amino)-6,7,8,9-tetrahydro-5H-benzo[7]annulene-2-yl)-1H-1,2,4-triazole-3,5-diamine; and
1-(6,7-dihydro-5H-benzo[6,7]cyclohepta[1,2-c]pyridazin-3-yl)-N⁵-(7-(S)-pyrrolidin-1-yl-6,7,8,9-tetrahydro-5H-benzo[7]annulene-2-yl)-1H-1,2,4-triazole-3,5-diamine.

In some embodiments in the compound of formula (Ib) as set forth above:
R¹, R⁴ and R⁵ are each independently hydrogen;
R² is heteroaryl optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally

substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O))N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)2 (where t is 1 or 2); and
R³ is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted

heterocyclyl, —R⁹—OR⁸, —R⁹—O—R¹⁰—OR⁸, —R⁹—O—R¹⁰—O—R¹⁰—OR⁸, —R⁹—O—R¹⁰-CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N (R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments in the compound of formula (Ib) as set forth above:
R² is heteroaryl selected from the group consisting of pyridinyl, pyrimidinyl,

4,5-dihydro-1H-benzo[b]azepin-2(3H)-on-8-yl, benzo[d]imidazolyl,

6,7,8,9-tetrahydro-5H-pyrido[3,2-d]azepin-3-yl, 6,7,8,9-tetrahydro-5H-pyrido[3,2-c]azepin-3-yl,

5,6,7,8-tetrahydro-1,6-naphthyridin-3-yl, 5,6,7,8-tetrahydroquinolin-3-yl,

1,2,3,4-tetrahydroisoquinolin-7-yl, 2,3,4,5-tetrahydrobenzo[b]oxepin-7-yl,

3,4-dihydro-2H-benzo[b][1,4]dioxepin-7-yl, benzo[d]oxazol-5-yl, 3,4-dihydro-2H-benzo[b][1,4]oxazin-7-yl, benzo[b]thiophenyl, and

6,7,8,9-tetrahydro-5H-cyclohepta[b]pyridin-3-yl, each optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl,

optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl,

optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments the compound of formula (Ib), as set forth above, is selected from the group consisting of:
1-(6,7-dihydro-5H-benzo[6,7]cyclohepta[1,2-c]pyridazin-3-yl)-N5-(6-(4-(pyrrolidin-1-yl)piperidin-1-yl)-5-methylpyridin-3-yl)-1H-1,2,4-triazole-3,5-diamine;
1-(6,7-dihydro-5H-benzo[6,7]cyclohepta[1,2-c]pyridazin-3-yl)-N⁵-(4-(3,5-dimethylpiperazin-1-yl)phenyl)-1H-1,2,4-triazole-3,5-diamine;
1-(6,7-dihydro-5H-benzo[6,7]cyclohepta[1,2-c]pyridazin-3-yl)-N⁵-(1,2,3,4-tetrahydroisoquinolin-7-yl)-1H-1,2,4-triazole-3,5-diamine; and
1-(6,7-dihydro-5H-benzo[6,7]cyclohepta[1,2-c]pyridazin-3-yl)-N⁵-(2-(1-methylpiperidin-4-yl)-1,2,3,4-tetrahydroisoquinolin-7-yl)-1H-1,2,4-triazole-3,5-diamine.

In some embodiments the compound of formula (Ib), as set forth above, is a compound of formula (1b1):

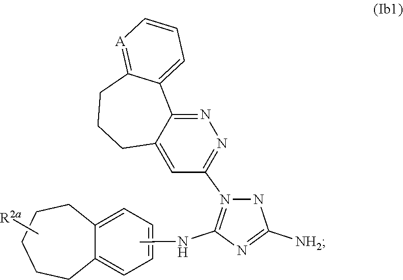

wherein:
A is ═C(H)— or ═N—;

each R^{2a} is independently selected from the group consisting of —N(R^{12a})₂ and —N(R^{12a})C(O)R^{12a},
or R^{2a} is an N-heterocyclyl optionally substituted by one or more substituents selected from the group consisting of halo and —R²¹—C(O)OR²⁰,
each R^{12a} is independently selected from the group consisting of hydrogen, alkyl, alkenyl, optionally substituted aralkyl, optionally substituted cycloalkyl, optionally substituted cycloalkylalkyl, optionally substituted heteroaryl and optionally substituted heteroarylalkyl;
R²⁰ is independently selected from the group consisting of hydrogen, alkyl, alkenyl, optionally substituted aralkyl, optionally substituted cycloalkyl, optionally substituted cycloalkylalkyl, optionally substituted heteroaryl and optionally substituted heteroarylalkyl; and
R²¹ is independently selected from the group consisting of a direct bond or an optionally substituted straight or branched alkylene chain;
as an isolated stereoisomer or mixture thereof, or a pharmaceutically acceptable salt thereof.

### Preferred Embodiments

Preferably, the AXL inhibitor is 1-(6,7-dihydro-5H-benzo[6,7]cyclohepta[1,2-c]pyridazin-3-yl)-N³-((7-(S)-pyrrolidin-1-yl)-6,7,8,9-tetrahydro-5H-benzo[7]annulene-2-yl)-1H-1,2,4-triazole-3,5-diamine.

The most preferred AXL inhibitor is Bemcentinib (CAS No. 1037624-75-1; UNII 0ICW2LX8AS). Bemcentinib may be referred to as BGB324 or R428.

### Other Embodiments

In some other embodiments the AXL inhibitor is selected from the group consisting of:
Dubermatinib (CAS No. 1341200-45-0; UNII 14D65TV20J);
Gilteritinib (CAS No. 1254053-43-4; UNII 66D92MGC8M);
Cabozantinib (CAS No. 849217-68-1; UNII 1C39JW444G);
SG17079 (CAS No. 1239875-86-5);
Merestinib (CAS No. 1206799-15-6; UNII 5OGS5K699E);
Amuvatinib (CAS No. 850879-09-3; UNII SO9S6QZB4R);
Bosutinib (CAS No. 380843-75-4 ; UNII 5018V4AEZ0);
Glesatinib (CAS No. 936694-12-1; UNII 7Q29OXD98N); and
foretinib (CAS No. 849217-64-7; UNII 81 FH7VK1C4).
TP0903 (CAS No. 1341200-45-0).

In some other embodiments the AXL inhibitor is an AXL inhibitor as described in any of the following references: WO2008/083367, WO2010/083465, and WO2012/028332 (the contents of each of which is hereby incorporated by reference).

Definitions

As used herein, unless specified to the contrary, the following terms have the meaning indicated:
“Amino” refers to the —NH₂ radical.
“Carboxy” refers to the —C(O)OH radical.
“Cyano” refers to the —CN radical.
“Nitro” refers to the —NO₂ radical.
“Oxa” refers to the —O— radical.
“Oxo” refers to the ═O radical.
“Thioxo” refers to the ═S radical.
“Alkyl” refers to a straight or branched hydrocarbon chain radical consisting solely of carbon and hydrogen atoms, containing no unsaturation, having from one to twelve carbon atoms, preferably one to eight carbon atoms or one to six carbon atoms and which is attached to the rest of the molecule by a single bond, for example, methyl, ethyl, n-propyl, 1-methylethyl (iso-propyl), n-butyl, n-pentyl, 1,1-dimethylethyl (t-butyl), 3-methylhexyl, 2-methylhexyl, and the like. For purposes of this disclosure, the term “lower alkyl” refers to an alkyl radical having one to six carbon atoms.

“Optionally substituted alkyl” refers to an alkyl radical, as defined above, which is optionally substituted by one or more substituents selected from the group consisting of halo, cyano, nitro, oxo, thioxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²⁰, —N(R²⁰)C(O)R²⁰, —N(R²⁰)S(O)2R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl.
“Alkenyl” refers to a straight or branched hydrocarbon chain radical consisting solely of carbon and hydrogen atoms, containing at least one double bond, having from two to twelve carbon atoms, preferably one to eight carbon atoms and which is attached to the rest of the molecule by a single bond, for example, ethenyl, prop-1-enyl, but-1-enyl, pent-1-enyl, and penta-1,4-dienyl.
“Optionally substituted alkenyl” refers to an alkenyl radical, as defined above, which is optionally substituted by one or more substituents selected from the group consisting of halo, cyano, nitro, oxo,

thioxo, trimethylsilanyl, —OR²⁰, —OC(O)—R²⁰, —N(R²⁰)₂, —C(O)R²⁰, —C(O)OR²⁰, —C(O)N(R²⁰)^{2,} —N(R²⁰)C(O)OR²⁰, —N(R²⁰)C(O)R²⁰, —N(R²⁰)S(O)₂R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl.
“Alkynyl” refers to a straight or branched hydrocarbon chain radical consisting solely of carbon and hydrogen atoms, containing at least one triple bond, optionally containing at least one double bond, having from two to twelve carbon atoms, preferably one to eight carbon atoms and which is attached to the rest of the molecule by a single bond, for example, ethynyl, propynyl, butynyl, pentynyl, and hexynyl.
“Optionally substituted alkynyl” refers to an alkynyl radical, as defined above, which is optionally substituted by one or more substituents selected from the group consisting of halo, cyano, nitro, oxo,

thioxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²⁰, —N(R²⁰)C(O)R²⁰, —N(R²⁰)S(O)₂R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰′s, together with the common nitrogen to which they are both attached, form an optionally substituted N-heterocyclyl or an optionally substituted N-heteroaryl.
“Straight or branched alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, and n-butylene. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain.
“Optionally substituted straight or branched alkylene chain” refers to an alkylene chain, as defined above, which is optionally substituted by one or more substituents selected from the group

consisting of halo, cyano, nitro, aryl, cycloalkyl, heterocyclyl, heteroaryl, oxo, thioxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²⁰, —N(R²⁰)C(O)R²⁰, —N(R²⁰)S(O)₂R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰′s, together with the common nitrogen to which they are both attached, form an optionally substituted N-heterocyclyl or an optionally substituted N-heteroaryl.
“Straight or branched alkenylene chain” refers to a straight or branched divalent hydrocarbon chain linking the rest of the molecule to a radical group, consisting solely of carbon and hydrogen, containing at least one double bond and having from two to twelve carbon atoms, for example, ethenylene, propenylene, and n-butenylene. The alkenylene chain is attached to the rest of the molecule through a double bond or a single bond and to the radical group through a double bond or a single bond. The points of attachment of the alkenylene chain to the rest of the molecule and to the radical group can be through one carbon or any two carbons within the chain.
“Optionally substituted straight or branched alkenylene chain” refers to an alkenylene chain, as defined above, which is optionally substituted by one or more substituents selected from the group consisting of halo, cyano, nitro, aryl, cycloalkyl, heterocyclyl, heteroaryl, oxo, thioxo,

trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²⁰, —N(R²⁰)C(O)R²⁰, —N(R²⁰)S(O)₂R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl.
“Straight or branched alkynylene chain” refers to a straight or branched divalent hydrocarbon chain linking the rest of the molecule to a radical group, consisting solely of carbon and hydrogen, containing at least one triple bond and having from two to twelve carbon atoms, for example, propynylene, and n-butynylene. The alkynylene chain is attached to the rest of the molecule through a single bond and to the radical group through a double bond or a single bond. The points of attachment of the alkynylene chain to the rest of the molecule and to the radical group can be through one carbon or any two carbons within the chain.
“Optionally substituted straight or branched alkynylene chain” refers to an alkynylene chain, as defined above, which is optionally substituted by one or more substituents selected from the group consisting of alkyl, alkenyl, halo, haloalkenyl, cyano, nitro, aryl, cycloalkyl, heterocyclyl, heteroaryl, oxo,

thioxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²⁰, —N(R²⁰)C(O)R²⁰, —N(R²⁰)S(O)₂R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl.
“Aryl” refers to a hydrocarbon ring system radical comprising hydrogen, 6 to 14 carbon atoms and at least one aromatic ring. For purposes of this disclosure, the aryl radical may be a monocyclic, bicyclic, or tricyclic system and which may include spiro ring systems. An aryl radical is commonly, but not necessarily, attached to the parent molecule via an aromatic ring of the aryl radical. For purposes of this disclosure, an “aryl” radical as defined herein cannot contain rings having more than 7 members and cannot contain rings wherein two non-adjacent ring atoms thereof are connected through an atom or a group of atoms (i.e., a bridged ring system). Aryl radicals include, but are not limited to, aryl radicals derived from acenaphthylene, anthracene, azulene, benzene, 6,7,8,9-tetrahydro-5H-benzo[7]annulene, fluorene, as-indacene, s-indacene, indane, indene, naphthalene, phenalene, and phenanthrene.
“Optionally substituted aryl” refers to an aryl radical, as defined above, which is optionally substituted by one or more substituents selected from the group consisting of alkyl, alkenyl, alkynyl, halo, haloalkyl, haloalkenyl, haloalkynyl, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂—R²¹—O—R²²—C(O)N(R²⁰)₂, —R²¹—N(R²⁰)C(O)OR²⁰, —R²¹—N(R²⁰)C(O)R²⁰, —R²¹—N(R²⁰)S(O)₂R²⁰, —R²¹—C(═NR²⁰)N(R²⁰)₂—R²¹—S(O)ₜOR²⁰ (where t is 1 or 2), —R²¹—S(O)ₚR²⁰ (where p is 0, 1 or 2), and —R²¹—S(O)₂N(R²⁰)₂, where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl, each R²¹ is independently a direct bond or a straight or branched alkylene or alkenylene chain, and R²² is a straight or branched alkylene or alkenylene chain.
“Aralkyl” refers to a radical of the formula —R_{b}—R_{c} where R_{b} is an alkylene chain as defined above and R_{c} is one or more aryl radicals as defined above, for example, benzyl and diphenylmethyl.
“Optionally substituted aralkyl” refers to an aralkyl radical, as defined above, wherein the alkylene chain of the aralkyl radical is an optionally substituted alkylene chain, as defined above, and each aryl radical of the aralkyl radical is an optionally substituted aryl radical, as defined above.
“Aralkenyl” refers to a radical of the formula —R_{d}—R_{c} where R_{d} is an alkenylene chain as defined above and R_{c} is one or more aryl radicals as defined above.
“Optionally substituted aralkenyl” refers to an aralkenyl radical, as defined above, wherein the alkenylene chain of the aralkenyl radical is an optionally substituted alkenylene chain, as defined above, and each aryl radical of the aralkenyl radical is an optionally substituted aryl radical, as defined above.
“Aralkynyl” refers to a radical of the formula —RₑR_{c} where Rₑ is an alkynylene chain as defined above and R_{c} is one or more aryl radicals as defined above.
“Optionally substituted aralkynyl” refers to an aralkynyl radical, as defined above, wherein the alkynylene chain of the aralkynyl radical is an optionally substituted alkynylene chain, as defined above, and each aryl radical of the aralkynyl radical is an optionally substituted aryl radical, as defined above.
“Cycloalkyl” refers to a stable non-aromatic monocyclic or polycyclic hydrocarbon radical consisting solely of carbon and hydrogen atoms, which includes fused, spiro or bridged ring systems, having from three to fifteen carbon atoms, preferably having from three to ten carbon atoms, more preferably from five to seven carbons and which is saturated or unsaturated and attached to the rest of the molecule by a single bond. For purposes of this disclosure, a bridged ring system is a system wherein two non-adjacent ring atoms thereof are connected through an atom or a group of atoms, wherein the atom or the group of atoms are the bridging element. An example of a bridged cycloalkyl (monovalent) radical is norbornanyl (also called bicyclo[2.2.1]heptanyl). For purposes of this disclosure, a non-bridged ring system is a system which does not contain a bridging element, as described above. For purposes of this disclosure, a fused ring system is a system wherein two adjacent ring atoms thereof are connected through an atom or a group of atoms. An example of a fused cycloalkyl (monovalent) radical is decahydronaphthalenyl (also called decalinyl). For purposes of this disclosure, a spiro ring system is a system wherein two rings are joined via a single carbon (quaternary) atom. An example of a spiro cycloalkyl (monovalent) radical is spiro[5.5]undecanyl. Monocyclic cycloalkyl radicals do not include spiro, fused or bridged cycloalkyl radicals, but do include for example, cyclopropyl, cyclobutyl, cyclopentyl, cyclohexyl, cycloheptyl, and cyclooctyl. Polycyclic radicals include fused, spiro or bridged cycloalkyl radicals, for example, C₁₀ radicals such as adamantanyl (bridged) and decalinyl (fused), and C₇ radicals such as bicyclo[3.2.0]heptanyl (fused), norbornanyl and norbornenyl (bridged), as well as substituted polycyclic radicals, for example, substituted C₇ radicals such as 7,7-dimethylbicyclo[2.2.1]heptanyl (bridged).
“Optionally substituted cycloalkyl” refers to a cycloalkyl radical, as defined above, which is optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally

substituted heteroarylalkynyl, —R²¹—OR²⁰, —R²¹—OC(O)—R²⁰, —R²¹—N(R²⁰)₂, —R²¹—C(O)R²⁰, —R21—C(O)OR²⁰, —R²¹—C(O)N(R²⁰)₂, —R²¹—N(R²⁰)C(O)OR²⁰, —R²¹—N(R²⁰)C(O)R²⁰, —R²¹—N(R²⁰)S(O)₂R²⁰, —R²¹—C(═NR²⁰)N(R²⁰)₂, —R²¹—S(O)ₜOR²⁰ (where t is 1 or 2), —R²¹—S(O)ₚR²⁰ (where p is 0, 1 or 2), and —R²¹—S(O)₂N(R²⁰)₂, where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl, and each R²¹ is independently a direct bond or a straight or branched alkylene or alkenylene chain.
“Cycloalkylalkyl” refers to a radical of the formula —R_{b}R_{g} where R_{b} is an alkylene chain as defined above and R_{g} is a cycloalkyl radical as defined above.
“Optionally substituted cycloalkylalkyl” refers to a cycloalkylalkyl radical, as defined above, wherein the alkylene chain of the cycloalkylalkyl radical is an optionally substituted alkylene chain, as defined above, and the cycloalkyl radical of the cycloalkylalkyl radical is an optionally substituted cycloalkyl radical, as defined above.
“Cycloalkylalkenyl” refers to a radical of the formula —R_{d}R_{g} where R_{d} is an alkenylene chain as defined above and R_{g} is a cycloalkyl radical as defined above.
“Optionally substituted cycloalkylalkenyl” refers to a cycloalkylalkenyl radical, as defined above, wherein the alkenylene chain of the cycloalkylalkenyl radical is an optionally substituted alkenylene chain, as defined above, and the cycloalkyl radical of the cycloalkylalkenyl radical is an optionally substituted cycloalkyl radical as defined above.
“Cycloalkylalkynyl” refers to a radical of the formula —RₑR_{g} where Rₑ is an alkynylene radical as defined above and R_{g} is a cycloalkyl radical as defined above.
“Optionally substituted cycloalkylalkynyl” refers to a cycloalkylalkynyl radical, as defined above, wherein the alkynylene chain of the cycloalkylalkynyl radical is an optionally substituted alkynylene chain, as defined above, and the cycloalkyl radical of the cycloalkylalkynyl radical is an optionally substituted cycloalkyl radical as defined above.
“Halo” refers to bromo, chloro, fluoro or iodo.
“Haloalkyl” refers to an alkyl radical, as defined above, that is substituted by one or more halo radicals, as defined above, for example, trifluoromethyl, difluoromethyl, trichloromethyl, 2,2,2-trifluoroethyl, 1-fluoromethyl-2-fluoroethyl, 3-bromo-2-fluoropropyl, and 1-bromomethyl-2-bromoethyl.
“Haloalkenyl” refers to an alkenyl radical, as defined above that is substituted by one or more halo radicals, as defined above.
“Haloalkynyl” refers to an alkynyl radical, as defined above that is substituted by one or more halo radicals, as defined above.
“Heterocyclyl” refers to a stable 3- to 18-membered non-aromatic ring system radical which comprises one to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include spiro or bridged ring systems; and the nitrogen, carbon or sulfur atoms in the heterocyclyl radical may be optionally oxidized; the nitrogen atom may be optionally quaternized; and the heterocyclyl radical may be

partially or fully saturated. Examples of a bridged heterocyclyl include, but are not limited to, aza bicyclo[2.2.1]heptanyl, diazabicyclo[2.2.1]heptanyl, diazabicyclo[2.2.2]octanyl, diazabicyclo[3.2.1]octanyl, diazabicyclo[3.3.1]nonanyl, diazabicyclo[3.2.2]nonanyl and oxazabicyclo[2.2.1]heptanyl. A “bridged N-heterocyclyl” is a bridged heterocyclyl containing at least one nitrogen, but which optionally contains up to four additional heteroatoms selected from O, N and S. For purposes of this disclosure, a non-bridged ring system is a system wherein no two non-adjacent ring atoms thereof are connected through an atom or a group of atoms. Examples of heterocyclyl radicals include, but are not limited to, dioxolanyl, 1,4-diazepanyl, decahydroisoquinolyl, imidazolinyl, imidazolidinyl, isothiazolidinyl, isoxazolidinyl, morpholinyl, octahydroindolyl, octahydroisoindolyl, octahydro-1H-pyrrolo[3,2-c]pyridinyl, octahydro-1H-pyrrolo[2,3-c]pyridinyl, octahydro-1H-pyrrolo[2,3-b]pyridinyl, octahydro-1H-pyrrolo[3,4-b]pyridinyl, octahydropyrrolo[3,4-c]pyrrolyl, octahydro-1H-pyrido[1,2-a]pyrazinyl, 2-oxopiperazinyl, 2-oxopiperidinyl, 2-oxopyrrolidinyl, oxazolidinyl, 3,7-diazabicyclo[3.3.1]nonan-3-yl, piperidinyl, piperazinyl, 4-piperidonyl, pyrrolidinyl, pyrazolidinyl, quinuclidinyl, thiazolidinyl, tetrahydrofuranyl, thienyl[1,3]dithianyl, trithianyl, tetrahydropyranyl, thiomorpholinyl, thiamorpholinyl, 1-oxo-thiomorpholinyl, 1,1-dioxo-thiomorpholinyl, azetidinyl, octahydropyrrolo[3,4-c]pyrrolyl, octahydropyrrolo[3,4-b]pyrrolyl, decahydroprazino[1,2-a]azepinyl, azepanyl, azabicyclo[3.2.1]octyl, and 2,7-diazaspiro[4.4]nonanyl.
“Optionally substituted heterocyclyl” refers to a heterocyclyl radical, as defined above, which is optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted

heteroarylalkenyl, optionally substituted heteroarylalkynyl, —R²¹13 OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²⁰, —R²¹—N(R²⁰)C(O)R²⁰, —R²¹—N(R²⁰)S(O)₂R²⁰, —R²¹—C(═NR²⁰)N(R²⁰)₂, —R²¹13 S(O)ₜOR²⁰ (where t is 1 or 2), —R²¹—S(O)ₚR²⁰ (where p is 0, 1 or 2), and —R²¹—S(O)₂N(R²⁰)₂, where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl, and each R²¹ is independently a direct bond or a straight or branched alkylene or alkenylene chain.
“N-heterocyclyl” refers to a heterocyclyl radical as defined above containing at least one nitrogen and where the point of attachment of the N-heterocyclyl radical to the rest of the molecule may be through a nitrogen atom in the N-heterocyclyl radical or through a carbon in the N-heterocyclyl radical.
“Optionally substituted N-heterocyclyl” refers to an N-heterocyclyl, as defined above, which is optionally substituted by one or more substituents as defined above for optionally substituted heterocyclyl.
“Heterocyclylalkyl” refers to a radical of the formula —R_{b}Rₕ where R_{b} is an alkylene chain as defined above and Rₕ is a heterocyclyl radical as defined above, and when the heterocyclyl is a nitrogen-containing heterocyclyl, the heterocyclyl may be attached to the alkylene chain at the nitrogen atom.
“Optionally substituted heterocyclylalkyl” refers to a heterocyclylalkyl radical, as defined above, wherein the alkylene chain of the heterocyclylalkyl radical is an optionally substituted alkylene chain, as defined above, and the heterocyclyl radical of the heterocyclylalkyl radical is an optionally substituted heterocyclyl radical, as defined above.
“Heterocyclylalkenyl” refers to a radical of the formula —R_{d}Rₕ where R_{d} is an alkenylene chain as defined above and Rₕ is a heterocyclyl radical as defined above, and when the heterocyclyl is a nitrogen-containing heterocyclyl, the heterocyclyl may be attached to the alkenylene chain at the nitrogen atom.
“Optionally substituted heterocyclylalkenyl” refers to a heterocyclylalkenyl radical, as defined above, wherein the alkenylene chain of the heterocyclylalkenyl radical is an optionally substituted alkenylene chain, as defined above, and the heterocyclyl radical of the heterocyclylalkenyl radical is an optionally substituted heterocyclyl radical, as defined above.
“Heterocyclylalkynyl” refers to a radical of the formula —RₑRₕ where Rₑ is an alkynylene chain as defined above and Rₕ is a heterocyclyl radical as defined above, and when the heterocyclyl is a nitrogen-containing heterocyclyl, the heterocyclyl may be attached to the alkynylene chain at the nitrogen atom.
“Optionally substituted heterocyclylalkynyl” refers to a heterocyclylalkynyl radical, as defined above, wherein the alkynylene chain of the heterocyclylalkynyl radical is an optionally substituted alkynylene chain, as defined above, and the heterocyclyl radical of the heterocyclylalkynyl radical is an optionally substituted heterocyclyl radical, as defined above.
“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. A heteroaryl radical is commonly, but not necessarily, attached to the parent molecule via an aromatic ring of the heteroaryl radical. For purposes of this disclosure, the heteroaryl radical may be a monocyclic, bicyclic or tricyclic ring system, which may include spiro or bridged ring systems; and the nitrogen, carbon or sulfur atoms in the heteroaryl radical may be optionally oxidized and the nitrogen atom may be optionally quaternized. For purposes of this disclosure, the aromatic ring of the heteroaryl radical need not contain a heteroatom, as long as one ring of the heteroaryl radical contains a heteroatom. For example benzo-fused heterocyclyls such as 1,2,3,4-tetrahydroisoquinolin-7-yl are considered a “heteroaryl” for the purposes of this disclosure. Except for the polycyclic heteroaryls containing more than 14 ring atoms, as defined below, a “heteroaryl” radical as defined herein cannot contain rings having more than 7 members and cannot contain rings wherein two non-adjacent members thereof are connected through an atom or a group of atoms (i.e., a bridged ring system). Examples of heteroaryl radicals include, but are not limited to, azepinyl, acridinyl, benzimidazolyl, benzindolyl, 1,3-benzodioxolyl, benzofuranyl, benzoxazolyl, benzothiazolyl, benzothiadiazolyl, benzo[b][1,4]dioxepinyl, benzo[b][1,4]oxazinyl, benzo[b]azepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 3,4-dihydro-2H-benzo[b][1,4]dioxepinyl, cyclopenta[4,5]thieno[2,3-d]pyrimidinyl such as 6,7-dihydro-5H-cyclopenta[4,5]thieno[2,3-d]pyrimidinyl, 5,6-dihydrobenzo[h]quinazolinyl, 3,4-dihydro-2H-benzo[b][1,4]thiazinyl, 5,6-dihydrobenzo[h]cinnolinyl, 7′,8′-dihydro-5′H-spiro[[1,3]dioxolane-2,6′-quinoline]-3′-yl, 6,7-dihydro-5H-benzo[6,7]cyclohepta[1,2-c]pyridazinyl, 2,3-dihydro-1H-pyrido[2,3-b][1,4]oxazinyl, 3′,4′-dihydrospiro[cyclobutane-1,2′-pyrido[3,2-b][1,4]oxazinyl, dihydropyridooxazinyl such as 3,4-dihydro-2H-pyrido[3,2-b][1,4]oxazinyl, dihydropyridothiazinyl such as 3,4-dihydro-2H-pyrido[3,2-b][1,4]thiazinyl, dibenzofuranyl, dibenzothiophenyl, furanyl, furanonyl, furo[3,2-c]pyridinyl, furopyrimidinyl, furopyridazinyl, furopyrazinyl, isothiazolyl, imidazolyl, imidazopyrimidinyl, imidazopyridazinyl, imidazopyrazinyl, imidazo[1,2-a]pyridinyl, indazolyl, indolyl, indazolyl, isoindolyl, indolinyl, isoindolinyl, isoquinolinyl (isoquinolyl), indolizinyl, isoxazolyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 3′-oxo-3′,4′-dihydrospiro[cyclobutane-1,2′-pyrido[3,2-b][1,4]oxazine]yl, 7-oxo-5,6,7,8-tetrahydro-1,8-naphthyridinyl, 1-phenyl-1H-pyrrolyl, phenazinyl, phenothiazinyl, phenoxazinyl, phthalazinyl, phenanthridinyl, pteridinyl, purinyl, pyrrolyl, pyrazolyl, pyrazolo[3,4-d]pyrimidinyl, pyridinyl (pyridyl), pyrido[3,2-d]pyrimidinyl, pyrido[3,4-d]pyrimidinyl, pyrazinyl, pyrimidinyl, pyridazinyl (pyridazyl), pyrrolyl, pyrrolopyrimidinyl, pyrrolopyridazinyl, pyrrolopyrazinyl, 2H-pyrido[3,2-b][1,4]oxazinonyl, 1H-pyrido[2,3-b][1,4]oxazinonyl, pyrrolopyridinyl such as 1H-pyrrolo[2,3-b]pyridinyl, quinazolinyl, quinoxalinyl, quinolinyl, quinuclidinyl, tetrahydroquinolinyl, 5,6,7,8-tetrahydroquinazolinyl, 2,3,4,5-tetrahydrobenzo[b]oxepinyl, 6,7,8,9-tetrahydro-5H-cyclohepta[b]pyridinyl, 6,7,8,9-tetrahydro-5H-pyrido[3,2-c]azepinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, 1,2,3,4-tetrahydroisoquinolin-7-yl, triazinyl, thieno[2,3-d]pyrimidinyl, thienopyrimidinyl (e.g., thieno[3,2-d]pyrimidinyl), thieno[2,3-c]pyridinyl, thienopyridazinyl, thienopyrazinyl, and thiophenyl (thienyl).
“Optionally substituted heteroaryl” refers to a heteroaryl radical, as defined above, which is optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted

heteroarylalkenyl, optionally substituted heteroarylalkynyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²⁰, —R²¹—N(R²⁰)C(O)R²⁰, —R²¹—N(R²⁰)S(O)₂R²⁰2, —R²¹—C(═NR²⁰)N(R²⁰)₂, —R²¹—S(O)ₜOR²⁰ (where t is 1 or 2), —R²¹—S(O)ₚR²⁰ (where p is 0, 1 or 2), and —R²¹—S(O)₂N(R²⁰)₂, where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl, and each R²¹ is independently a direct bond or a straight or branched alkylene or alkenylene chain.
“N-heteroaryl” refers to a heteroaryl radical as defined above containing at least one nitrogen and where the point of attachment of the N-heteroaryl radical to the rest of the molecule may be through a nitrogen atom in the N-heteroaryl radical or through a carbon atom in the N-heteroaryl radical.
“Optionally substituted N-heteroaryl” refers to an N-heteroaryl, as defined above, which is optionally substituted by one or more substituents as defined above for optionally substituted heteroaryl.
“Polycyclic heteroaryl containing more than 14 ring atoms” refers to a 15- to 20-membered ring system radical comprising hydrogen atoms, one to fourteen carbon atoms, one to eight heteroatoms selected from the group consisting of nitrogen, oxygen and sulfur, and at least one aromatic ring. A “polycyclic heteroaryl containing more than 14 ring atoms” radical is commonly, but not necessarily, attached to the parent molecule via an aromatic ring of the “polycyclic heteroaryl containing more than 14 ring atoms” radical. For purposes of this disclosure, the “polycyclic heteroaryl containing more than 14 ring atoms” radical may be a bicyclic, tricyclic or tetracyclic ring system, which may include fused or spiro ring systems; and the nitrogen, carbon or sulfur atoms in the “polycyclic heteroaryl containing more than 14 ring atoms” radical may be optionally oxidized and the nitrogen atom may also be optionally quaternized. For purposes of this disclosure, the aromatic ring of the “polycyclic heteroaryl containing more than 14 ring atoms” radical need not contain a heteroatom, as long as one ring of the “polycyclic heteroaryl containing more than 14 ring atoms” radical contains a heteroatom. Examples of “polycyclic heteroaryl containing more than 14 ring atoms” radicals include, but are not limited to, 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl.
“Optionally substituted polycyclic heteroaryl containing more than 14 ring atoms” is meant to include “polycyclic heteroaryl containing more than 14 ring atoms” radicals, as defined above, which are optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted

heteroarylalkenyl, optionally substituted heteroarylalkynyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²⁰, —R²¹—N(R²⁰)C(O)R²⁰, —R²¹—N(R²⁰S(O)ₜR²⁰ (where t is 1 or 2), —R²¹—S(O)ₜOR²⁰ (where t is 1 or 2), —R²¹—S(O)ₚR²⁰ (where p is 0, 1 or 2), and —R²¹—S(O)ₜN(R²⁰)₂ (where t is 1 or 2), where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, may optionally form an optionally substituted N-heterocyclyl or an optionally substituted N-heteroaryl, and each R²¹ is independently a direct bond or a straight or branched alkylene or alkenylene chain.
“Heteroarylalkyl” refers to a radical of the formula —R_{b}Rᵢ where R_{b} is an alkylene chain as defined above and Rᵢ is a heteroaryl radical as defined above, and when the heteroaryl is a nitrogen-containing heteroaryl, the heteroaryl may be attached to the alkylene chain at the nitrogen atom.
“Optionally substituted heteroarylalkyl” refers to a heteroarylalkyl radical, as defined above, wherein the alkylene chain of the heteroarylalkyl radical is an optionally substituted alkylene chain, as defined above, and the heteroaryl radical of the heteroarylalkyl radical is an optionally substituted heteroaryl radical, as defined above.
“Heteroarylalkenyl” refers to a radical of the formula —R_{d}Rᵢ where R_{d} is an alkenylene chain as defined above and Rᵢ is a heteroaryl radical as defined above, and when the heteroaryl is a nitrogen-containing heteroaryl, the heteroaryl may be attached to the alkenylene chain at the nitrogen atom.
“Optionally substituted heteroarylalkenyl” refers to a heteroarylalkenyl radical, as defined above, wherein the alkenylene chain of the heteroarylalkenyl radical is an optionally substituted alkenylene chain, as defined above, and the heteroaryl radical of the heteroarylalkenyl radical is an optionally substituted heteroaryl radical, as defined above. “Heteroarylalkynyl” refers to a radical of the formula —RₑRᵢ where Rₑ is an alkynylene chain as defined above and Rᵢ is a heteroaryl radical as defined above, and when the heteroaryl is a nitrogen-containing heteroaryl, the heteroaryl may be attached to the alkynylene chain at the nitrogen atom.
“Optionally substituted heteroarylalkynyl” refers to a heteroarylalkynyl radical, as defined above, wherein the alkynylene chain of the heteroarylalkynyl radical is an optionally substituted alkynylene chain, as defined above, and the heteroaryl radical of the heteroarylalkynyl radical is an optionally substituted heteroaryl radical, as defined above.
“Hydroxyalkyl” refers to an alkyl radical as defined above which is substituted by one or more hydroxy radicals (—OH).

Certain chemical groups named herein may be preceded by a shorthand notation indicating the total number of carbon atoms that are to be found in the indicated chemical group. For example; C₇-C₁₂ alkyl

describes an alkyl group, as defined below, having a total of 7 to 12 carbon atoms, and C₄-C₁₂cy cloalkylalkyl describes a cycloalkylalkyl group, as defined below, having a total of 4 to 12 carbon atoms. The total number of carbons in the shorthand notation does not include carbons that may exist in substituents of the group described.

The compounds of formula (I), or their pharmaceutically acceptable salts, may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, such as HPLC using a chiral column. When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present disclosure includes tautomers of any said compounds.

“Atropisomers” are stereoisomers resulting from hindered rotation about single bonds where the barrier to rotation is high enough to allow for the isolation of the conformers (Eliel, E. L.; Wilen, S. H. Stereochemistry of Organic Compounds; Wiley & Sons: New York, 1994; Chapter 14). Atropisomerism is significant because it introduces an element of chirality in the absence of stereogenic atoms. The disclosure is meant to encompass atropisomers, for example in cases of limited rotation around the single bonds emanating from the core triazole structure, atropisomers are also possible and are also specifically included in the compounds of the disclosure.

The chemical naming protocol and structure diagrams used herein are a modified form of the IUPAC nomenclature system wherein the compounds of formula (I) are named herein as derivatives of the central core structure, i.e., the triazole structure. For complex chemical names employed herein, a substituent group is named before the group to which it attaches. For example, cyclopropylethyl comprises an ethyl backbone with cyclopropyl substituent. In chemical structure diagrams, all bonds are identified, except for some carbon atoms, which are assumed to be bonded to sufficient hydrogen atoms to complete the valency.

For purposes of this disclosure, the depiction of the bond attaching the R³ substituent to the parent triazole moiety in formula (I), as shown below:

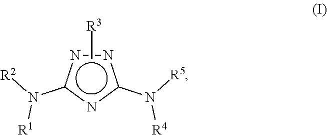

is intended to include only the two regioisomers shown below, i.e., compounds of formula (Ia) and (Ib):

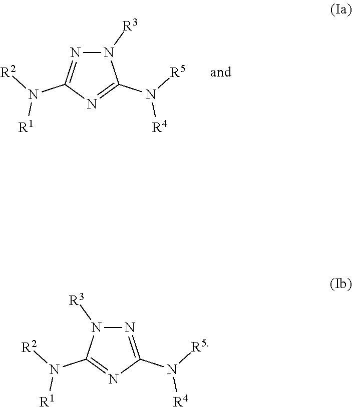

The numbering system of the ring atoms in compounds of formula (Ia) is shown below:

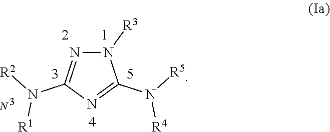

For example, a compound of formula (Ia) wherein R¹, R⁴ and R⁵ are each hydrogen, R² is 4-(2-(pyrrolidin-1-yl)ethoxy)phenyl and R³ is 6,7-dihydro-5H-benzo[6,7]cyclohepta[1,2-c]pyridazin-3-yl; i.e., a compound of the following formula:

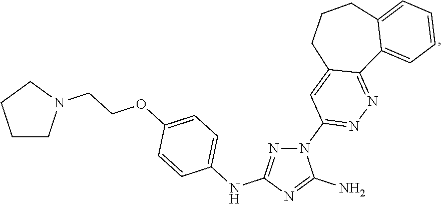 [SMILES: Nc1nc(Nc2ccc(OCCN3CCCC3)cc2)nn1-c1cc2c(nn1)-c1ccccc1CCC2]

is named herein as 1-(6,7-dihydro-5H-benzo[6,7]cyclohepta[1,2-c]pyridazin-3-yl)-N³-(4-(2-(pyrrolidin-1-yl)ethoxy)phenyl)-1H-1,2,4-triazole-3,5-diamine.

The numbering system of the ring atoms in compounds of formula (Ib) is shown below:

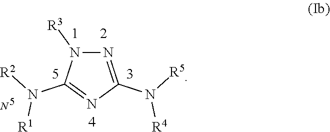

Compounds of formula (Ib) are similarly named herein.

Antibody AXL Inhibitors

In some embodiments the AXL inhibitor is an antibody. Preferably the antibody has AXL inhibitory activity. In some cases the antibody inhibits the binding of AXL to the GAS6 ligand.

In some embodiments, the anti-AXL antibody is an antibody as described in any of the following references: WO/2016/097370, WO/2017/220695, WO/2015/193428, WO/2016/166296, WO/2015/193430, EP2267454, WO/2009/063965, WO/2011/159980, WO/2012/175691, WO/2012/175692, WO/2013/064685, WO/2014/068139, WO/2009/062690, and WO/2010/130751 (the contents of each of which is hereby incorporated by reference).

In another embodiment, the anti-AXL antibody is an antibody as described in international patent application WO/2015/193428, the contents of which is hereby incorporated by reference, particularly as shown at pages 82-83.

In another embodiment, the anti-AXL antibody is an antibody as described in international patent application WO/2016/166296, the contents of which is hereby incorporated by reference, particularly the humanized 1H12 antibody disclosed therein.

In another embodiment, the anti-AXL antibody is an antibody as described in international patent application WO/2015/193430, the contents of which is hereby incorporated by reference, particularly as shown at pages 72-73.

In another embodiment, the anti-AXL antibody is an antibody as described in European patent publication EP2267454, the contents of which is hereby incorporated by reference.

In another embodiment, the anti-AXL antibody is an antibody as described in European patent publication WO/2009/063965, the contents of which is hereby incorporated by reference, particularly as shown at pages 31-33.

In another embodiment, the anti-AXL antibody is an antibody as described in US patent publication US 2012/0121587 A1, the contents of which is hereby incorporated by reference, particularly as shown at pages 26-61.

In another embodiment, the anti-AXL antibody is an antibody as described in international patent publication WO/2011/159980, the contents of which is hereby incorporated by reference, particularly the YW327.6S2 antibody as shown in FIG. 2, Figure page 6 (of 24).

In another embodiment, the anti-AXL antibody is an antibody as described in international patent publication WO/2012/175691, the contents of which is hereby incorporated by reference, particularly as shown at page 5.

In another embodiment, the anti-AXL antibody is an antibody as described in international patent publication WO/2012/175692, the contents of which is hereby incorporated by reference, particularly as shown at pages 4-5.

In another embodiment, the anti-AXL antibody is an antibody as described in international patent publication WO/2009/062690, the contents of which is hereby incorporated by reference.

In another embodiment, the anti-AXL antibody is an antibody as described in international patent publication WO/2010/130751, the contents of which is hereby incorporated by reference, particularly as shown at pages 1-17 (of 78).

In another embodiment, the anti-AXL antibody is an antibody as described in international patent publication WO/2013/064685, the contents of which is hereby incorporated by reference, particularly the 1613F12 antibody described therein as shown at, for example, Examples 6 to 8.

In another embodiment, the anti-AXL antibody is an antibody as described in international patent publication WO/2014/068139, the contents of which is hereby incorporated by reference, particularly the 110D7, 1003A2, and 1024G11 antibodies described therein as shown at, for example, Examples 6 to 8.

In another embodiment, the anti-AXL antibody is an antibody as described in international patent publication WO/2016/097370, the contents of which is hereby incorporated by reference, particularly the murine 10G5 and 10C9 antibodies described therein as shown at, for example, Examples 6 to 8.

In another embodiment, the anti-AXL antibody is an antibody as described in international patent publication WO/2017/220695, the contents of which is hereby incorporated by reference, particularly the humanized 10G5 antibody described therein as shown at, for example, SEQ ID NO. 1 to 10.

Where appropriate, the anti-AXL antibody may be a chimeric or humanised version of the antibodies described above.

### Preferred Embodiments

Preferably, the anti-AXL antibody is an antibody as described in WO/2016/097370, WO/2017/220695, WO/2015/193428, WO/2016/166296, WO/2015/193430, WO/2011/159980, WO/2013/064685, or WO/2014/068139 (the contents of each of which is hereby incorporated by reference), or a chimeric or humanised version of these antibodies where appropriate.

More preferably, the anti-AXL antibody is an antibody as described in WO/2016/097370, WO/2017/220695, WO/2011/159980, WO/2013/064685, or WO/2014/068139 (the contents of each of which is hereby incorporated by reference).

Most preferably the anti-AXL antibody is an antibody as described in WO/2017/220695, particularly the humanized 10G5 antibody described therein as shown at, for example, Examples 6 to 8.

In some embodiments, the anti-AXL antibody comprises the 6 CDRs having the sequences set out herein in SEQ ID Nos. 1 to 6.

In some embodiments, the anti-AXL antibody comprises the 6 CDRs having the sequences set out herein in SEQ ID Nos. 7 to 12.

In some embodiments, the anti-AXL antibody comprises a VH domain having the sequence set out herein in either one of SEQ ID Nos. 13 or 14. In some embodiments the antibody further comprises a VL domain having the sequence set out herein in either one of SEQ ID Nos. 15 or 16.

Immune Checkpoint Modulators (ICMs)

In the disclosed methods of treating an AXL-related disease, immune checkpoint inhibitors function to modulate the immune response to the AXL-related disease. This may be achieved in a number of ways, such as increasing the activity of stimulatory pathways and decreasing the activity of inhibitory pathways.

Immune responses to AXL-related diseases such as cancer are known to be able control tumour growth and in some cases lead to elimination of tumours. Therapeutic targeting of tumor immune regulators has resulted in the development of successful immunotherapeutic approaches for cancer treatment—for example agents blocking the activity of negative regulators of T cell immunity, such as a cytotoxic T-lymphocyte antigen 4 (CTLA-4) and programmed death receptor-1 (PD-1).

In some embodiments the immune checkpoint modulator (ICM) may be an immune checkpoint inhibitor (101). For example, an agent which acts at T cell co-inhibitory receptors, such as CTLA-4, PD-1, PD-L1, BTLA, TIM-3, VISTA, LAG-3, and TIGIT.

In some embodiments the immune checkpoint modulator (ICM) may be a T cell co-stimulatory agonist. For example, an agonist of a T-cell co-stimulatory receptor such as CD28, ICOS, 4-1BB, OX40, GITR, CD27, TWEAKR, HVEM, and TIM-1.

In some embodiments the immune checkpoint modulator (ICM) may act at dendritic cell co-stimulatory receptors, such as CD40 and 4-1BB.

In some embodiments, the immune checkpoint modulator may be an immune checkpoint modulating antibody. In some embodiments the immune checkpoint modulator may be selected from the group consisting of: anti-CTLA-4 antibodies, anti-PD-1 antibodies, anti-PD-L1 antibodies, anti-4-1BB antibodies, anti-OX-40 antibodies, anti-GITR antibodies, anti-CD27 antibodies, anti-CD28 antibodies, anti-CD40 antibodies, anti-LAGS antibodies, anti-ICOS antibodies, anti-TWEAKR antibodies, anti-HVEM antibodies, anti-TIM-1 antibodies, anti-TIM-3 antibodies, anti-VISTA antibodies, and anti-TIGIT antibodies.

In some preferred embodiments the immune checkpoint modulator may be selected from the group consisting of: anti-PD-1 antibodies, anti-PD-L1 antibodies, anti-CTLA-4 antibodies, anti-4-1BB antibodies, anti-OX-40 antibodies, anti-GITR antibodies, anti-CD27 antibodies, anti-CD40 antibodies, and anti-LAGS antibodies. In some particularly preferred embodiments the immune checkpoint modulator may be selected from the group consisting of: anti-PD-1 antibodies, anti-PD-L1 antibodies, and anti-CTLA-4 antibodies. In some most preferred embodiments, the immune checkpoint modulator may be an anti-PD-1 antibody.

Examples of ICMs suitable for use in the methods described herein include ipilimumab, tremelimumab, pembrolizumab, nivolumab, and urelumab, and those which can be identified by the drug candidate identifiers AMP-514/MEDI0680 (Medlmmune/AstraZeneca), MPDL3280A (Genentech/Roche), MED14736 (Medlmmune/AstraZeneca), MSB0010718C (EMD Serono), BMS-936559 (Bristol-Myers Squibb), PF-05082566 (Pfizer), MED16469 (Medlmmune/AstraZeneca), MED16383 (rOX40L; Medlmmune/AstraZeneca), MOXR0916 (Genentech/Roche), TRX518 (Tolerx), CDX-1127 (Celldex), CP-870,893 (Genentech/Roche), and BMS-986016 (Bristol-Myers Squibb) (preferably ipilimumab, tremelimumab, pembrolizumab, and nivolumab).

In some embodiments, the anti-GITR antibody or GITR agonist is selected from MEDI1873, TRX518, GWN323, MK-1248, MK 4166, BMS-986156 and INCAGN1876.

In some embodiments, the anti-0X40 antibody or 0X40 agonist is selected from MED10562, MED16383, MOXR0916, RG7888, OX40mAb24, INCAGN1949, GSK3174998, and PF-04518600.

In some preferred embodiments of the disclosed methods, two or more immune checkpoint modulators may be administered. Results have shown that an improved synergistic effect can be obtained when at least two different immune checkpoint (activity) modulators are employed, especially when such immune checkpoint (activity) modulators act at different cell receptor sub-types. For example, the combination of at least one immune checkpoint inhibitor and at least one T cell co-stimulatory receptor agonist or dendritic cell co-stimulatory receptor agonist.

Preferably, at least one of the two or more immune checkpoint (activity) modulators is an anti-CTLA-4 antibody, an anti-PD-1 antibody, or an anti-PD-L1 antibody. In particular, the combination of an anti-CTLA-4 antibody and an anti-PD-1 antibody has proven to be particularly effective.

In some preferred embodiments the two or more immune checkpoint (activity) modulators may include: (i) an immune checkpoint inhibitor, and (ii) a T cell co-stimulatory receptor agonist or a dendritic cell co-stimulatory receptor agonist. In some embodiments the two or more immune checkpoint (activity) modulators may include: (i) an anti-CTLA-4 antibody; and/or (ii) either an anti-PD-1 antibody or an anti-PD-L1 antibodies.

In some preferred embodiments the anti-CTLA-4 antibody is ipilimumab or tremelimumab.

In some preferred embodiments the anti-PD-1 antibody is pembrolizumab, nivolumab, spartalizumab Camrelizumab, Pidilizumab, or Cemiplimab. Preferably the anti-PD-1 antibody is pembrolizumab or nivolumab.

In some embodiments the anti-PDL1 antibody is Atezolizumab (CAS number 1380723-44-3), Avelumab (CAS number 1537032-82-8), or Durvalumab (CAS number 1428935-60-7).

In some embodiments the two or more immune checkpoint (activity) modulators may be administered concurrently. In other embodiments the two or more immune checkpoint (activity) modulators may be administered separately and/or sequentially in any order.

In some preferred embodiments the two or more immune checkpoint (activity) modulators may be ipilimumab and pembrolizumab.

Chemotherapeutic Agents

The disclosed methods of treating an AXL-related disease may comprise treatment with a combination of an AXL inhibitor (AXLi), an immune checkpoint modulator (ICM), and a chemotherapeutic agent. In the disclosed methods of treating an AXL-related disease, the chemotherapeutic agent may be any chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

In the disclosed methods of treating an AXL-related disease, the chemotherapeutic agents function to cause cell death of cancer cells (e.g. localised tumor cell death), the release of tumour antigens, and a subsequent immune response. Without wishing to be bound by theory, the authors believe that the chemotherapeutic agent induces cell death (apoptosis) and release of tumour antigens, upregulating IFN release and leading to a release of type I IFNs, which in turn activate AXL. Active AXL downregulates the IFN response and inhibits the immune response. AXL inhibition will therefore prevent inhibition of the immune response, and in turn potentiate the effect of the ICMs. Accordingly, the chemotherapeutic agent may be a chemotherapeutic agent which induces immunogenic cell death of cancer cells.

Examples of chemotherapeutic agents which may be used in the disclosed methods include: Lenalidomide (REVLIMID®, Celgene), Vorinostat (ZOLINZA®, Merck), Panobinostat (FARYDAK®, Novartis), Mocetinostat (MGCD0103), Everolimus (ZORTRESS®, CERTICAN®, Novartis), Bendamustine (TREAKISYM®, RIBOMUSTIN®, LEVACT®, TREANDA®, Mundipharma International), erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (P13K inhibitor, Semafore Pharmaceuticals), BEZ-235 (PI3K inhibitor, Novartis), XL-147 (P13K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gamma1I, calicheamicin omegal1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above.

Examples of chemotherapeutic agents used in the treatment of anal cancer include: Gardasil, Gardasil 9, Recombinant Human Papillomavirus (HPV) Nonavalent Vaccine, Recombinant Human Papillomavirus (HPV) Quadrivalent Vaccine.

Examples of chemotherapeutic agents used in the treatment of bladder cancer include: Atezolizumab, Avelumab, Balversa (Erdafitinib), Bavencio (Avelumab), Cisplatin, Doxorubicin Hydrochloride, Durvalumab, Erdafitinib, Imfinzi (Durvalumab), Keytruda (Pembrolizumab), Nivolumab, Opdivo (Nivolumab), Pembrolizumab, Tecentriq (Atezolizumab), Thiotepa, Valrubicin, and Valstar (Valrubicin).

Examples of chemotherapeutic agents used in the treatment of bone cancer include: Cosmegen (Dactinomycin), Dactinomycin, Denosumab, Doxorubicin Hydrochloride, Methotrexate, Trexall (Methotrexate), and Xgeva (Denosumab).

Examples of chemotherapeutic agents used in the treatment of brain tumors include: Afinitor (Everolimus), Afinitor Disperz (Everolimus), Avastin (Bevacizumab), Bevacizumab, BiCNU (Carmustine), Carmustine, Carmustine Implant, Everolimus, Gliadel Wafer (Carmustine Implant), Lomustine, Mvasi (Bevacizumab), Temodar (Temozolomide), and Temozolomide.

Examples of chemotherapeutic agents used in the treatment of breast cancer include: Abemaciclib, Abraxane (Paclitaxel Albumin-stabilized Nanoparticle Formulation), Ado-Trastuzumab Emtansine, Afinitor (Everolimus), Afinitor Disperz (Everolimus), Alpelisib, Anastrozole, Aredia (Pamidronate Disodium), Arimidex (Anastrozole), Aromasin (Exemestane), Atezolizumab, Capecitabine, Cyclophosphamide, Docetaxel, Doxorubicin Hydrochloride, Ellence (Epirubicin Hydrochloride), Enhertu (Farr-Trastuzumab Deruxtecan-nxki), Epirubicin Hydrochloride, Eribulin Mesylate, Everolimus, Exemestane, 5-FU (Fluorouracil Injection), Fam-Trastuzumab Deruxtecan-nxki, Fareston (Toremifene), Faslodex (Fulvestrant), Ferrara (Letrozole), Fluorouracil Injection, Fulvestrant, Gemcitabine Hydrochloride, Gemzar (Gemcitabine Hydrochloride), Goserelin Acetate, Halaven (Eribulin Mesylate), Herceptin Hylecta (Trastuzumab and Hyaluronidase-oysk), Herceptin (Trastuzumab), Ibrance (Palbociclib), Ixabepilone, Ixempra (Ixabepilone), Kadcyla (Ado-Trastuzumab Emtansine), Kisqali (Ribociclib), Lapatinib Ditosylate, Letrozole, Lynparza (Olaparib), Megestrol Acetate, Methotrexate, Neratinib Maleate, Nerlynx (Neratinib Maleate), Olaparib, Paclitaxel, Paclitaxel Albumin-stabilized Nanoparticle Formulation, Palbociclib, Pamidronate Disodium, Perjeta (Pertuzumab), Pertuzumab, Piqray (Alpelisib), Ribociclib, Talazoparib Tosylate, Talzenna (Talazoparib Tosylate), Tamoxifen Citrate, Taxol (Paclitaxel), Taxotere (Docetaxel), Tecentriq (Atezolizumab), Thiotepa, Toremifene, Trastuzumab, Trastuzumab and Hyaluronidase-oysk, Trexall (Methotrexate), Tykerb (Lapatinib Ditosylate), Verzenio (Abemaciclib), Vinblastine Sulfate, Xeloda (Capecitabine), and Zoladex (Goserelin Acetate).

Examples of chemotherapeutic agents used in the treatment of cervical cancer include: Avastin (Bevacizumab), Bevacizumab, Bleomycin Sulfate, Hycamtin (Topotecan Hydrochloride), Keytruda (Pembrolizumab), Mvasi (Bevacizumab), Pembrolizumab, Topotecan Hydrochloride.

Examples of chemotherapeutic agents used in the treatment of colon and rectal cancer include: Avastin (Bevacizumab), Bevacizumab, Camptosar (Irinotecan Hydrochloride), Capecitabine, Cetuximab, Cyramza (Ramucirumab), Eloxatin (Oxaliplatin), Erbitux (Cetuximab), 5-FU (Fluorouracil Injection), Fluorouracil Injection, Ipilimumab, Irinotecan Hydrochloride, Keytruda (Pembrolizumab), Leucovorin Calcium, Lonsurf (Trifluridine and Tipiracil Hydrochloride), Mvasi (Bevacizumab), Nivolumab, Opdivo (Nivolumab), Oxaliplatin, Panitumumab, Pembrolizumab, Ramucirumab, Regorafenib, Stivarga (Regorafenib), Trifluridine and Tipiracil Hydrochloride, Vectibix (Panitumumab), Xeloda (Capecitabine), Yervoy (Ipilimumab), Zaltrap (Ziv-Aflibercept), Ziv-Aflibercept.

Examples of chemotherapeutic agents used in the treatment of ovarian, fallopian tube, or primary peritoneal cancer include: Alkeran (Melphalan), Avastin (Bevacizumab), Bevacizumab, Carboplatin, Cisplatin, Cyclophosphamide, Doxorubicin Hydrochloride, Doxil (Doxorubicin Hydrochloride Liposome), Doxorubicin Hydrochloride Liposome, Gemcitabine Hydrochloride, Gemzar (Gemcitabine Hydrochloride), Hycamtin (Topotecan Hydrochloride), Lynparza (Olaparib),Melphalan, Niraparib Tosylate Monohydrate, Olaparib, Paclitaxel, Rubraca (Rucaparib Camsylate), Rucaparib Camsylate, Taxol (Paclitaxel), Thiotepa, Topotecan Hydrochloride, Zejula (Niraparib Tosylate Monohydrate).

Examples of chemotherapeutic agents used in the treatment of non-small cell lung cancer include: Abraxane (Paclitaxel Albumin-stabilized Nanoparticle Formulation), Afatinib Dimaleate, Afinitor (Everolimus), Afinitor Disperz (Everolimus), Alecensa (Alectinib), Alectinib, Alimta (Pemetrexed Disodium), Alunbrig (Brigatinib), Atezolizumab, Avastin (Bevacizumab), Bevacizumab, Brigatinib, Carboplatin, Ceritinib, Crizotinib, Cyramza (Ramucirumab), Dabrafenib Mesylate, Dacomitinib, Docetaxel, Doxorubicin Hydrochloride, Durvalumab, Entrectinib, Erlotinib Hydrochloride, Everolimus, Gefitinib, Gilotrif (Afatinib Dimaleate), Gemcitabine Hydrochloride, Gemzar (Gemcitabine Hydrochloride), Imfinzi (Durvalumab), Iressa (Gefitinib), Keytruda (Pembrolizumab), Lorbrena (Lorlatinib), Lorlatinib, Mechlorethamine Hydrochloride, Mekinist (Trametinib), Methotrexate, Mustargen (Mechlorethamine Hydrochloride), Mvasi (Bevacizumab), Navelbine (Vinorelbine Tartrate), Necitumumab, Nivolumab, Opdivo (Nivolumab), Osimertinib Mesylate, Paclitaxel, Paclitaxel Albumin-stabilized Nanoparticle Formulation, Paraplat (Carboplatin), Paraplatin (Carboplatin), Pembrolizumab, Pemetrexed Disodium, Portrazza (Necitumumab), Ramucirumab, Rozlytrek (Entrectinib), Tafinlar (Dabrafenib Mesylate), Tagrisso (Osimertinib Mesylate), Tarceva (Erlotinib Hydrochloride), Taxol (Paclitaxel), Taxotere (Docetaxel), Tecentriq (Atezolizumab), Trametinib, Trexall (Methotrexate), Vizimpro (Dacomitinib), Vinorelbine Tartrate, Xalkori (Crizotinib), Zykadia (Ceritinib).

Examples of chemotherapeutic agents used in the treatment of small cell lung cancer include: Afinitor (Everolimus), Atezolizumab, Doxorubicin Hydrochloride, Etopophos (Etoposide Phosphate), Etoposide, Etoposide Phosphate, Everolimus, Hycamtin (Topotecan Hydrochloride), Keytruda (Pembrolizumab), Mechlorethamine Hydrochloride, Methotrexate, Mustargen (Mechlorethamine Hydrochloride), Nivolumab, Opdivo (Nivolumab), Pembrolizumab, Tecentriq (Atezolizumab), Topotecan Hydrochloride, Trexall (Methotrexate).

Examples of chemotherapeutic agents used in the treatment of melanoma include: Aldesleukin, Binimetinib, Braftovi (Encorafenib), Cobimetinib, Cotellic (Cobimetinib), Dabrafenib Mesylate, Dacarbazine, Encorafenib, IL-2 (Aldesleukin), Imlygic (Talimogene Laherparepvec), Interleukin-2 (Aldesleukin), Intron A (Recombinant Interferon Alfa-2b), Ipilimumab, Keytruda (Pembrolizumab), Mekinist (Trametinib), Mektovi (Binimetinib), Nivolumab, Opdivo (Nivolumab), Peginterferon Alfa-2b, PEG-Intron (Peginterferon Alfa-2b), Pembrolizumab, Proleukin (Aldesleukin), Recombinant Interferon Alfa-2b, Sylatron (Peginterferon Alfa-2b), Tafinlar (Dabrafenib Mesylate), Talimogene Laherparepvec, Trametinib, Vemurafenib, Yervoy (Ipilimumab), Zelboraf (Vemurafenib).

Examples of chemotherapeutic agents used in the treatment of mesothelioma include: Alimta (Pemetrexed Disodium), and Pemetrexed Disodium.

Examples of chemotherapeutic agents used in the treatment of AML include: Arsenic Trioxide, Cerubidine (Daunorubicin Hydrochloride), Cyclophosphamide, Cytarabine, Daunorubicin Hydrochloride, Daunorubicin Hydrochloride and Cytarabine Liposome, Daurismo (Glasdegib Maleate), Dexamethasone, Doxorubicin Hydrochloride, Enasidenib Mesylate, Gemtuzumab Ozogamicin, Gilteritinib Fumarate, Glasdegib Maleate, Idamycin PFS (Idarubicin Hydrochloride), Idarubicin Hydrochloride, Idhifa (Enasidenib Mesylate), Ivosidenib, Midostaurin, Mitoxantrone Hydrochloride, Mylotarg (Gemtuzumab Ozogamicin), Rubidomycin (Daunorubicin Hydrochloride), Rydapt (Midostaurin), Tabloid (Thioguanine), Thioguanine, Tibsovo (Ivosidenib), Trisenox (Arsenic Trioxide), Venclexta (Venetoclax), Venetoclax, Vincristine Sulfate, Vyxeos (Daunorubicin Hydrochloride and Cytarabine Liposome), andXospata (Gilteritinib Fumarate).

Examples of chemotherapeutic agents used in the treatment of pancreatic cancer include: Abraxane (Paclitaxel Albumin-stabilized Nanoparticle Formulation), Afinitor (Everolimus), Erlotinib Hydrochloride, Everolimus, 5-FU (Fluorouracil Injection), Fluorouracil Injection, Gemcitabine Hydrochloride, Gemzar (Gemcitabine Hydrochloride), Irinotecan Hydrochloride Liposome, Lynparza (Olaparib), Mitomycin C, Olaparib, Onivyde (Irinotecan Hydrochloride Liposome), Paclitaxel Albumin-stabilized Nanoparticle Formulation, Sunitinib Malate, Sutent (Sunitinib Malate), and Tarceva (Erlotinib Hydrochloride).

Examples of chemotherapeutic agents used in the treatment of renal cancer include: Afinitor (Everolimus), Afinitor Disperz (Everolimus), Aldesleukin, Avastin (Bevacizumab), Avelumab, Axitinib, Bavencio (Avelumab), Bevacizumab, Cabometyx (Cabozantinib-S-Malate), Cabozantinib-S-Malate, Everolimus, IL-2 (Aldesleukin), Inlyta (Axitinib), Interleukin-2 (Aldesleukin), Ipilimumab, Keytruda (Pembrolizumab), Lenvatinib Mesylate, Lenvima (Lenvatinib Mesylate), Mvasi (Bevacizumab), Nexavar (Sorafenib Tosylate), Nivolumab, Opdivo (Nivolumab), Pazopanib Hydrochloride, Pembrolizumab, Proleukin (Aldesleukin), Sorafenib Tosylate, Sunitinib Malate, Sutent (Sunitinib Malate), Temsirolimus, Torisel (Temsirolimus), Votrient (Pazopanib Hydrochloride), and Yervoy (Ipilimumab).

Example of chemotherapeutic agents used to treat solid tumors anywhere in the body include: Entrectinib, Keytruda (Pembrolizumab), Larotrectinib Sulfate, Rozlytrek (Entrectinib), and Vitrakvi (Larotrectinib Sulfate).

Combination treatments are also included in the definition of “chemotherapeutic agent” used herein. Examples of combination treatments of chemotherapeutic agents include: gemcitabine-cisplatin, MVAC (methotrexate, vinblastine sulfate, doxorubicin hydrochloride, cisplatin), PCV (procarbazine hydrochloride, lomustine, vincristine sulfate), AC (doxorubicin hydrochloride, cyclophosphamide), AC-T (doxorubicin hydrochloride, cyclophosphamide, paclitaxel), CAF (cyclophosphamide, doxorubicin hydrochloride, fluorouracil), CMF (cyclophosphamide, methotrexate, fluorouracil), FEC (fluorouracil, epirubicin hydrochloride, cyclophosphamide), TAC (docetaxel, doxorubicin hydrochloride, cyclophosphamide), CAPDX (capecitabine, oxaliplatin), FOLFIRI (leucovorin calcium, fluorouracil, irinotecan hydrochloride), FOLFIRI-Bevacizumab, FOLFIRI-Cetuximab, FOLFOX (leucovorin calcium, fluorouracil, oxaliplatin), FU-LV (fluorouracil, leucovorin calcium), XELIRI (capecitabine, irinotecan hydrochloride), XELOX (capecitabine, oxaliplatin), TPF (docetaxelm, cisplatin, fluorouracil), ABVD (doxorubicin hydrochloride, bleomycin, vinblastine sulfate, dacarbazine), ABVE (doxorubicin hydrochloride, bleomycin, vincristine sulfate, etoposide phosphate), ABVE-PC (doxorubicin hydrochloride, bleomycin, vincristine sulfate, etoposide phosphate, prednisone, cyclophosphamide), BEACOPP (bleomycin, etoposide phosphate, doxorubicin hydrochloride, cyclophosphamide, vincristine sulfate, procarbazine hydrochloride, prednisone), COPDAC (cyclophosphamide, vincristine sulfate, prednisone, dacarbazine), COPP cyclophosphamide, vincristine sulfate, procarbazine hydrochloride, prednisone), COPP-ABV cyclophosphamide, vincristine sulfate, procarbazine hydrochloride, prednisone, doxorubicin hydrochloride, bleomycin, vinblastine sulfate), ICE (ifosfamide, carboplatin, etoposide phosphate), MOPP (mechlorethamine hydrochloride, vincristine sulfate, procarbazine hydrochloride, prednisone), OEPA (vincristine sulfate, etoposide phosphate, prednisone, doxorubicin hydrochloride), OPPA (vincristine sulfate, procarbazine hydrochloride, prednisone, doxorubicin hydrochloride), STANFORD V (mechlorethamine hydrochloride, doxorubicin hydrochloride, vinblastine sulfate, vincristine sulfate, bleomycin, etoposide phosphate, prednisone), VAMP (vincristine sulfate, doxorubicin hydrochloride, methotrexate, prednisone), hyper-CVAD (cyclophosphamide, vincristine sulfate, doxorubicin hydrochloride, dexamethasone), ADE (cytarabine, daunorubicin hydrochloride, etoposide phosphate), chlorambucil-prednisone, CVP (cyclophosphamide, vincristine sulfate, prednisone), carboplatin-taxol, PAD (bortezomib, doxorubicin hydrochloride, dexamethasone), BuMel (busulfan, melphalan hydrochloride), CEM (carboplatin, etoposide phosphate, melphalan hydrochloride), CHP (doxorubicin, prednisone, cyclophosphamide), CHOP (doxorubicin, prednisone, cyclophopsphamide, vincristine), EPOCH (etoposide phosphate, prednisone, vincristine sulfate, cyclophosphamide, doxorubicin hydrochloride) ICE (ifosfamide, carboplatin, etoposide phosphate) R-CHOP (rituximab, doxorubicin, prednisone, cyclophopsphamide, vincristine), R-CVP (rituximab, cyclophosphamide, vincristine sulfate, prednisone) R-EPOCH (rituximab, etoposide phosphate, prednisone, vincristine sulfate, cyclophosphamide, doxorubicin hydrochloride), R-ICE (rituximab, ifosfamide, carboplatin, etoposide phosphate), BEP (bleomycin, etoposide phosphate, cisplatin), JEB (carboplatin, etoposide phosphate, bleomycin), PEB (cisplatin, etoposide phosphate, bleomycin) VAC (vincristine sulfate, dactinomycin, cyclophosphamide), VeIP (vinblastine sulfate, ifosfamide, cisplatin), Carboplatin/Doxil, Carboplatin/Gemcitabine, Carboplatin/Topotecan, Taxol/Avastin, FOLFIRINOX (leucovorin calcium, fluorouracil, irinotecan hydrochloride, oxaliplatin), Gemcitabine-cisplatin, gemcitabine oxaliplatin, OFF (oxaliplatin, fluorouracil, leucovorin calcium), CEV (carboplatin, etoposide phosphate, vincristine sulfate), and VIP (etoposide, ifosfamide, cisplatin).

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), ofatumumab (ARZERRA®, GSK), pertuzumab (PERJETATM, OMNITARGTM, 2C4, Genentech), trastuzumab (HERCEPTIN®, Genentech), tositumomab (Bexxar, Corixia), MDX-060 (Medarex).

Also included in the definition of “chemotherapeutic agent” are antibody drug conjugates, in particular AXL antibody drug conjugates. For example, gemtuzumab ozogamicin (MYLOTARG®, Wyeth), enapotamab vedotin (FluMax®-AXL-ADC, Genrnab), CAB-AXL-ADC (BioAtla).

Humanized monoclonal antibodies with therapeutic potential as chemotherapeutic agents in combination with the conjugates of the disclosure include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Certain chemotherapeutic agents are known to influence pathways involved in the immune response. For example, the class of cytotoxic chemotherapeutic agents called anthracyclines are known to induce a Type I Interferon response mimicking immune responses to viruses, and the clinical response to antracycline therapy correlates with a Type I IFN gene signature (Sistigue et al 2014; Zitvogel et al, 2015). As AXL serves as a key checkpoint for interferon (IFN) signaling, stimulating IFN signaling in the context of AXL-inhibition could lead to enhanced anticancer T cell responses during immune checkpoint inhibition.

Accordingly, in some embodiments the chemotherapeutic agent may be a chemotherapeutic agent which induces an immune response in the subject. In some embodiments the chemotherapeutic agent may be a chemotherapeutic agent which induces a type I interferon response in the subject.

In some preferred embodiments, the chemotherapeutic agent may be an anthracycline. In some such embodiments the chemotherapeutic agent may be selected from the group consisting of: Daunorubicin, Doxorubicin, Epirubicin, Idarubicin, Mitoxantrone, and Valrubicin. In some particularly preferred embodiments, the chemotherapeutic agent may be doxorubicin.

Radiotherapy

The disclosed methods of treating an AXL-related disease may comprise treatment with a combination of an AXL inhibitor (AXLi), an immune checkpoint modulator (ICM), and radiotherapy. In the disclosed methods of treating an AXL-related disease, the terms “radiation therapy” or “radiotherapy” may refer to the medical use of ionizing radiation as part of cancer treatment to control or eradicate malignant cells. Radiotherapy may be used for curative, adjuvant, or palliative treatment. Suitable types of radiotherapy include conventional external beam radiotherapy, stereotactic radiation therapy (e.g., Axesse, Cyberknife, Gamma Knife, Novalis, Primatom, Synergy, X-Knife, TomoTherapy or Trilogy), Intensity-Modulated Radiation Therapy, particle therapy (e.g., proton therapy), brachytherapy, delivery of radioisotopes, intraoperative radiotherapy, Auger therapy, Volumetric modulated arc therapy (VMAT), Virtual simulation, 3-dimensional conformal radiation therapy, and intensity-modulated radiation therapy.

In some embodiments, radiatiotherapy uses high-energy radiation to shrink tumors and kill cancer cells. The radiation may be, for example, X-rays, gamma rays, or charged particles. Modes of cell killing through radiation include DNA damage either directly or by creating free radicals within cells that in turn damage DNA.

Radiation may be delivered by a machine outside the body (external-beam radiation therapy), or may come from radioactive material placed in the body near cancer cells (internal radiation therapy, also called brachy therapy). In one example of systemic radiation therapy, radioactive substances, such as radioactive iodine, are used which travel in the blood to kill cancer cells.

Preferably, the radiotherapy may be administered in a regime designed to minimize any immunosuppressive effects of the radiation. For example, preclinical evidence indicates high radiation doses above 12-18 Gy result in an attenuation of tumor immunogenicity (Vanpouille-Box C., et al., Nat Commun 2017; 8: 15618). In addition, it is known that circulating lymphocytes are particularly radiosensitive (see Yovino S., et al., Cancer Invest 2013; 31: 140-144); this indicates radiotherapy regimes aimed at stimulating an anti-tumour immune response should aim to minimise both (1) the amount of vasculature exposed in each treatment, and (2) the number of exposures in the treatment regime.

Radiation dosages may be fractionated and administered in sequence; for example, on consecutive days until the total desired radiation dose is delivered.

Methods of Prognosis

The present disclosure is based on the finding that combination therapies comprising an AXL inhibitor (such as Bemcentinib, BGB324) and an immune checkpoint modulator (such as the PD1 inhibitor pembrolizumab) are effective in non-small cell lung cancer patients, including in patients with STK11 mutations. This was unexpected in view of literature reports that subjects with STK11 mutations are resistant to treatment with PD1 inhibitors (see, e.g., Skoulidis et al, 2018).

Thus, in addition to the methods of selecting a subject and methods of treating a subject described elsewhere herein, the present disclosure also provides method of prognosing susceptibility of a subject to treatment with the combination therapies disclosed herein. Such methods include:

A method of prognosing susceptibility of a subject to treatment with a combination of an AXL inhibitor (AXLi) and an immune checkpoint modulator (ICM), the method comprising: determining: i) the presence or absence of a STK11 mutation; and/or ii) the level of STK11 activity or expression in the subject or a sample derived from the subject; wherein the presence of a STK11 mutation and/or a modified level of STK11 activity or expression is indicative of susceptibility to treatment with a combination of an AXL inhibitor (AXLi) and an immune checkpoint modulator (ICM). In some embodiments, the modified STK11 activity or expression is decreased STK11 activity or expression.

A method of prognosing susceptibility of a subject to treatment in a method of treatment of the disclosure, the method comprising: determining: i) the presence or absence of a STK11 mutation; and/or ii) the level of STK11 activity or expression in the subject or a sample derived from the subject; wherein the presence of a STK11 mutation and/or a modified level of STK11 activity or expression is indicative of susceptibility to treatment in a method of treatment of the disclosure. In some embodiments, the modified STK11 activity or expression is decreased STK11 activity or expression.

A method of prognosing susceptibility of a subject to treatment with a combination of an AXL inhibitor (AXLi) and an immune checkpoint modulator (ICM), the method comprising: determining: i) the presence or absence of a STK11 mutation; ii) the presence or absence of a STK11IP mutation; iii) the level of STK11 activity or expression in the subject or a sample derived from the subject; and/or iv) the level of STK11IP activity or expression in the subject or a sample derived from the subject; wherein the presence of a STK11 mutation; the presence of a STK11IP mutation; a modified level of STK11 activity or expression; and/or an increased level of STK11IP activity or expression is indicative of susceptibility to treatment with a combination of an AXL inhibitor (AXLi) and an immune checkpoint modulator (ICM).

A method of prognosing susceptibility of a subject to treatment with a combination of an AXL inhibitor (AXLi), an immune checkpoint modulator (ICM), and a chemotherapeutic agent and/or radiotherapy, the method comprising: determining: i) the presence or absence of a STK11 mutation; ii) the presence or absence of a STK11IP mutation; iii) the level of STK11 activity or expression in the subject or a sample derived from the subject; and/or iv) the level of STK11IP activity or expression in the subject or a sample derived from the subject; wherein the presence of a STK11 mutation; the presence of a STK11IP mutation; a modified level of STK11 activity or expression; and/or an increased level of STK11IP activity or expression is indicative of susceptibility to treatment with a combination of an AXL inhibitor (AXLi), an immune checkpoint modulator (ICM), and a chemotherapeutic agent and/or radiotherapy. In some embodiments, the modified STK11 activity or expression is decreased STK11 activity or expression.

A method of prognosing susceptibility of a subject to treatment in a method of treatment of the disclosure, the method comprising: determining: i) the presence or absence of a STK11 mutation; ii) the presence or absence of a STK11IP mutation; iii) the level of STK11 activity or expression in the subject or a sample derived from the subject; and/or iv) the level of STK11IP activity or expression in the subject or a sample derived from the subject; wherein the presence of a STK11 mutation; the presence of a STK11IP mutation; a modified level of STK11 activity or expression; and/or an increased level of STK11IP activity or expression is indicative of susceptibility to treatment in a method of treatment of the disclosure. In some embodiments, the modified STK11 activity or expression is decreased STK11 activity or expression.

In some embodiments, the methods of prognosing of the disclosure may further comprise: determining: i) the presence or absence of a KRAS mutation; and/or ii) the level of KRAS activity or expression in the subject or a sample derived from the subject; wherein the presence of a KRAS mutation and/or an increased level of KRAS activity or expression is indicative of susceptibility to treatment.

In some embodiments, the methods may further comprise: determining: i) the presence or absence of a p53 mutation; and/or ii) the level of p53 activity or expression in the subject or a sample derived from the subject; wherein the presence of a p53 mutation and/or an decreased level of p53 activity or expression is indicative of susceptibility to treatment.

In some embodiments, the methods may further comprise: determining the level of AXL activity or expression in the subject or a sample derived from the subject; wherein an increased level of AXL activity or expression is indicative of susceptibility to treatment.

The disclosed methods of prognosing susceptibility of a subject to treatment with a combination treatment of the disclosure may comprise testing subjects in a method of selecting a subject for treatment as described in detail elsewhere herein. Subjects determined to be susceptible to treatment may subsequently be treated in a method of treatment according to the present disclosure.

Compositions, Uses, and Kits

In addition to the methods of selecting a subject for treatment, and methods of treating an AXL-related disease described in detail above, the present disclosure provides agents and reagents, as well as compositions and kits comprising these agents and reagents, for use in the disclosed methods.

Accordingly, the present disclosure provides an AXL inhibitor, an immune checkpoint modulator (ICM), and a chemotherapeutic agent, for use in a method of treatment according to the present disclosure. Also provided is: an AXL inhibitor for use in a method of treatment according to the present disclosure; an immune checkpoint modulator (ICM) for use in a method of treatment according to the present disclosure; a chemotherapeutic agent for use in a method of treatment according to the present disclosure; an AXL inhibitor and an immune checkpoint modulator (ICM) for use in a method of treatment according to the present disclosure; an AXL inhibitor and a chemotherapeutic agent for use in a method of treatment according to the present disclosure; and, an immune checkpoint modulator (ICM) and a chemotherapeutic agent for use in a method of treatment according to the present disclosure.

Thus, the present disclosure provides an AXL inhibitor, an immune checkpoint modulator (ICM), and a chemotherapeutic agent, for use in a method of treatment according to the present disclosure. Also provided is: an AXL inhibitor for use in a method of treatment according to the present disclosure; an immune checkpoint modulator (ICM) for use in a method of treatment according to the present disclosure; a chemotherapeutic agent for use in a method of treatment according to the present disclosure; an AXL inhibitor and an immune checkpoint modulator (ICM) for use in a method of treatment according to the present disclosure; an AXL inhibitor and a chemotherapeutic agent for use in a method of treatment according to the present disclosure; and, an immune checkpoint modulator (ICM) and a chemotherapeutic agent for use in a method of treatment according to the present disclosure. Also provided is radiotherapy, for use in a method of treatment according to the present disclosure.

Also provided is the use of an AXL inhibitor, an immune checkpoint modulator (ICM), and a chemotherapeutic agent in the manufacture of a medicament for treating a disorder in a subject, wherein the treatment comprises a method of treatment according to the present disclosure. Also provided is: use of an Axl inhibitor in the manufacture of a medicament for treating a disorder in a subject, wherein the treatment comprises a method of treatment according to the present disclosure; use of an immune checkpoint modulator (ICM) in the manufacture of a medicament for treating a disorder in a subject, wherein the treatment comprises a method of treatment according to the present disclosure; use of a chemotherapeutic agent in the manufacture of a medicament for treating a disorder in a subject, wherein the treatment comprises a method of treatment according to the present disclosure; use of an AXL inhibitor and an immune checkpoint modulator (ICM) in the manufacture of a medicament for treating a disorder in a subject, wherein the treatment comprises a method of treatment according to the present disclosure; use of an AXL inhibitor and a chemotherapeutic agent in the manufacture of a medicament for treating a disorder in a subject, wherein the treatment comprises a method of treatment according to the present disclosure; and, use of an immune checkpoint modulator (ICM) and a chemotherapeutic agent in the manufacture of a medicament for treating a disorder in a subject, wherein the treatment comprises a method of treatment according to the present disclosure.

The present disclosure also provides a kit comprising an AXL inhibitor, an immune checkpoint modulator (ICM), and/or a chemotherapeutic agent, for use in a method of treating an Axl-related disease as disclosed herein.

The present disclosure also provides: a reagent for detecting activity, expression, or amount of STK11, STK11IP, KRAS, or p53, for use in a method of selecting a subject for treatment according to the present disclosure; a kit comprising 1, 2, 3, 4, or more reagents for detecting activity, expression, or amount of one or more of STK11, STK11IP, KRAS, or p53, for use in a method of selecting a subject for treatment according to the present disclosure; and, use of such reagents and kits in the manufacture of a kit or test for use in a method of selecting a subject for treatment according to the present disclosure.

In some embodiments, each reagent for detecting may be a specific binding member which is selective for STK11, STK11IP, KRAS, or p53. In some embodiments, the reagent for detecting may be an antibody, a nucleic acid probe, or a QPCR primer.

The present disclosure also provides: a reagent for detecting CD8⁺ cells having TCF′ activity or expression, for use in a method of selecting a subject for treatment according to the present disclosure; a kit comprising 1, 2, 3, 4, or more reagents for detecting CD8⁺ cells having TCF1 activity or expression for use in a method of selecting a subject for treatment according to the present disclosure; and, use of such reagents and kits in the manufacture of a kit or test for use in a method of selecting a subject for treatment according to the present disclosure. The reagents may include, for example, antibodies (or other affinity reagents) against CD8 or TCF1.

The present disclosure also provides: a reagent for detecting the population of desired T cells in a subject, for use in a method of selecting a subject for treatment according to the present disclosure; a kit comprising 1, 2, 3, 4, or more reagents for detecting CD8+ cells having TCF1 activity or expression for use in a method of selecting a subject for treatment according to the present disclosure; and, use of such reagents and kits in the manufacture of a kit or test for use in a method of selecting a subject for treatment according to the present disclosure. The reagents may include, for example, antibodies (or other affinity reagents) against markers expressed by the desired T cells, such as CD8 or TCF1.

Compositions according to the present disclosure are preferably pharmaceutical compositions. Pharmaceutical compositions according to the present disclosure, and for use in accordance with the present disclosure, may comprise, in addition to the active ingredient(s), (i.e. AXL inhibitors, immune checkpoint modulators (ICM), and/or chemotherapeutic agents), a pharmaceutically acceptable excipient, carrier, buffer, stabiliser or other materials well known to those skilled in the art. Such materials should be non-toxic and should not interfere with the efficacy of the active ingredient(s). The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatine.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, and Lactated Ringer's Injection. Preservatives, stabilisers, buffers, antioxidants and/or other additives may be included, as required.

In some embodiments of the disclosure, the disclosed AXL inhibitor, ICM, chemotherapeutic agent, AXL inhibitor+ICM combination, ICM+chemotherapeutic agent combination, or AXL inhibitor+ICM+chemotherapeutic agent combination, may be comprised in a pharmaceutical composition, optionally further comprising a pharmaceutically acceptable excipient.

The present disclosure also provides such compositions for use in a method of treating an Axl-related disease, and use of such compositions in the manufacture of a medicament for treating a disorder in a subject, wherein the treatment comprises a method of treatment according to the present disclosure.

Subjects

The terms “subject”, “patient” and “individual” are used interchangeably herein. The subject may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human. The subject may be any of its forms of development, for example, a foetus. In preferred embodiments, the subject is a human.

Dosage

It will be appreciated by one of skill in the art that appropriate dosages of the AXL inhibitors, immune checkpoint modulators (ICM), chemotherapeutic agents, and compositions comprising these active elements, can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

In some cases, the dosage of AXL inhibitor may be determined by the expression of a first marker observed in a sample obtained from the subject. Thus, the level or localisation of expression of the first marker in the sample may be indicative that a higher or lower dose of AXL inhibitor is required. For example, a high expression level of the first marker may indicate that a higher dose of AXL inhibitor would be suitable. In some cases, a high expression level of the first marker may indicate a more aggressive therapy.

In some cases, the dosage of the ICM may be determined by the expression of a second marker observed in a sample obtained from the subject. Thus, the level or localisation of expression of the second marker in the sample may be indicative that a higher or lower dose of ICM is required. For example, a high expression level of the second marker may indicate that a higher dose of ICM would be suitable. In some cases, a high expression level of the second marker may indicate a more aggressive therapy.

In some cases, the dosage of the chemotherapeutic agent may be determined by the expression of a third marker observed in a sample obtained from the subject. Thus, the level or localisation of expression of the third marker in the sample may be indicative that a higher or lower dose of chemotherapeutic agent is required. For example, a high expression level of the third marker may indicate that a higher dose of chemotherapeutic agent would be suitable. In some cases, a high expression level of the third marker may indicate a more aggressive therapy.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of each active compound is in the range of about 100 ng to about 25 mg (more typically about 1 pg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

In some embodiments, each active compound is administered to a human subject according to the following dosage regime: about 100 mg, 3 times daily. In other embodiments, each active compound is administered to a human subject according to the following dosage regime: about 150 mg, 2 times daily. In other embodiments, each active compound is administered to a human subject according to the following dosage regime: about 200 mg, 2 times daily. In yet other embodiments, each active compound is administered to a human subject according to the following dosage regime: about 50 or about 75 mg, 3 or 4 times daily. In other embodiments, each active compound is administered to a human subject according to the following dosage regime: about 100 or about 125 mg, 2 times daily.

Antibodies

The term “antibody” herein is used in the broadest sense and specifically covers monoclonal antibodies, polyclonal antibodies, dimers, multimers, multispecific antibodies (e.g., bispecific antibodies), intact antibodies (also described as “full-length” antibodies) and antibody fragments, so long as they exhibit the desired biological activity, for example, the ability to bind a first target protein (Miller et al (2003) Jour. of Immunology 170:4854-4861). Antibodies may be murine, human, humanized, chimeric, or derived from other species such as rabbit, goat, sheep, horse or camel.

An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by Complementarity Determining Regions (CDRs) on multiple antibodies. Each antibody that specifically binds to a different epitope has a different structure. Thus, one antigen may have more than one corresponding antibody. An antibody may comprise a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1m1, G1m2, G1m3, non-G1m1 [that, is any allotype other than G1m1], G1m17, G2m23, G3m21, G3m28, G3m11, G3m5, G3m13, G3m14, G3m10, G3m15, G3m16, G3m6, G3m24, G3m26, G3m27, A2m1, A2m2, Km1, Km2 and Km3) of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

Embodiments of the Disclosure

Certain specifically contemplated embodiments of the disclosure are as follows:

In some preferred embodiments a subject is selected for treatment with a method of selecting a subject according to the disclosure (such as a method according to any one of statements 101-194 below) and is subsequently treated in a method of treating an AXL-related disease according to the disclosure (such as a method according to any one of statements 201-285 below).

In some preferred embodiments, a subject who has been selected for treatment with a method of selecting a subject according to the disclosure (such as a method according to any one of statements 101-194 below) is treated in a method of treating an AXL-related disease according to the disclosure (such as a method according to any one of statements 201-285 below). In some preferred embodiments, susceptibility of a subject to treatment with a combination of an AXL inhibitor (AXLi) and an immune checkpoint modulator (ICM) is determined in a prognostic method of the disclosure (such as a method according to any one of statements 601-608 below), and a subject determined to be susceptible to treatment is treated in a method of treating an AXL-related disease according to the disclosure (such as a method according to any one of statements 201-285 below).

In some preferred embodiments the AXL-related disease is cancer, such as breast cancer, lung cancer, non-small-cell lung cancer, melanoma, mesothelioma, acute myeloid leukemia (AML), myelodysplatic syndrome (MDS), pancreas cancer, kidney cancer, urothelial carcinoma, and glioblastoma. In some particularly preferred embodiments the cancer is lung cancer, more preferably non-small cell lung cancer.

In some preferred embodiments, modified STK11 activity or expression is assessed by determining the presence or absence of a STK11 mutation and/or a STK11IP mutation.

In some preferred embodiments the AXLi is Bemcentinib, and the ICM is a PD-1/PD-L1 inhibitor (such as Pembrolizumab or Durvalumab) and/or a CTLA-4 inhibitor (such as Ipilimumab or tremelimumab). In some preferred embodiments the chemotherapeutic agent is an anthracycline (such as doxorubicin).

In some preferred embodiments: the AXLi is Bemcentinib, the ICM is a PD-1/PD-L1 inhibitor (such as Pembrolizumab or Durvalumab) and/or a CTLA-4 inhibitor (such as Ipilimumab or tremelimumab); and, the AXL-related disease is cancer (such as lung cancer, preferably non-small cell lung cancer). In some such embodiments, the chemotherapeutic agent is an anthracycline (such as doxorubicin).

In some preferred embodiments: the AXLi is administered prior to administration of the immune checkpoint modulator (ICM). In some preferred embodiments: the AXLi is administered prior to administration of the chemotherapeutic agent and prior to administration of the immune checkpoint modulator (ICM); and, the chemotherapeutic agent is administered prior to administration of the immune checkpoint modulator (ICM).

In some preferred embodiments: the AXLi and ICM are administered to the subject no more than 3 weeks apart, preferably no more than 1 week apart. In some preferred embodiments: the AXLi and chemotherapeutic agent are administered to the subject no more than 3 weeks apart, preferably no more than 1 week apart; and, the AXLi and ICM are administered to the subject no more than 3 weeks apart, preferably no more than 1 week apart.

In some preferred embodiments: Bemcentinib and doxorubicin are administered to the subject no more than 3 weeks apart, preferably no more than 1 week apart; and, Bemcentinib and PD-1/PD-L1 inhibitor and CTLA-4 inhibitor are administered to the subject no more than 3 weeks apart, preferably no more than 1 week apart.

In some preferred embodiments: the AXLi is administered to the subject daily; the ICM is administered to the subject every 3 weeks; and, the chemotherapeutic agent is administered to the subject every 3 weeks.

In some preferred embodiments: Bemcentinib is administered to the subject daily; PD-1/PD-L1 inhibitor and CTLA-4 inhibitor are administered to the subject every 3 weeks; and, doxorubicin is administered to the subject every 3 weeks.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### FIGURES

FIG. 1 shows disease status and time on treatment in patients evaluable for cAXL. The level of PD-L1 is indicated for each patient as is the cAXL status (+ or −). Response criteria is based on the RECIST v1.1 criteria for solid tumors. This is based on the RECIST v1.1 criteria (for solid tumors) and is assigned in the eCRF by the investigator. Partial Response (PR) indicates at least a 30% decrease in the sum of the diameters of TLs since baseline. Stable Disease (SD) indicates neither sufficient shrinkage to qualify for PR nor sufficient increase to qualify for PD. Progressive Disease (PD) At least a 20% increase in the sum of diameters of TLs and an absolute increase of at least 5 mm, taking as reference the smallest sum on study (this includes the baseline sum if that is the smallest on study)

FIG. 2 shows a survival graph of the progression free survival (PFS) of patients on treatment with Bemcentinib and Pembrolizumab. The PFS is the length of time during and after treatment where the patient lives with the disease but does not get worse.

FIG. 3 Shows the tumor volume by day in KP9-1 tumors in mice treated with a placebo or anti-PD-1 therapy.

FIG. 4 Shows the tumor volume by day in KP9-3 tumors in mice treated with a placebo or anti-PD-1 therapy.

FIG. 5 Shows the tumor volume by day in KP9-3 tumors in mice treated with a placebo, anti-PD-1 therapy, Bemcentinib therapy, or a combination of anti-PD-1 therapy and Bemcentinib.

FIG. 6 Shows the tumor volume by day in A549 Xenografts in Humanized mice treated with a placebo, anti-PD-1 therapy, Bemcentinib therapy, or a combination of anti-PD-1 therapy and Bemcentinib.

FIG. 7 (A) Shows tumor growth in C57BL/6J mice (n=5) that were inoculated with 1×10⁶ KP9-3 (left) or KPL9-3-1 (right) tumor cells and treated with PD-1 (10 mg/kg, day 7, 10, 14). Tumor growth was measured every 3 days (B) Shows the abundance of TCF1⁺PD-1⁺ cells among gated CD8⁺ tumor infiltrating lymphocytes (TILs) (per mm³ of tumor) at day 14 after tumor inoculation. (B) Shows a volcano plot (left): Points for up- (red) and down- (blue) regulated genes in central memory T cell cluster with Tcf7 are highlighted. The expression level of Tcf7 in CD8⁺ T cells of KP9-3 (blue) and KPL9-3-1 (red) are compared and visualized through violin plot (right). (C-D) Abundance of TCF1⁺PD-1⁺ cells among gated CD8⁺ tumor infiltrating lymphocytes (TILs) (per mm³ of tumor) at day 14 after tumor inoculation.

FIG. 8 (A) shows tumor growth in C57BL/6J mice (n=5) inoculated with 1×10⁶ KPL9-3-1 tumor cells and treated with either BGB324 (50 mg/kg, twice daily), PD-1 (10 mg/kg, day 7, 10, 14), or in combination started at day 7 after tumor inoculation. Control group were treated with control IgG (10 mg/kg) and vehicle (50 mg/kg). Tumor growth was measured every 3 days. (B) Shows the abundance of TCF1⁺PD-1⁺ cells among gated CD8⁺ TILs (per mm³ of tumor) at day7 after treatment started (day 14 after tumor inoculation). (C) Shows staining of TCF1⁺ (orange) expressing CD8⁺ (green) T cells in each treatment group of KPL9-3-1 tumors. (D) Shows the treatment preference of stem, clonal expanded and exhaustive effector CD8⁺ T cells. The ratio of observed cell numbers to random expectation estimated by Roie index through chi-square test. +++ (Roien, P<0.05) represents highly enriched, ++ (1.2≤R_{o/e}<3, P<0.05) represents enriched, + (0.8≤R_{o/e}<1.2, P<0.05) represents weakly enriched, − (0<R_{o/e}<0.8, P<0.05) represents not significant or reduced. (E) Shows shared clonotypes of TCR between clusters in CD8⁺ T cells detected by sc-TCRseq (top).

FIG. 9 (A) shows the tumor growth of KPL9-3-1 tumor cells (40 g) treated with BGB324 (40 nM) for 24 h.

(B) Shows tumor growth of C57BL/6J mice (n=5) inoculated with 1×10⁶ KPL9-3-1 tumor cells with interferon alpha receptor blocking antibody treated with either BGB324 (50 mg/kg, twice daily), and PD-1 (10 mg/kg, day 7, 10, 14), or corresponding IgG and vehicle started at day 7 after tumor inoculation. Tumor growth was measured every 3 days.(C-D) shows the abundance of TCF1⁺PD-1⁺ cells among gated CD8⁺ TILs (per mm³ of tumor) at day 7 after treatment started. (E) Shows the mean fluorescent intensities (MFIs) of TCF1⁺ cells among gated CD8⁺ OT-1 cells. Bone marrow dendritic cells (BMDC) were co-cultured with isolated CD8⁺ T cells stimulated with ovalbumin.(F) Shows MFIs of TCF1⁺ cells among gated CD8⁺ OT-1 cells. (H) Shows the abundance of TCF1⁺PD-1⁺ cells among gated CD8⁺ TILs (per mm³ of tumor) at 48 h after with or without IFNα (200 ng) intratumoral injection.

SEQUENCES
[10C9 Heavy CDR1]
SEQ ID NO. 1
DYNFTRYYIH
[10C9 Heavy CDR2]
SEQ ID NO. 2
WIYPGTGDSKYNEKFKG
[10C9 Heavy CDR3]
SEQ ID NO. 3
NGNYWYFDV
[10C9 Light CDR1]
SEQ ID NO. 4
RSSKSLLHSNGNTYLY
[10C9 Light CDR2]
SEQ ID NO. 5
RMSNLAS
[10C9 Light CDR3]
SEQ ID NO. 6
MQHREYPFT
[10G5 Heavy CDR1]
SEQ ID NO. 7
GYSFTDFYIN
[10G5 Heavy CDR2]
SEQ ID NO. 8
RIFPGGDNTYYNEKFKG
[10G5 Heavy CDR3]
SEQ ID NO. 9
RGLYYAMDY
[10G5 Light CDR1]
SEQ ID NO. 10
RSSQSLVHSNGIPYLH
[10G5 Light CDR2]
SEQ ID NO. 11
RVSNRFS
[10G5 Light CDR3]
SEQ ID NO. 12
SQGTHVPPT
[hu10G5 VH(GH1)]
SEQ ID NO. 13
EVQLVQSGAGLVQPGGSVRLSCAASGYSFTDFYIN
WVRQAPGKGLEWIARIFPGGDNTYYNEKFKGRFTL
SADTSSSTAYLQLNSLRAEDTAVYYCARRGLYYAM
DYWGQGTLVTVSS
[hu10G5 VH(GH2)]
SEQ ID NO. 14
EVQLVESGGGLVQPGGSLRLSCAASGYSFTDFYIN
WVRQAPGKGLEWVARIFPGGDNTYYNEKFKGRFTL
SADTSKSTAYLQMNSLRAEDTAVYYCARRGLYYAM
DYWGQGTLVTVSS
[hu10G5 VL(GL1)]
SEQ ID NO. 15
DIQMTQSPSSLSASVGDRVTITCRSSQSLVHSNGI
PYLHWYQQKPGKAPKLLIYRVSNRFSGVPSRFSGS
GSGTDFTLTISSLQPEDFATYYCSQGTHVPPTFGQ
GTKVEIK
[hu10G5 VL(GL2)]
SEQ ID NO. 16
DIQMTQSPSSLSASVGDRVTITCRSSQSLVHSNGI
PYLHWYQQKPGKAPKLLIYRVSNRFSGVPSRFSGS
RSGTDFTLTISSLQPEDFATYYCSQGTHVPPTFGQ
GTKVEIK
[10G5 GH1 Heavy chain]
SEQ ID NO. 17
EVQLVQSGAGLVQPGGSVRLSCAASGYSFTDFYIN
WVRQAPGKGLEWIARIFPGGDNTYYNEKFKGRFTL
SADTSSSTAYLQLNSLRAEDTAVYYCARRGLYYAM
DYWGQGTLVTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKKVEPKSCDKTHTCPPCPAPELLGGPSVFLFP
PKPKDTLMISRTPEVTCVVVDVSHEDPEVKFNWYV
DGVEVHNAKTKPREEQYNSTYRVVSVLTVLHQDWL
NGKEYKCKVSNKALPAPIEKTISKAKGQPREPQVY
TLPPSREEMTKNQVSLTCLVKGFYPSDIAVEWESN
GQPENNYKTTPPVLDSDGSFFLYSKLTVDKSRWQQ
GNVFSCSVMHEALHNHYTQKSLSLSPGK
[10G5 GH2 Heavy chain]
SEQ ID NO. 18
EVQLVESGGGLVQPGGSLRLSCAASGYSFTDFYIN
WVRQAPGKGLEWVARIFPGGDNTYYNEKFKGRFTL
SADTSKSTAYLQMNSLRAEDTAVYYCARRGLYYAM
DYWGQGTLVTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKKVEPKSCDKTHTCPPCPAPELLGGPSVFLFP
PKPKDTLMISRTPEVTCVVVDVSHEDPEVKFNWYV
DGVEVHNAKTKPREEQYNSTYRVVSVLTVLHQDWL
NGKEYKCKVSNKALPAPIEKTISKAKGQPREPQVY
TLPPSREEMTKNQVSLTCLVKGFYPSDIAVEWESN
GQPENNYKTTPPVLDSDGSFFLYSKLTVDKSRWQQ
GNVFSCSVMHEALHNHYTQKSLSLSPGK
[10G5 GL1 Light chain]
SEQ ID NO. 19
DIQMTQSPSSLSASVGDRVTITCRSSQSLVHSNGI
PYLHWYQQKPGKAPKLLIYRVSNRFSGVPSRFSGS
GSGTDFTLTISSLQPEDFATYYCSQGTHVPPTFGQ
GTKVEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC
[10G5 GL2 Light chain]
SEQ ID NO. 20
DIQMTQSPSSLSASVGDRVTITCRSSQSLVHSNGI
PYLHWYQQKPGKAPKLLIYRVSNRFSGVPSRFSGS
RSGTDFTLTISSLQPEDFATYYCSQGTHVPPTFGQ
GTKVEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC

### EXAMPLES

The following biological examples are provided by way of illustration, not limitation. In the following biological examples, 1-(6,7-dihydro-5H-benzo[6,7]cyclohepta[1,2-c]pyridazi n-3-yl)-N³-((7-(S)-pyrrolidin-1-yl)-6,7,8,9-tetrahydro-5H-benzo[7]annulene-2-yl)-1H-1,2,4-triazole-3,5-diamine, which is a compound of formula (I), as defined above, is designated in the following examples and the Figures as “BGB324” or Bemcentinib. In the following Examples the immune checkpoint modulator is an anti-PD-1 antibody, as indicated by name in the relevant examples.

STK11/p53/KRAS Mutation Profile is Indicative of Response to AXLi+ICM Combination Therapy in Human NSCLC Patients

Whole Exome Sequence analysis of biopsies collected from responder subjects in a phase II non-small cell lung cancer trial of AXLi (Bemcentinib; BGB324) in combination with ICM (pembrolizumab) identified five subjects with mutations in STK11 and/or STK11IP (STK11 interacting protein). In each case, these responder subjects also had mutations in KRAS and/or p53. Response to the combination therapy was independent of PDL1 status. These findings were unexpected in view of literature reports that subjects with STK11 mutations respond poorly to PD-1 inhibition.

Formalin-fixed paraffin-embedded (FFPE) tumor tissue from 20 patients was subjected to whole exome sequencing, with variants identified using the Pisces 5.2.5.20 (Illumina) variant calling suite (Dunn et al, 2019). The following subjects had identified variants in either STK11 or STK11IP:

| Patient | | | |
|---|---|---|---|
| number | mutations | response | PD-L1 status |
| 234005 | STK11IP, KRAS, P53 | Partial | Negative |
| | | Response (PR) | |
| 234008 | STK11, STK11IP, | PR | Negative |
| | P53, PTEN | | |
| 237001 | STK11IP, KRAS | PR | High |
| 211105 | STK11, P53 | Stable | Weak positive |
| | | Disease (SD) | |
| 211002 | STK11IP, BRAF, P53 | PR | Weak Positive |

A sixth subject (subject number 211003) with a variant STK11 sequence was also identified, however this subject was non-evaluable for response. Predicted severity of the identified mutations was assessed using mutationassessor.org (polyphen-2)—a computational system for predicting the functional impact of protein missense mutations based on phenomenological analysis of information extracted from protein family alignments of large numbers of homologous sequences grouped into aligned sets (families and subfamilies) and the 3D structures of sequence homologs (Reva et al, 2011). The identified mutations and predicted functional impact were as follows:

| | | Predicted |
|---|---|---|
| Patient | | severity/functional |
| number | mutations | impact |
| 211003 | STK11 - L160P | High Probably-damaging |
| 234005 | STK111P - LG334FW | Low + Medium |
| | | Probably-damaging |
| 234008 | STK11 - LD140PY | Medium + Neutral |
| | STK11IP - W162C | Probably-damaging |
| | | Low Probably-damaging |
| 237001 | STK11IP - R1065Q | Medium Probably-damaging |
| 211105 | STKIP - D115V | Medium Probably-damaging |
| 211002 | STK11IP - E19V | Low Probably-damaging |

Patient Results

Patient 234008, a 79-year-old male, initially achieved a partial response to carboplatin and paclitaxel first-line therapy for 22 months before developing progression of lung and adrenal metastases as well as bone marrow metastasis and enrolling in the study. The patient's tumor biopsy was negative for PD-L1 expression but showed AXL expression in both tumor and immune cells. The patient went on to achieve a partial response to the bemcentinib/pembrolizumab combination lasting 11 months from the start of treatment with a maximum target lesion shrinkage of 50.6%. The patient was still alive 2 years after starting treatment.

Patient 211105, a 73-year-old male, was treated with first-line pemetrexed and cisplatin and progressed after 8 months to pembrolizumab monotherapy, where he experienced clinical benefit for 18 months before developing progression of lymph node and chest wall metastases. At the time of screening, the patient's tumor biopsy was PD-L1 weak positive and showed strong expression of AXL in tumor-infiltrating immune cells. The patient experienced clinical benefit from the study drug combination, achieving stable disease for 5.4 months.

Treatment in Patients who Have Previously Received an Anti-PD(L)1 Monoclonal Antibody as Monotherapy or Combination Therapy

Patients were recruited for a phase II trial for the combination treatment of an AXL inhibitor and an anti-PD1/L1 mAb therapy. Patients had previously been treated with a mono therapy PD-L1 or PD-1 inhibitor and had previously demonstrated disease control on this treatment.

The previous therapies include standard dosing regimes such as:
Pembrolizumab (200 mg every 3 weeks, administered over a 30-minute infusion, until disease progression or unacceptable toxicity, or up to 24 months without disease progression;
Pembrolizumab in combination with pemetrexed and platinum chemotherapy (200 mg IV q3Weeks OR 400 mg q6Weeks until disease progression, unacceptable toxicity, or up to 24 months without disease progression);
Atezolizumab (840 mg IV q2Weeks or 1200 mg IV q3Weeks or 1680 mg IV q4Weeks until disease progression or unacceptable toxicity);
Atezolizumab in combination with 1) bevacizumab, paclitaxel, and carboplatin or 2) paclitaxel protein-bound and carboplatin uses the same dosages;
Nivolumab (240 mg IV q2Weeks or 480 mg IV q4Weeks continue until disease progression or unacceptable toxicity)
Nivolumab in combination with Ipilimumab (Nivolumab 3 mg/kg IV q2Weeks plus Ipilimumab 1 mg/kg IV q6Weeks, continue until disease progression, unacceptable toxicity, or up to 24 months without disease progression; or
Nivolumab in combination with Ipilimumab and platinum chemotherapy (Nivolumab 360 mg/kg IV q3Weeks PLUS Ipilimumab 1 mg/kg IV q6Weeks PLUS Histology-based platinum doublet chemotherapy q3Weeks for 2 cycles continue until disease progression, unacceptable toxicity, or up to 24 months without disease progression.

The patients all had progressive disease upon screening. 21 patients were screened and 16 patients were enrolled on the trial. Patient demographics are shown below:

| Patient disposition | Number of Patients |
|---|---|
| Screened | 21 |
| Enrolled | 16 |
| Evaluable* | 15 |
| Ongoing | 3 |

*with at least 1 post-baseline scan assessment.

| Patient demographics | | N (%) |
|---|---|---|
| Age | Median | 64.5 |
| | Range | 40-76 |
| Eastern Cooperative | 0 | 6 (38) |
| Oncology Group | 1 | 10 (63) |
| (ECOG) at screen | | |
| Sex | Female | 3 (19) |
| | Male | 13 (81) |
| Smoking status | Smoker | 6 (38) |
| | Ex-smoker | 8 (50) |
| | Never smoked | 0 (0) |
| | Unknown | 1 (6) |

| Disease Mutations | N (%) |
|---|---|
| None | 13 (81) |
| KRAS | 2 (13) |
| BRAF | 1 (6) |

25% of the patients enrolled on the trial were PD-L1 negative (tumor proportion score (TPS)<1%), 42% PD-L1 positive (TPS 1-49%) and 33% PD-L1 strong positive (TPS>50%).

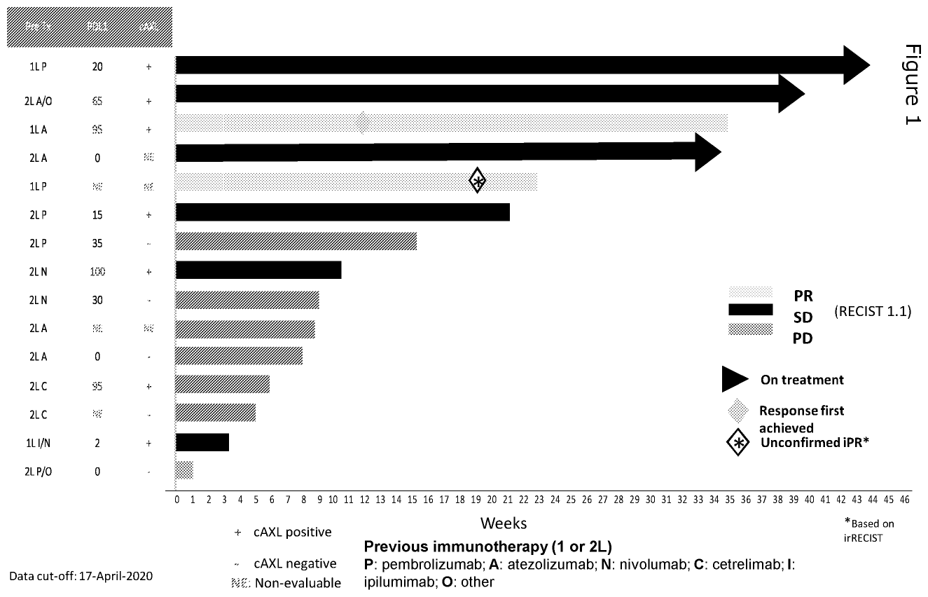

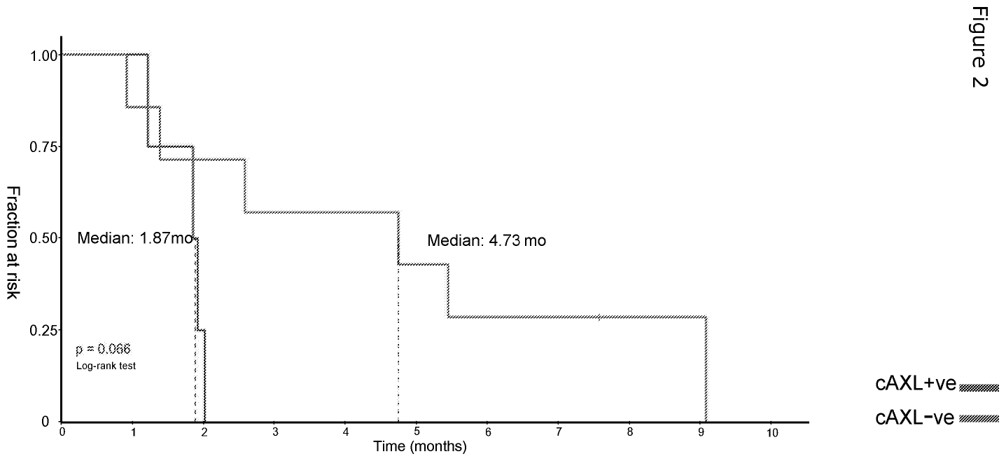

The results are shown in FIG. 1A. Of the 15 patients, 6 showed stable disease and 2 showed a partial response. The results in FIG. 1B demonstrate that 71% of patients in the cAXL positive group showed stable disease and 14% showed a partial response.

FIG. 2 shows the progression free survival (PFS) for the patients. The cAXL positive patients had an mPFS of 4.73 months, cAXL negative patients had an mPFS of 1.87 months.

Results

This demonstrates that a combination of an AXL inhibitor and an Anti-PD1 antibody therapy has surprising results in patients that have previously been treated with a mono immunotherapy but have since suffered disease progression.

STK11 Mutation Abrogates Anti-PD-1 Efficacy

Cell Culture

Kras^{G12D}, p53^{-/-} mutant (KP9-1) or Kras^{G12D}, p53^{-/-} STK11^{-/-} mutant (KPL9-3) mouse lung cancer-derived cell lines were prepared using standard techniques known in the art. KP9-1 or KPL9-3 cells were propagated at sub-confluence and split on a regular basis.

Subcutaneous Tumor Inoculation

Each animal was weighed before cell implantation. Injection of cells was performed after anesthetizing of the mice. The cell lines discussed above were injected subcutaneously into immune-competent mice (C57Bl/6 strain).

Mice were treated with either placebo or anti-PD1 therapeutic (200 μg/mouse, intraperitoneal, twice a week for two weeks).Tumor volume was assessed using hand held callipers and mouse weight were followed.

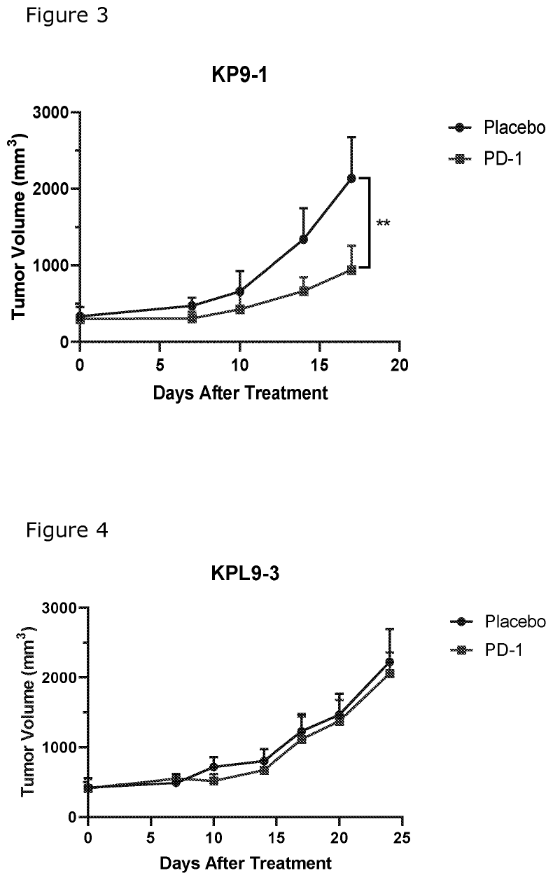

Tumors derived from the Kras^{G12D}, p53^{-/-} STK11^{-/-} mutant cells were resistant to anti-PD1 therapy. Whereas, the tumors derived from the P53 −/− mic were sensitive to anti-PD-1 therapy. The results are shown in FIGS. 3 (P53 −/−) and 4 (STK11 −/−).

Results

These results show that the STK11 mutation reduces the efficacy of anti-PD-1 treatment.

AXL Inhibition Sensitizes STK11 Mutant Cells to Immunotherapy

The Kras^{G12D}, p53^{-/-} STK11^{-/-} mutant (KPL9-3) mouse lung cancer-derived cells described above were implanted subcutaneously in immune-competent mice (C57Bl/6 strain) using the same protocol as described above.

Mice were treated with either placebo, anti-PD1 therapeutic (10 mg/kg, intraperitoneal, twice a week for two weeks), BGB324 (50 mg/kg, oral gavage, twice daily) or a combination of both drugs. Tumor volumes were measured.

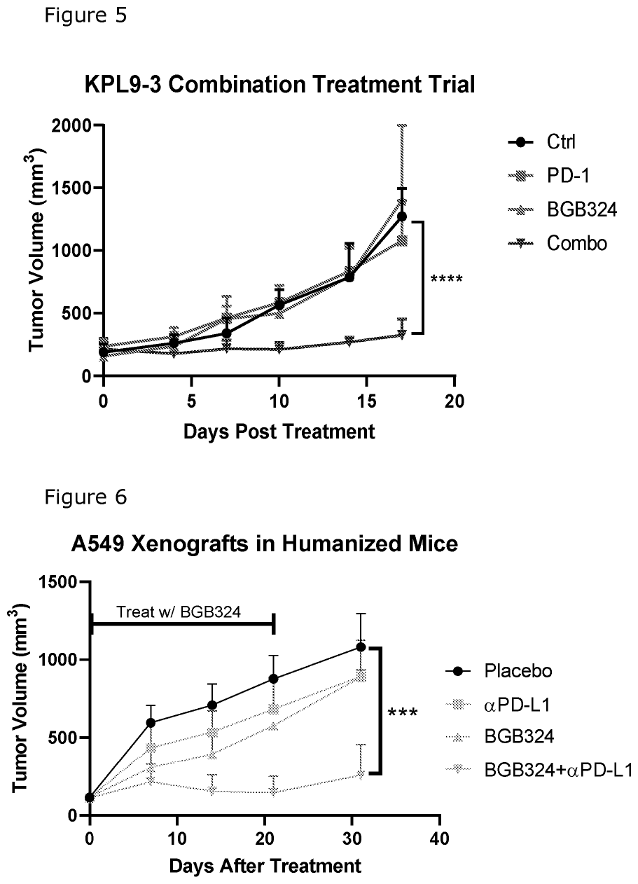

Tumors were resistant to anti-PD1 therapy and BGB324 as monotherapies, but responded to the combination treatment. Tumor volumes are shown in FIG. 5.

Results

These results surprisingly show that an AXL inhibitor is able to sensitise the STK11 mutant cells to treatment with an immune checkpoint inhibitor.

Combination Treatment of an AXL Inhibitor and an Immune Checkpoint Inhibitor in Human Non-Small Cell Lurid Cancer.

Cell Culture

Human non-small cell lung cancer (NSCLC) cells Kras^{G12D}, p53 WT STK11^{-/-} mutant human A549 lung cancer cells were cultured using standard protocols.

Subcutaneous Implantation

Each animal was weighed before cell implantation. Injection of the cells was performed after anesthetizing of the mice. Kras^{G12D}, p53 WT STK11^{-/-} mutant human A549 lung cancer cells were implanted subcutaneously in humanized immune-deficient mice.

Mice were treated with either placebo, anti-PD1 therapy (10 mg/kg, intraperitoneal, 3 dosages), BGB324 (50 mg/kg, oral gavage, twice daily for 3 weeks) or a combination of both drugs.

Tumor volumes were measured and the results are shown in FIG. 6. Once again, tumors were resistant to anti-PD1 therapy and BGB324 as monotherapies, but sensitive to the combination treatment.

Results

These results confirm that an AXL inhibitor is able to sensitise the STK11 mutant cells to treatment with an immune checkpoint inhibitor.

The results in the Examples above demonstrate that treating a subject with a combination of an AXL inhibitor and an immune checkpoint modulator, wherein the subject has modified STK11 activity or expression, will result in a surprisingly good response in this difficult-to-treat patient group.

STK11/LKB1 Mutated NSCLC Lacks Anti-PD-1 Treatment Responsive T Cells in the TME.

Method

For subcutaneous allografts, 1×10⁶ cells in 100 ul phosphate buffered saline (PBS) were injected into the right dorsal flanks of 6-8 wks old mice. For xenografts growing on humanized mice, 1.5×10⁶ cells in 100 ul phosphate buffered saline (PBS) were injected into the right dorsal flanks of reconstituted humanized mice.

Tumor bearing mice were randomly assigned into treatment groups when the tumor grew to around 100-150 mm³. For each treatment group, 5 mice were assigned. 10 mg/kg anti-PD-1 (BioXCell, Cat #BE0146) treatment was given intraperitoneally on day 0, day 4 and day 7 after innoculation. The control group was treated with 10 mg/kg rat IgG2a isotype control (BioX Cell, Cat #BE0089) at the same day of PD-1 treatment, intraperitoneally. For the tumor growth measurement experiment, mice were treated for 3 weeks. For tumor microenvironment (TME) analysis, mice were treated for 7 days, when the tumor sizes in each treatment group are within 2-fold differences. Tumor volumes were measured by length (a), width (b) and height (h) in every 3 days and calculated as tumor volume=abh.

KP9-3 and KPL9-3-1 allografts were harvested 14 days after subcutaneous implantation (average tumor size ˜200 mm³). 10 tumors harvested from each individual mouse within the same group were pooled together as one sample. The CD8⁺ T cell status and composition differences caused by the LKB1 mutation were analysed by aggregating CD8⁺ T cells from KP9-3 and KPL9-3-1 tumors and then clustering into 4 clusters annotated with appropriate markers.

Results

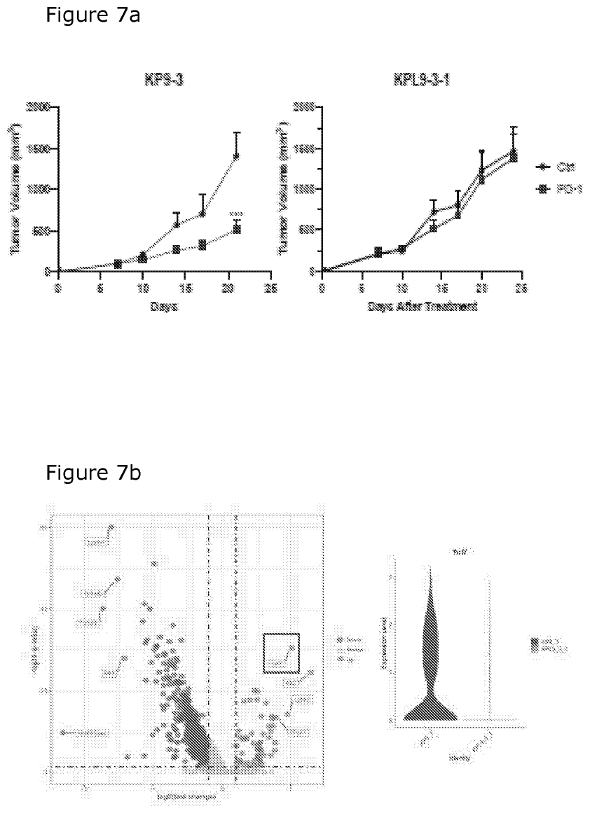

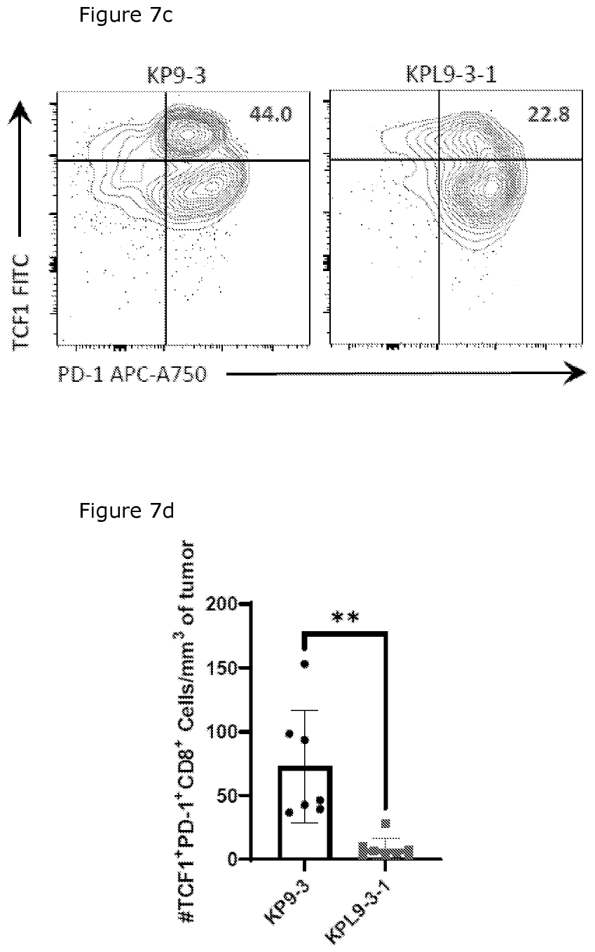

Subcutaneous KP9-3 tumors grew faster than KPL9-3-1 tumors in NSG mice, while KPL9-3-1 grew faster than KP9-3 when injected into C57BL/6J mice. The results in FIG. 7A shows that KPL9-3-1 tumors were poorly responsive to anti-PD-1 therapy while KP9-3 tumors were responsive.

Based on the percentage of CD8⁺ T cells from each cluster, loss of STK11/LKB1 resulted in a more suppressive CD8⁺ T cell composition. In FIG. 7B, a volcano plot is shown (left panel). Points for up- (red) and down- (blue) regulated genes in central memory T cell cluster with Tcf7 are highlighted. All points labelled red or blue lie above the horizontal dashed line, indicating that they are differentially expressed with p<0.05. Blue points lie to the left of the left-hand dashed line, indicating that they are downregulated at least 1.15-fold. Red points lie to the right of the right-hand dashed line, indicating that they are upregulated at least 1.15-fold. The expression level of Tcf7 in CD8+ T cells of KP9-3 and KPL9-3-1 are compared and visualized through violin plot (FIG. 7B, right panel; KP9-3 to left, KPL9-3-1 to right). The results in FIG. 7B show that KP9-3 tumors showed enrichment for central memory T cells with TCF1/7 expression compared to KPL9-3-1 tumors. This result suggests a lack of TCF1 expressing CD8⁺ T cells in tumors that lose STK11/LKB1.

The results were confirmed by comparing TCF1⁺PD-1⁺ CD8⁺ T cells in KP9-3 and KPL9-3-1 tumors through flow cytometry and IHC. The results are shown in FIGS. 7C and D.

Bemcentinib Mediated Axl Inhibition Sensitizes LKB1 Mutant Tumors to Anti-PD-1 Therapy.

Methods

KPL9-3-1 allograft were implanted into mice as described above. 5 mice were assigned into each treatment group. 10 mg/kg anti-PD-1 (BioXCell, Cat #BE0146) treatment was given intraperitoneally on day 0, day 4 and day 7 after treatment. Control group of PD-1 treatment was treated with 10 mg/kg rat IgG2a isotype control (BioX Cell, Cat #BE0089) at the same day of PD-1 treatment, intraperitoneally. BGB324 were given through oral gavage twice daily, with a dose of 50 mg/kg.

For tumor growth measurement experiment, mice were treated for 3 weeks. For tumor microenvironment analysis, mice were treated for 7 days, when the tumor sizes in each treatment group are within 2-fold differences.Seven days after tumor inoculation, mice were treated with either BGB324 or anti-PD-1 alone, or in combination.

The immune landscape of the tumors from each treatment group was assessed using scRNAseq of immune cells. Sequenced scRNA-seq samples were processed through Cellranger pipelines (v3.1.0). Cellranger count was used to align reads to mouse reference genome (mm10, 2020-A, from 10× Genomics) and generate single cell feature counts for single library.

For scTCR-seq data, TCR reads were aligned to reference genome and TCR annotation was performed using the 10×cellranger vdj pipeline with provided reference (cellranger-vdj-GRCm38-alts-ensemb1-4.0.0). Overall, 94% of T cells in scRNA-seq data were assigned a TCR and more than 70% had at least one full-length productive CDR3 for both TRA and TRB. The clonotype of each T cell was represented by the beta CDR3 sequences, and clone sizes ranged from 1 cell to 580 cells. Cellranger aggr were applied next to aggregate each sample library for grouped analysis with same effective sequencing depth. Seurat (3.2.1) package was used for downstream analysis.

To determine the potential lineage differentiation between those T cell populations with high TCR sharing. Monocle (version 2.0) (PMID: 24658644) was used to investigate transcriptional and developmental trajectories concerning different CD8+ or CD4+ T cell clusters. The data of raw counts together with cluster annotations were taken as input to monocle, and the default parameters were set to run data normalization and dimension reduction. Next, Monocle leaned the kinetics of gene expression by using the reversed graph embedding approach and places each cell along an inferred pseudotime trajectory. According to the assumption that the trajectory has a tree structure, functional “State” is identified based on the segment of the tree-like structure.

Results

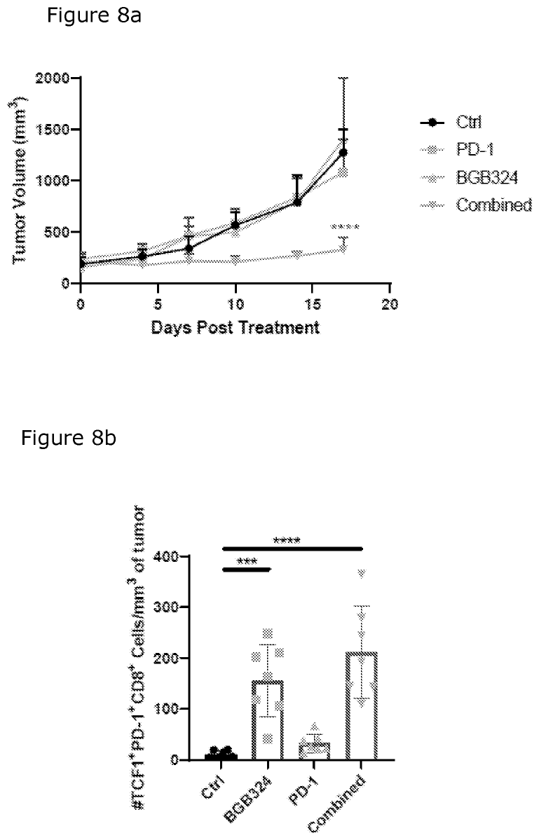

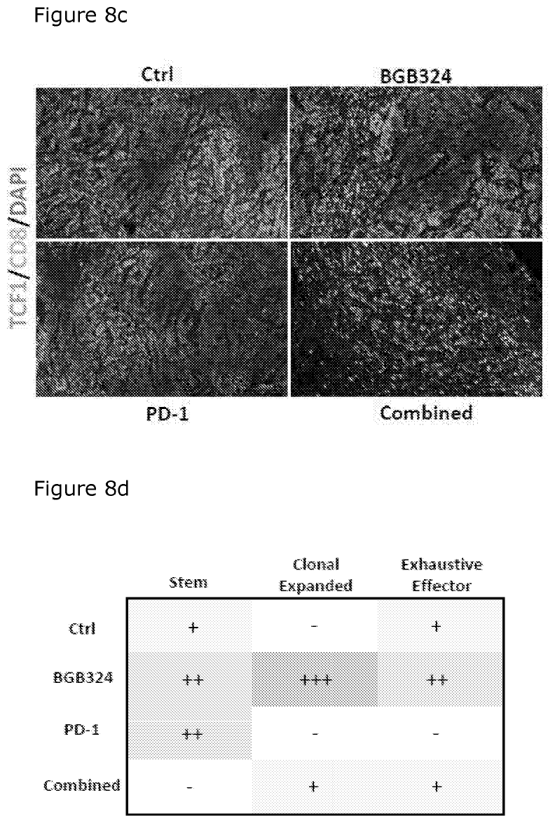

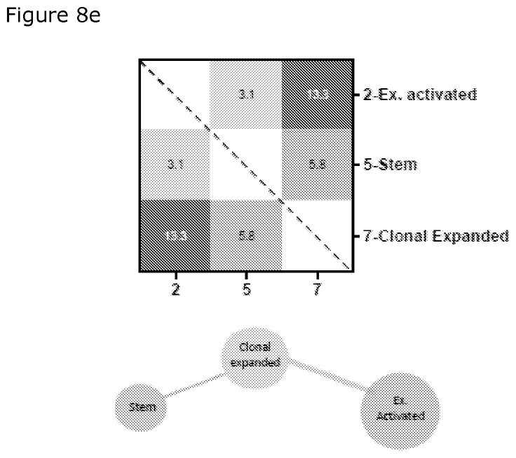

Neither single treatment resulted in tumor growth control. However, the combination of BGB324 with anti-PD-1 treatment showed a synergistic effect with sustained control of tumor progression (FIG. 8A).

BGB324 treatment alone or in combination with anti-PD-1 increased the infiltration of TCF1+PD-1⁺ CD8⁺ T cells (FIG. 8B).

Immunohistochemistry (IHC) analysis demonstrated that while Axl inhibition alone enhanced TCF1⁺PD-1⁺ CD8⁺ T cell presence in the tumor, combination with anti-PD-1 was required to break the exclusion of TCF1⁺CD8⁺ T cells from tumor islands (FIG. 8C). In the left hand panels of FIG. 8C, very few CD8+ cells are seen. Addition of BGB324 results in a large population of CD8+ cells, but these are grouped together, excluded from the tumor islands. Following combination treatment, CD8+ cells are present dispersed throughout the tumor.

To further dissect the dynamics changes in the KPL9-3-1 TME after treatment, scRNAseq with paired TCR sequencing were performed for four pooled samples from each treatment group annotated with representative markers. Sub-clustering of CD8⁺ T cells revealed eight defined annotated sub-populations.

Treatment enriched cells in each cluster were calculated and compared based on the number of cells observed divided by expected number of cells in each cluster (FIG. 8D).

BGB324 treated tumors enriched CD8⁺ T cells expressing unique TCRs (clonal expanded) significantly, with the stem like T cells and exhaustive effector T cells also enriched. Combination therapy showed a trend of enrichment of clonal expanded and exhaustive effector CD8⁺ T cells.

This analysis demonstrates that stem-like T cells correlate the most with clonal expanded T cells, which develop into proliferating and exhaustive effector T cells capable of performing direct tumor cell kill (FIG. 8E).

Bemcentinib-Induced Type I Interferon Secretion Expands TCF1⁺PD-1⁺CD8⁺ T Cells

Methods

Bone marrow derived BMDCs (2×10⁵) were co-cultured with 40 g irradiated KPL9-3-1 tumor cells (2×10⁶), with or without BGB324 treatment (40 nM). After 24 h, the cell supernatant was collected for analysis. The concentration of IFN8 was measured by VeriKine-HS Mouse IFN Beta Serum ELISA kit (PBL Assay Science, Cat #42410), or VerKine Human IFN Beta ELISA kit (PBL Assay Science, Cat #414101) in accordance with the manufacturer's instructions. The plates were visualized by adding 100 μL of TMB solution and read at 450 nm using the SPECTROstarNano (BMG LABTECH).

The cells were treated with BGB324 or DMSO.

To determine the contribution of type I interferon secretion to BGB324 sensitization of KPL9-3-1 tumors to anti-PD-1 treatment the IFNα receptor was inhibitred pharmacologically.

Results

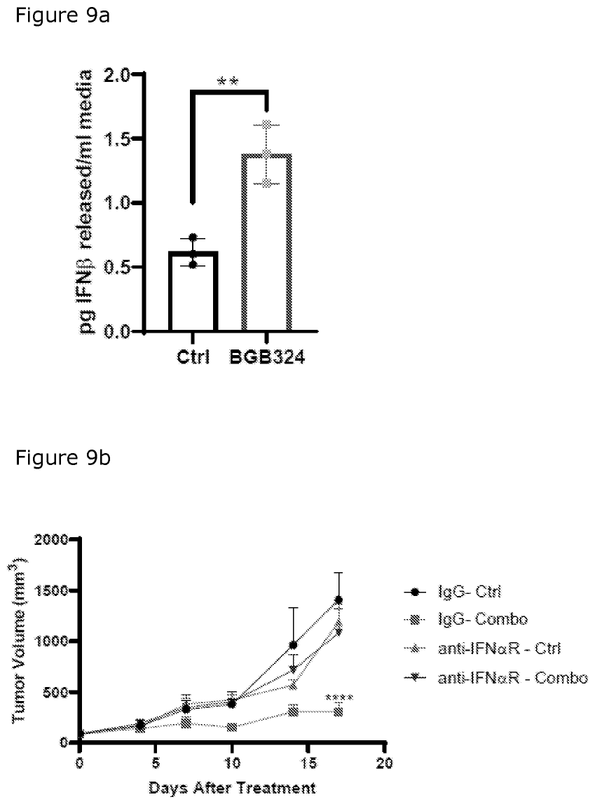

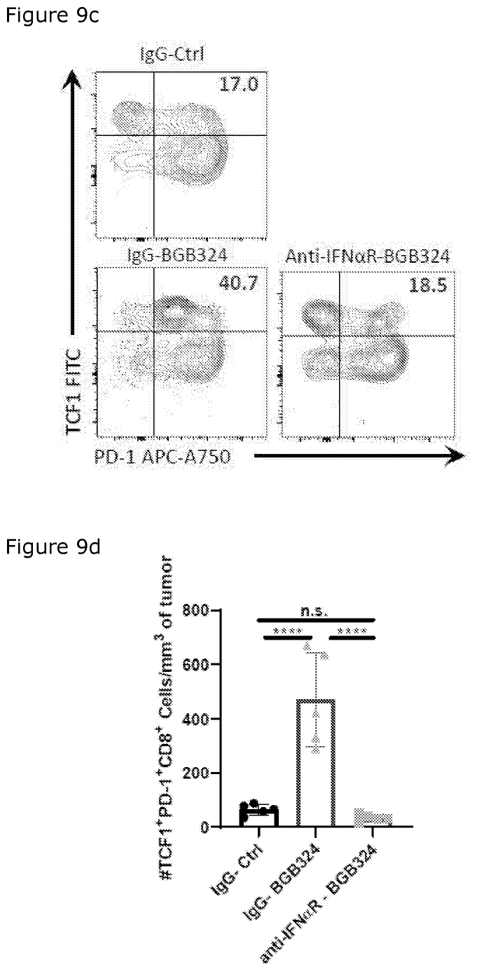

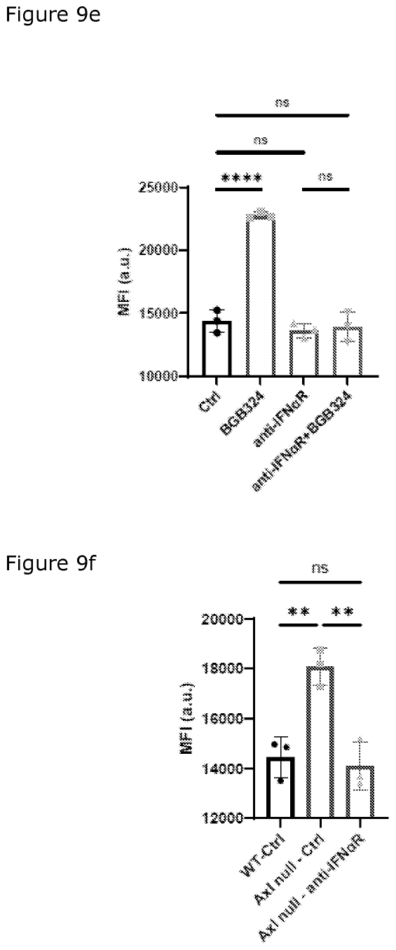

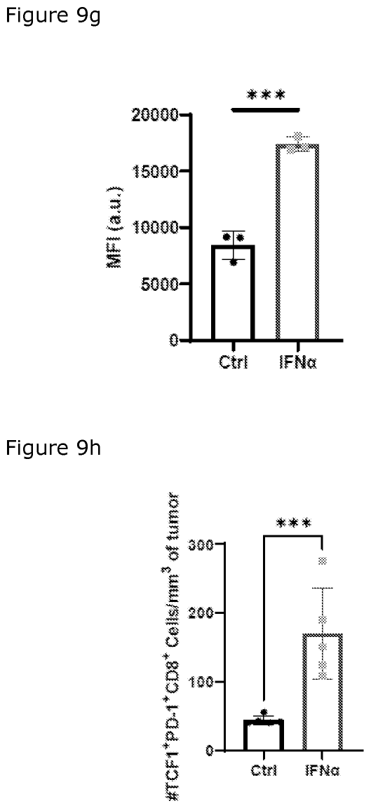

BMDCs treated with BGB324 showed increased secretion of Interferon type 1β (IFNβ) (FIG. 9A). Increased secretion of IFNβ was also observed through co-culturing Axl-deficient BMDCs with irradiated tumor cells, as well as in tumor lysates.

Blocking IFNα receptor intratumorally abrogated the efficacy of combined Axl and PD-1 inhibition (FIG. 9B).

IFNα receptor blockade diminished the infiltration of TCF1⁺PD-1⁺CD8⁺ T cells in the TME (FIGS. 9C-D). Further blocking the IFNα receptor counteracted BGB324 induced TCF1⁺ expression on CD8⁺ T cells in co-cultures of BMDC with OT-1 CD8⁺ T cells stimulated by ovalbumin, suggesting that increased TCF1⁺ expression on CD8⁺ T cells by BGB324 treatment is Type I interferon IFNαR axis dependent (FIG. 9E).

Bemcentinib-Induced Type I Interferon Secretion Expands TCF1⁺PD-1⁺CD8⁺ T Cells in Axl-Deficient BMDCs

Methods

To demonstrate the importance of Type I IFN response, the level of TCF1 expression in OT-1 CD8 T cells stimulated with OVA and cultured with BMDCs +/− IFNα was determined by flow cytometry. Tumor tissues were excised and digested with 2 mg/mL Collagenase A (Sigma, Cat #SCR136) and 1 mg/ml DNase I (Roche, Cat #11284932001) under 37° C., 150 rpm shaking speed for 45 min. Digested materials then were transferred to a 70 μm cell strainer to remove clumped cells. Digested cells were washed twice with FACs buffer and ready for flow cytometry analysis.

Results

The results are shown in FIG. 9F. Stimulation with IFNα increased TCF1 expression on T cells (FIG. 9G). These results were phenocopied by intratumoral injection of IFNα (FIG. 9H).

Conclusion

Taken together, these results demonstrate that increased IFN I secretion as a result of Axl inhibition is critical for inducing TCF1⁺ PD-1⁺CD8⁺ T cell expansion in KPL9-3-1 tumors to overcome anti-PD-1/PD-L1 resistance.

Mutation of KRAS and STK11 Results in High AXL in Tumor Immune Cells

Methods

Tissue microarray analysis of 62 NSCLC patients was performed. 7 patients had mutation of both KRAS and STK11. 55 were KL wild type.

Tissue microarrays (TMAs) were generated with surgical resected non-small cell lung carcinoma tumor samples (TMA3). IHC staining was performed on 4-μm thick TMA sections in a Leica Bond RX automated strainer (Leica Biosystems). The antigen retrieval was performed with Bond ER Solution #1 (Leica Biosystems) equivalent to citrate buffer, pH 9.0 for 20 min at 100° C. The sections were then incubated with anti-AXL antibody (rabbit monoclonal, Cell Signaling clone C89E7); 1:300 dilution (1 μg/ml). The antibody was detected using a Bond Polymer Refine Detection kit (Leica Biosystems) with diaminobenzidine (DAB) as the chromogen. All the slides were counterstained with hematoxylin, dehydrated, and cover slipped. Tonsil and normal colon sections were used as external positive controls. Each case was analyzed using standard microscopy by two pathologists in immune cells and reported as percentage of the tumor area occupied with immune cell with cytoplasmic and/or membrane expression.

Results

All the KL mutant patients had high AXL in tumor immune cells; whereas, in KL wild type patients, only 20 of the 55 patients displayed high AXL.

Conclusion

These results demonstrate that KRAS and STK11 mutations are good predictors of AXL activity. Patients with KRAS and/or STK11 mutations will benefit from treatment with a combination of an AXL inhibitor (AXLi) and an immune checkpoint modulator (ICM).

Statements of Disclosure

The following numbered statements, outlining aspects of the present disclosure, are part of the description.

Methods of Selecting a Subject for Treatment

101. A method of selecting a subject for treatment with a combination of an AXL inhibitor (AXLi) and an immune checkpoint modulator (ICM), the method comprising: identifying subjects having an AXL-related disease characterised by the presence of cells having modified STK11 activity or expression; and, selecting thus identified subjects for treatment.

102. A method of selecting a subject for treatment with a combination of an AXL inhibitor (AXLi) and an immune checkpoint modulator (ICM), the method comprising: identifying subjects having an AXL-related disease characterised by the presence of cells having increased KRAS activity or expression; and, selecting thus identified subjects for treatment.

103. A method of selecting a subject for treatment with a combination of an AXL inhibitor (AXLi) and an immune checkpoint modulator (ICM), the method comprising: identifying subjects that have previously been treated with an immune checkpoint modulator (ICM) and which did not respond to treatment with the ICM; and, selecting thus identified subjects for treatment.

104. The method of statement 103, wherein the method further comprises: identifying subjects having an AXL-related disease characterised by the presence of cells having modified STK11 activity or expression; and, selecting thus identified subjects for treatment.

105a. A method of selecting a subject for treatment with a combination of an AXL inhibitor (AXLi), an immune checkpoint modulator (ICM), and a chemotherapeutic agent and/or radiotherapy, the method comprising: identifying subjects having an AXL-related disease characterised by the presence of cells having modified STK11 activity or expression; and, selecting thus identified subjects for treatment.

105b. A method of selecting a subject for treatment with a combination of an AXL inhibitor (AXLi), an immune checkpoint modulator (ICM), and a chemotherapeutic agent and/or radiotherapy, the method comprising: identifying subjects having an AXL-related disease characterised by the presence of cells having increased KRAS activity or expression; and, selecting thus identified subjects for treatment.

106. The method of statement 105a or statement 105b, wherein the method further comprises: identifying subjects that have previously been treated with a combination of an immune checkpoint modulator (ICM) and chemotherapeutic agent and/or radiotherapy, wherein treatment with the combination of immune checkpoint modulator (ICM) and chemotherapeutic agent and/or radiotherapy did not provide any additional benefit as compared to treatment with the chemotherapeutic agent and/or radiotherapy alone; and, selecting thus identified subjects for treatment.

107. A method of selecting a subject for treatment with a combination of an AXL inhibitor (AXLi), an immune checkpoint modulator (ICM), and a chemotherapeutic agent and/or radiotherapy, the method comprising: identifying subjects that have previously been treated with a combination of an immune checkpoint modulator (ICM) and chemotherapeutic agent and/or radiotherapy, wherein treatment with the combination of immune checkpoint modulator (ICM) and chemotherapeutic agent and/or radiotherapy did not provide any additional benefit as compared to treatment with the chemotherapeutic agent and/or radiotherapy alone; and, selecting thus identified subjects for treatment.

108. The method of statement 107, wherein the method further comprises: identifying subjects having an AXL-related disease characterised by the presence of cells having decreased STK11 activity or expression; and, selecting thus identified subjects for treatment.

Patient Sub-Group Definition

109. The method of any preceding statement, wherein the method comprises: identifying subjects wherein the AXL-related disease is further characterised by the presence of cells having increased KRAS activity or expression; and, selecting thus identified subjects for treatment.

110. The method of any preceding statement, wherein the method comprises: identifying subjects wherein the AXL-related disease is further characterised by the presence of cells having decreased p53 activity or expression; and, selecting thus identified subjects for treatment.

111. The method of any preceding statement, wherein the method comprises: identifying subjects wherein the AXL-related disease is further characterised by increased activity or expression of AXL as compared to a control.

Assessing Increased/Decreased Expression

112. The method of any preceding statement, wherein increased or decreased expression is assessed by determining copy number of the gene encoding STK11, KRAS, or p53 relative to a control sample, wherein an increase in the copy number indicates an increased level of expression and a decrease in the copy number indicates a decreased level of expression.

113. The method of any preceding statement, wherein increased or decreased expression is assessed by determining the level of STK11, KRAS, or p53 protein or mRNA relative to a control sample.

114. The method of any preceding statement, wherein modified STK11 activity or expression is assessed by determining the presence or absence of a STK11 mutation and/or a STK11IP mutation.

115. The method of any one of statements 110-114, wherein increased KRAS activity or expression is assessed by determining the presence of absence of a KRAS mutation.

116. The method of any one of statements 111-115, wherein decreased p53 activity or expression is assessed by determining the presence of absence of a p53 mutation.

117. The method of any one of statements 114-116, wherein the STK11 mutation, STK11IP mutation, KRAS mutation, and/or p53 mutation is a mutation selected from:
(i) a mutation in the nucleotide sequence encoding STK11, STK11IP, KRAS, or p53;
(ii) a mutation in a regulatory sequence controlling expression of the nucleotide sequence encoding STK11, STK11IP, KRAS, or p53;
(iii) a mutation in a nucleotide encoding a protein which interacts with the transcription product of the STK11, STK11IP, KRAS, or p53 gene.

118. The method of any one of statements 114-117, wherein the STK11 mutation, STK11IP mutation, KRAS mutation, and/or p53 mutation is a mutation in the translation product of the STK11, STK11IP, KRAS, or p53 gene.

119. The method of any one of statements 114-118, wherein the STK11 mutation, STK11IP mutation, KRAS mutation, and/or p53 mutation is a mutation in the transcription product of the STK11, STK11IP, KRAS, or p53 gene.

120. The method of any one of statements 114-119, wherein the STK11 mutation is an inactivating mutation.

121. The method of any one of statements 114-120, wherein the STK11IP mutation is an activating mutation.

122. The method of any one of statements 115-121, wherein the KRAS mutation is an activating mutation.

123. The method of statement 122, wherein the KRAS mutation is a mutation at position G12, optionally wherein the KRAS mutation is a G12D mutation.

124. The method of any one of statements 116-123, wherein the p53 mutation is an inactivating mutation.

125. The method of any preceding statement, wherein increased or decreased activity or expression is determined in a sample derived from a subject.

126. The method of any preceding statement, wherein increased or decreased activity or expression is determined relative to a control.

127. The method of statement 126, wherein the control is healthy tissue, preferably of the same tissue type as the AXL-related disease.

AXL-Related Disease

128. The method of any preceding statement, wherein the AXL-related disease is a proliferative disease.

129. The method of any preceding statement, wherein the AXL-related disease is a neoplastic disease.

130. The method of any preceding statement, wherein the AXL-related disease is a solid tumour.

131. The method of any preceding statement, wherein the AXL-related disease is cancer.

132. The method of statement 131, wherein the cancer is selected from the group consisting of: lung cancer, non-small-cell lung cancer, breast cancer, melanoma, mesothelioma, acute myeloid leukemia (AML), myelodysplatic syndrome (MDS), pancreas cancer, kidney cancer, urothelial carcinoma, and glioblastoma.

133. The method of statement 131, wherein the cancer is lung cancer.

134. The method of statement 131, wherein the cancer is non-small-cell lung cancer (NSCLC).

135. The method of any preceding statement, wherein the AXL-related disease does not respond to or benefit from treatment with an immune checkpoint modulator (ICM) when administered alone or as part of a treatment regime that does not include an AXLi.

136. The method of any preceding statement, wherein the AXL-related disease is characterised by cells having a STK11 mutation and/or a STK11IP mutation.

137. The method of any preceding statement, wherein the AXL-related disease is characterised by cells having a KRAS mutation.

138. The method of any preceding statement, wherein the AXL-related disease is characterised by the presence of cells having a p53 mutation.

139. The method of any preceding statement, wherein the AXL-related disease is characterised by the presence of cells having a STK11 mutation, a KRAS mutation, and a p53 mutation.

140. The method of any preceding statement, wherein the AXL-related disease is characterised by the presence of cells having a STK11IP mutation, a KRAS mutation, and a p53 mutation.

141. The method of any preceding statement, wherein the AXL-related disease is not an AXL-related disease characterised by increased KRAS activity or expression, and wild-type STK11 and/or p53 activity or expression.

142. The method of any preceding statement, wherein the AXL-related disease is not an AXL-related disease characterised by: the presence of cells having a KRAS mutation; and, the absence of cells having a STK11, STK11IP, and/or p53 mutation.

143. The method of any preceding statement, wherein the AXL-related disease is not an AXL-related disease characterised by the presence of cells having a KRAS G12C mutation.

144. The method of any preceding statement, wherein the AXL-related disease is not an AXL-related disease characterised by cells which do not have a STK11, STK11IP, and/or p53 mutation.

145. The method of any preceding statement, wherein the AXL-related disease is not a Lewis Lung Carcinoma (LLC) or Lewis Lung model tumour.

Treatment Step

146. The method of any preceding statement, further comprising treating the subject in a method of treatment according to any one of statements 201-285.

147. The method according to any one of statements 101-145, further comprising administering to the subject a therapeutically effective amount of an AXL inhibitor (AXLi), an immune checkpoint modulator (ICM), and/or a chemotherapeutic agent and/or radiotherapy.

148. The method of statement 147, wherein the AXL inhibitor is administered concurrently with the immune checkpoint modulator (ICM).

149. The method of statement 147, wherein the AXL inhibitor is administered concurrently with the immune checkpoint modulator (ICM) and/or the chemotherapeutic agent and/or radiotherapy.

150. The method of statement 147, wherein the AXL inhibitor is administered separately and/or sequentially to the immune checkpoint modulator (ICM).

151. The method of statement 147, wherein the AXL inhibitor is administered separately and/or sequentially to the immune checkpoint modulator (ICM) and/or the chemotherapeutic agent and/or radiotherapy.

152. The method of statement 147, wherein the AXL inhibitor is administered subsequent to administration of the immune checkpoint modulator (ICM).

153. The method of statement 147, wherein the AXL inhibitor is administered subsequent to administration of the immune checkpoint modulator (ICM) and/or subsequent to administration of the chemotherapeutic agent and/or radiotherapy.

154. The method of statement 147, wherein the immune checkpoint modulator (ICM) is administered subsequent to administration of the Axl inhibitor.

155. The method of statement 147, wherein the immune checkpoint modulator (ICM) is administered subsequent to administration of the Axl inhibitor and/or subsequent to administration of the chemotherapeutic agent and/or radiotherapy.

156. The method of statement 147, wherein the chemotherapeutic agent and/or radiotherapy is administered subsequent to administration of the AXL inhibitor and/or subsequent to administration of the immune checkpoint modulator (ICM).

157. The method of statement 147, wherein:
i) the chemotherapeutic agent and/or radiotherapy is administered subsequent to administration of the AXL inhibitor; and
ii) the immune checkpoint modulator (ICM) is administered subsequent to administration of the chemotherapeutic agent and/or radiotherapy.

158. The method of statement 147, wherein the method comprises:
i) administering the AXL inhibitor to the subject, wherein the immune checkpoint modulator (ICM) has been, is, or will be, administered to the subject; and/or
ii) administering the AXL inhibitor to the subject, wherein the chemotherapeutic agent and/or radiotherapy has been, is, or will be, administered to the subject.

159. The method of statement 147, wherein the method comprises:
i) administering the immune checkpoint modulator (ICM) to the subject, wherein the AXL inhibitor has been, is, or will be, administered to the subject; and/or
ii) administering the immune checkpoint modulator (ICM) to the subject, wherein the chemotherapeutic agent and/or radiotherapy has been, is, or will be, administered to the subject.

160. The method of statement 147, wherein the method comprises:
i) administering the chemotherapeutic agent and/or radiotherapy to the subject, wherein the AXL inhibitor has been, is, or will be, administered to the subject; and/or
ii) administering the chemotherapeutic agent and/or radiotherapy to the subject, wherein the immune checkpoint modulator (ICM) has been, is, or will be, administered to the subject.

AXLi

161. The method of any preceding statement, wherein the AXL inhibitor is a compound of formula (I) as set out in the description.

162. The method of statement 161, wherein the AXL inhibitor is selected from the group consisting of:
1-(6,7-dihydro-5H-benzo[6,7]cyclohepta[1,2-c]pyridazin-3-yl)-N3-(7-(pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7-(S)-pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7-(R)-pyrrolidin-1-yl)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-(3-fluoro-4-(4-(pyrrolidin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N5-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N5-(7-(S)-pyrrolidin-1-yl-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(acetamido)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-((2R)-2-(methoxycarbonyl)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(4,4-difluoropiperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-((methoxycarbonylmethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-((2R)-2-(carboxy)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(4-(ethoxycarbonyl)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(4-(carboxy)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-((carboxymethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(4-(ethoxycarbonylmethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(4-(carboxymethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N3-((7S)-7-amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7s)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((2-methylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((propyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(dipropylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(diethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(cyclopentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((1-cyclopentylethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(2-propylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((3,3-dimethylbut-2-yl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(di(cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((5-chlorothien-2-yl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((2-carboxyphenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((3-bromophenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(dimethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(3-pentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((2,2-dimethylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(di(cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(di(bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(3-methylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(2-ethylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(but-2-enylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(butyl(but-2-enyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N5-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-amino-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(dimethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(diethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(dipropylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-((methylethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(cyclopentylamino)-6,7,8,9-tetrahydro-5H-benzo[7]annulene-2-yl)-1H-1,2,4-triazole-3,5-diamine; and
1-(6,7-dihydro-5H-pyrido[2′,3′:6,7]cyclohepta[1,2-c]pyridazin-3-yl)-N3-((7S)-7-(2-butylamino)-6,7,8,9-tetrahydro-5H-benzo[7]annulene-2-yl)-1H-1,2,4-triazole-3,5-diamine;
or pharmaceutically acceptable salts thereof.

163. The method of statement 161, wherein the AXL inhibitor is 1-(6,7-dihydro-5H-benzo[6,7]cyclohepta[1,2-c]pyridazin-3-yl)-N3-((7-(S)-pyrrolidin-1-yl)-6,7,8,9-tetrahydro-5H-benzo[7]annulene-2-yl)-1H-1,2,4-triazole-3,5-diamine, or a pharmaceutically acceptable salt thereof.

164. The method of statement 161, wherein the AXL inhibitor is Bemcentinib (BGB324/R428).

165. The method of any one of statements 101-160, wherein the AXL inhibitor is selected from the group consisting of: dubermatinib (CAS No.1341200-45-0; UNII 14D65TV20J); gilteritinib (CAS No. 1254053-43-4; UNII 66D92MGC8M); cabozantinib (CAS No. 849217-68-1; UNII 1C39JW444G); SG17079 (CAS No. 1239875-86-5); merestinib (CAS No. 1206799-15-6; UNII 50GS5K699E); amuvatinib (CAS No. 850879-09-3; UNII SO9S6QZB4R); bosutinib (CAS No. 380843-75-4; UNII 5018V4AEZ0); glesatinib (CAS No. 936694-12-1; UNII 7Q290XD98N); foretinib (CAS No. 849217-64-7; UNII 81 FH7VK1 C4); and, TP0903 (CAS No. 1341200-45-0).

166. The method of any one of statements 101-160, wherein the AXL inhibitor is an AXL inhibitor disclosed in WO2008/083367, WO2010/083465, or WO2012/028332.

167. The method of any one of statements 101-160, wherein the AXL inhibitor is an anti-AXL antibody.

168. The method of statement 167, wherein the antibody is an anti-AXL antibody disclosed in WO2015/193428, WO2015/193430, WO2016/097370, or WO2016/166296.

169. The method of statement 167, wherein the antibody is an anti-AXL antibody selected from the group consisting of: the 1613F12 antibody disclosed in WO2013/064685; the 110D7 antibody disclosed in WO2014/068139; the 1003A2 antibody disclosed in WO2014/068139; the 1024G11 antibody disclosed in WO2014/068139; the hu10G5 antibody disclosed in WO2017/220695; and, the YW327.6S2 antibody disclosed in WO2011/159980.

170. The method of statement 167, wherein the antibody comprises the 6 CDRs having the sequences of SEQ ID Nos. 1 to 6.

171. The method of statement 167, wherein the antibody comprises the 6 CDRs having the sequences of SEQ ID Nos. 7 to 12.

172. The method of statement 167, wherein the antibody comprises:
a VH domain having the sequence of SEQ ID No. 13 and a VL domain having the sequence of SEQ ID NO. 15;
a VH domain having the sequence of SEQ ID No. 13 and a VL domain having the sequence of SEQ ID NO. 16;
a VH domain having the sequence of SEQ ID No. 14 and a VL domain having the sequence of SEQ ID NO. 15; or
a VH domain having the sequence of SEQ ID No. 14 and a VL domain having the sequence of SEQ ID NO. 16.

ICM

173. The method of any preceding statement, wherein the immune checkpoint modulator includes one or more immune checkpoint inhibitors (101).

174. The method of any preceding statement, wherein the immune checkpoint modulator includes one or more immune checkpoint modulating antibody.

175. The method of statement 174, wherein one or more immune checkpoint modulating antibody is selected from the group consisting of: anti-CTLA-4 antibodies, anti-PD-1 antibodies, anti-PD-L1 antibodies, anti-4-1 BB antibodies, anti-OX-40 antibodies, anti-GITR antibodies, anti-CD27 antibodies, anti-CD28 antibodies, anti-CD40 antibodies, anti-LAGS antibodies, anti-ICOS antibodies, anti-TWEAKR antibodies, anti-HVEM antibodies, anti-TIM-1 antibodies, anti-TIM-3 antibodies, anti-VISTA antibodies, and anti-TIGIT antibodies.

176. The method of statement 174, wherein one or more immune checkpoint modulating antibody is selected from the group consisting of: anti-CTLA-4 antibodies, anti-PD-1 antibodies, anti-PD-L1 antibodies, anti-4-1 BB antibodies, anti-OX-40 antibodies, anti-GITR antibodies, anti-CD27 antibodies, anti-CD40 antibodies, and anti-LAGS antibodies.

177. The method of statement 174, wherein one or more immune checkpoint modulating antibody is selected from the group consisting of: anti-CTLA-4 antibodies, anti-PD-1 antibodies, and anti-PD-L1 antibodies.

178. The method of any preceding statement, wherein the immune checkpoint modulator includes: one or more T-cell co-stimulatory agonist; and/or one or more dendritic cell co-stimulatory receptor agonist.

179. The method of any preceding statement, wherein the immune checkpoint modulator includes at least two immune checkpoint modulators.

180. The method of any preceding statement, wherein the immune checkpoint modulator includes: (i) an immune checkpoint inhibitor, and (ii) a T cell co-stimulatory receptor agonist or a dendritic cell co-stimulatory receptor agonist.

181. The method of any preceding statement, wherein the immune checkpoint modulator includes: (i) an anti-CTLA-4 antibody; and, (ii) an anti-PD-1 antibody and/or an anti-PD-L1 antibody.

182. The method of statement 181, wherein the anti-CTLA-4 antibody is ipilimumab or tremelimumab.

183. The method of statement 181, wherein the anti-PD-1 antibody is pembrolizumab or nivolumab.

184. The method of statement 181, wherein the anti-PD-L1 antibody is atezolizumab (CAS number 1380723-44-3), avelumab (CAS number 1537032-82-8), or durvalumab (CAS number 1428935-60-7).

185. The method of any preceding statement, wherein the immune checkpoint modulator includes, or is: pembrolizumab; ipilimumab; ipilimumab and nivolumab; ipilimumab and pembrolizumab; tremelilumab and durvalumab.

186. The method of any one of statements 179 to 185, wherein the at least two immune checkpoint modulators are administered concurrently.

187. The method of any one of statements 179 to 185, wherein the at least two immune checkpoint modulators are administered separately and/or sequentially.

Chemotherapeutic Agent/Radiotherapy

188. The method of any one of statements 101-104 and 109-187, wherein the treatment comprises treatment with a combination of an AXL inhibitor (AXLi), an immune checkpoint modulator (ICM), and a chemotherapeutic agent and/or radiotherapy.

189. The method of any one of statements 105-188, wherein the chemotherapeutic agent is a chemotherapeutic agent which induces immunogenic cell death of cancer cells.

190. The method of any one of statements 105-188, wherein the chemotherapeutic agent is a chemotherapeutic agent which induces an immune response in the subject.

191. The method of any one of statements 105-188, wherein the chemotherapeutic agent is a chemotherapeutic agent which induces a type I interferon response in the subject.

192. The method of any one of statements 105-188, wherein the chemotherapeutic agent is an anthracycline.

193. The method of statement 192, wherein the anthracycline is doxorubicin, daunorubicin, epirubicin, idarubicin, mitoxantrone, or valrubicin.

194. The method of statement 192, wherein the anthracycline is doxorubicin.

Methods of Treatment

201a. A method of treating an AXL-related disease in a subject with a combination of an AXL inhibitor (AXLi) and an immune checkpoint modulator (ICM), the method comprising:
(i) administering a combination of the AXLi and the ICM to the subject;
(ii) administering the AXLi to the subject, wherein the ICM has been, is, or will be, administered to the subject; or
(iii) administering the ICM to the subject, wherein the AXLi has been, is, or will be, administered to the subject;
wherein the AXL-related disease is characterised by the presence of cells having modified STK11 activity or expression.

201b. A method of treating an AXL-related disease in a subject with a combination of an AXL inhibitor (AXLi) and an immune checkpoint modulator (ICM), the method comprising:
(i) administering a combination of the AXLi and the ICM to the subject;
(ii) administering the AXLi to the subject, wherein the ICM has been, is, or will be, administered to the subject; or
(iii) administering the ICM to the subject, wherein the AXLi has been, is, or will be, administered to the subject;
wherein the AXL-related disease is characterised by the presence of cells having increased KRAS activity or expression.

202a. A method of treating an AXL-related disease in a subject with a combination of an AXL inhibitor (AXLi) and an immune checkpoint modulator (ICM), the method comprising:
(i) administering a combination of the AXLi and the ICM to the subject;
(ii) administering the AXLi to the subject, wherein the ICM has been, is, or will be, administered to the subject; or
(iii) administering the ICM to the subject, wherein the AXLi has been, is, or will be, administered to the subject;
wherein the AXL-related disease is characterised by the presence of cells having a STK11 mutation or a STK11IP mutation.

202b. A method of treating an AXL-related disease in a subject with a combination of an AXL inhibitor (AXLi) and an immune checkpoint modulator (ICM), the method comprising:
(i) administering a combination of the AXLi and the ICM to the subject;
(ii) administering the AXLi to the subject, wherein the ICM has been, is, or will be, administered to the subject; or
(iii) administering the ICM to the subject, wherein the AXLi has been, is, or will be, administered to the subject;
wherein the AXL-related disease is characterised by the presence of cells having a KRAS mutation.

203a. A method of treating an AXL-related disease in a subject with a combination of an AXL inhibitor (AXLi) and an immune checkpoint modulator (ICM), the method comprising:
(i) administering a combination of the AXLi and the ICM to the subject;
(ii) administering the AXLi to the subject, wherein the ICM has been, is, or will be, administered to the subject; or
(iii) administering the ICM to the subject, wherein the AXLi has been, is, or will be, administered to the subject;
wherein the subject has been selected for treatment on the basis that the AXL-related disease is characterised by the presence of cells having decreased STK11 activity or expression.

203b. A method of treating an AXL-related disease in a subject with a combination of an AXL inhibitor (AXLi) and an immune checkpoint modulator (ICM), the method comprising:
(i) administering a combination of the AXLi and the ICM to the subject;
(ii) administering the AXLi to the subject, wherein the ICM has been, is, or will be, administered to the subject; or
(iii) administering the ICM to the subject, wherein the AXLi has been, is, or will be, administered to the subject;
wherein the subject has been selected for treatment on the basis that the AXL-related disease is characterised by the presence of cells having increased KRAS activity or expression.

204. A method of treating an AXL-related disease in a subject with a combination of an AXL inhibitor (AXLi) and an immune checkpoint modulator (ICM), the method comprising:
(i) administering a combination of the AXLi and the ICM to the subject;
(ii) administering the AXLi to the subject, wherein the ICM has been, is, or will be, administered to the subject; or
(iii) administering the ICM to the subject, wherein the AXLi has been, is, or will be, administered to the subject;
wherein the subject has been selected for treatment using a method according to any one of statements 101-194.

### Patient Sub-Group Definition

202. The method of any preceding statement, wherein the AXL-related disease is further characterised by the presence of cells having increased KRAS activity or expression.

203. The method of any preceding statement, wherein the AXL-related disease is further characterised by the presence of cells having decreased p53 activity or expression.

204. The method of any preceding statement, wherein the AXL-related disease is further characterised by increased activity or expression of AXL.

205. The method of any preceding statement, wherein the AXL-related disease is further characterised by the presence of cells having a KRAS mutation.

206. The method of any preceding statement, wherein the AXL-related disease is further characterised by the presence of cells having a p53 mutation.

207. The method of any preceding statement, wherein the AXL-related disease is characterised by the presence of cells having a STK11 mutation, a KRAS mutation, and a p53 mutation.

208. The method of any preceding statement, wherein the AXL-related disease is characterised by the presence of cells having a STK11IP mutation, a KRAS mutation, and a p53 mutation.

209. The method of any preceding statement, wherein the STK11 mutation, STK11IP mutation, KRAS mutation, and/or p53 mutation is a mutation selected from:
(i) a mutation in the nucleotide sequence encoding STK11, STK11IP, KRAS, or p53;
(ii) a mutation in a regulatory sequence controlling expression of the nucleotide sequence encoding STK11, STK11IP, KRAS, or p53; or
(iii) a mutation in a nucleotide encoding a protein which interacts with the transcription product of the STK11, STK11IP, KRAS, or p53 gene.

210. The method of any preceding statement, wherein the STK11 mutation, STK11IP mutation, KRAS mutation, and/or p53 mutation is a mutation in the translation product of the STK11, STK11IP, KRAS, or p53 gene.

211. The method of any preceding statement, wherein the STK11 mutation, STK11IP mutation, KRAS mutation, and/or p53 mutation is a mutation in the transcription product of the STK11, STK11IP, KRAS, or p53 gene.

212. The method of any preceding statement, wherein the STK11 mutation is an inactivating mutation.

213. The method of any preceding statement, wherein the STK11IP mutation is an activating mutation.

214. The method of any preceding statement, wherein the KRAS mutation is an activating mutation.

215. The method of any preceding statement, wherein the p53 mutation is an inactivating mutation.

216. The method of any preceding statement, wherein the STK11 mutation results in a reduced level of activity or expression of STK11 protein.

217. The method of any preceding statement, wherein the STK11IP mutation results in an increased level of activity or expression of STK11IP protein.

218. The method of any preceding statement, wherein the STK11IP mutation results in an altered pattern of activity or expression of STK11 protein, and/or altered subcellular localisation of STK11 protein.

219. The method of any preceding statement, wherein the KRAS mutation results in an increased level of activity or expression of KRAS protein.

220. The method of statement 219, wherein the KRAS mutation is a mutation at position G12, optionally wherein the KRAS mutation is a G12D mutation.

221. The method of any preceding statement, wherein the p53 mutation results in a reduced level of activity or expression of p53 protein.

222. The method of any preceding statement, wherein increased or decreased activity or expression is determined in a sample derived from a subject.

223. The method of any preceding statement, wherein increased or decreased activity or expression is determined relative to a control.

224. The method of statement 224, wherein the control is healthy tissue, preferably of the same tissue type as the AXL-related disease.

AXL-Related Disease

225. The method of any preceding statement, wherein the AXL-related disease is a proliferative disease.

226. The method of any preceding statement, wherein the AXL-related disease is a neoplastic disease.

227. The method of any preceding statement, wherein the AXL-related disease is a solid tumour.

228. The method of any preceding statement, wherein the AXL-related disease is cancer.

229. The method of statement 228, wherein the cancer is selected from the group consisting of: lung cancer, non-small-cell lung cancer, breast cancer, melanoma, mesothelioma, acute myeloid leukemia (AML), myelodysplatic syndrome (MDS), pancreas cancer, kidney cancer, urothelial carcinoma, and glioblastoma.

230. The method of statement 228, wherein the cancer is lung cancer.

231. The method of statement 228, wherein the cancer is non-small-cell lung cancer (NSCLC). 232. The method of any preceding statement, wherein the AXL-related disease is not an AXL-related disease characterised by increased KRAS activity or expression, and wild-type STK11 and/or p53 activity or expression.

233. The method of any preceding statement, wherein the AXL-related disease is not an AXL-related disease characterised by: the presence of cells having a KRAS mutation; and, the absence of cells having a STK11, STK11IP, and/or p53 mutation.

234. The method of any preceding statement, wherein the AXL-related disease is not an AXL-related disease characterised by the presence of cells having a KRAS G12C mutation.

235. The method of any preceding statement, wherein the AXL-related disease is not an AXL-related disease characterised by cells which do not have a STK11, STK11IP, and/or p53 mutation.

236. The method of any preceding statement, wherein the AXL-related disease is not a Lewis Lung Carcinoma (LLC) or Lewis Lung model tumour.

237. The method of any preceding statement, wherein the AXL-related disease does not respond to or benefit from treatment with an immune checkpoint modulator (ICM) when administered alone or as part of a treatment regime that does not include an AXLi.

AXLi

238. The method of any preceding statement, wherein the AXL inhibitor is a compound of formula (I) as set out in the description.

239. The method of statement 238, wherein the AXL inhibitor is selected from the group consisting of:
1-(6,7-dihydro-5H-benzo[6,7]cyclohepta[1,2-c]pyridazin-3-yl)-N3-(7-(pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7-(S)-pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7-(R)-pyrrolidin-1-yl)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-(3-fluoro-4-(4-(pyrrolidin-1-yl)piperidin-1-yl)phenyl)-1H-1,2,4-triazole-3,5-diamine;

1-(6,7-dihydro-5H-benzo[6,7]cyclohepta[1,2-c]pyridazin-3-yl)-N5-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N5-(7-(S)-pyrrolidin-1-yl-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(acetamido)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-((2R)-2-(methoxycarbonyl)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(4,4-difluoropiperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-((methoxycarbonylmethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-((2R)-2-(carboxy)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(4-(ethoxycarbonyl)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(4-(carboxy)pipendin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-((carboxymethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(4-(ethoxycarbonylmethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(4-(carboxymethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N3-((7S)-7-amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7s)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((2-methylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((propyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(dipropylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(diethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(cyclopentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((1-cyclopentylethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(2-propylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((3,3-dimethylbut-2-Aamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(di(cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((5-chlorothien-2-yl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((2-carboxyphenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((3-bromophenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(dimethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(3-pentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((2,2-dimethylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(di(cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(di(bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)((bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(3-methylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(2-ethylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(but-2-enylamino)-6,7,8,9-tetrahydro-5H-benzo[7]annulene-2-yl)-1H-1,2,4-triazole-3,5-diamine;

1-(6,7-dihydro-5H-benzo[6,7]cyclohepta[1,2-c]pyridazin-3-yl)-N3-((7S)-7-(butyl(but-2-enyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N5-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-amino-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(dimethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(diethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(dipropylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-((methylethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(cyclopentylamino)-6,7,8,9-tetrahydro-5H-benzo[7]annulene-2-yl)-1H-1,2,4-triazole-3,5-diamine; and
1-(6,7-dihydro-5H-pyrido[2′,3′:6,7]cyclohepta[1,2-c]pyridazin-3-yl)-N3-((7S)-7-(2-butylamino)-6,7,8,9-tetrahydro-5H-benzo[7]annulene-2-yl)-1H-1,2,4-triazole-3,5-diamine;
or pharmaceutically acceptable salts thereof.

240. The method of statement 238, wherein the AXL inhibitor is 1-(6,7-dihydro-5H-benzo[6,7]cyclohepta[1,2-c]pyridazin-3-yl)-N3-((7-(S)-pyrrolidin-1-yl)-6,7,8,9-tetrahydro-5H-benzo[7]annulene-2-yl)-1H-1,2,4-triazole-3,5-diamine, or a pharmaceutically acceptable salt thereof.

241. The method of statement 238, wherein the AXL inhibitor is Bemcentinib (BGB324/R428).

242. The method of any one of statements 201-237, wherein the AXL inhibitor is selected from the group consisting of: dubermatinib (CAS No. 1341200-45-0; UNII 14D65TV20J); gilteritinib (CAS No. 1254053-43-4; UNII 66D92MGC8M); cabozantinib (CAS No. 849217-68-1; UNII 1C39JW444G); SG17079 (CAS No. 1239875-86-5); merestinib (CAS No. 1206799-15-6; UNII 50GS5K699E); amuvatinib (CAS No. 850879-09-3; UNII SO9S6QZB4R); bosutinib (CAS No. 380843-75-4; UNII 5018V4AEZ0); glesatinib (CAS No. 936694-12-1; UNII 7Q290XD98N); foretinib (CAS No. 849217-64-7; UNII 81 FH7VK1 C4); and, TP0903 (CAS No. 1341200-45-0).

243. The method of any one of statements 201-237, wherein the AXL inhibitor is an AXL inhibitor disclosed in WO2008/083367, WO2010/083465, or WO2012/028332.

244. The method of any one of statements 201-237, wherein the AXL inhibitor is an anti-AXL antibody.

245. The method of statement 244, wherein the antibody is an anti-AXL antibody disclosed in WO2015/193428, WO2015/193430, WO2016/097370, or WO2016/166296.

246. The method of statement 244, wherein the antibody is an anti-AXL antibody selected from the group consisting of: the 1613F12 antibody disclosed in W02013/064685; the 110D7 antibody disclosed in WO2014/068139; the 1003A2 antibody disclosed in WO2014/068139; the 1024G11 antibody disclosed in WO2014/068139; the hu10G5 antibody disclosed in WO2017/220695; and, the YW327.6S2 antibody disclosed in WO2011/159980.

247. The method of statement 244, wherein the antibody comprises the 6 CDRs having the sequences of SEQ ID Nos. 1 to 6.

248. The method of statement 244, wherein the antibody comprises the 6 CDRs having the sequences of SEQ ID Nos. 7 to 12.

249. The method of statement 244, wherein the antibody comprises:
a VH domain having the sequence of SEQ ID No. 13 and a VL domain having the sequence of SEQ ID NO.15;
a VH domain having the sequence of SEQ ID No. 13 and a VL domain having the sequence of SEQ ID NO.16;
a VH domain having the sequence of SEQ ID No. 14 and a VL domain having the sequence of SEQ ID NO.15; or
a VH domain having the sequence of SEQ ID No. 14 and a VL domain having the sequence of SEQ ID NO.16.

ICM

250. The method of any preceding statement, wherein the immune checkpoint modulator includes one or more immune checkpoint inhibitors (101).

251. The method of any preceding statement, wherein the immune checkpoint modulator includes one or more immune checkpoint modulating antibody.

252. The method of statement 251, wherein one or more immune checkpoint modulating antibody is selected from the group consisting of: anti-CTLA-4 antibodies, anti-PD-1 antibodies, anti-PD-L1 antibodies, anti-4-1 BB antibodies, anti-OX-40 antibodies, anti-GITR antibodies, anti-CD27 antibodies, anti-CD28 antibodies, anti-CD40 antibodies, anti-LAGS antibodies, anti-ICOS antibodies, anti-TWEAKR antibodies, anti-HVEM antibodies, anti-TIM-1 antibodies, anti-TIM-3 antibodies, anti-VISTA antibodies, and anti-TIGIT antibodies.

253. The method of statement 251, wherein one or more immune checkpoint modulating antibody is selected from the group consisting of: anti-CTLA-4 antibodies, anti-PD-1 antibodies, anti-PD-L1 antibodies, anti-4-1 BB antibodies, anti-OX-40 antibodies, anti-GITR antibodies, anti-CD27 antibodies, anti-CD40 antibodies, and anti-LAGS antibodies.

254. The method of statement 251, wherein one or more immune checkpoint modulating antibody is selected from the group consisting of: anti-CTLA-4 antibodies, anti-PD-1 antibodies, and anti-PD-L1 antibodies.

255. The method of any preceding statement, wherein the immune checkpoint modulator includes: one or more T-cell co-stimulatory agonist; and/or one or more dendritic cell co-stimulatory receptor agonist.

256. The method of any preceding statement, wherein the immune checkpoint modulator includes at least two immune checkpoint modulators.

257. The method of any preceding statement, wherein the immune checkpoint modulator includes: (i) an immune checkpoint inhibitor, and (ii) a T cell co-stimulatory receptor agonist or a dendritic cell co-stimulatory receptor agonist.

258. The method of any preceding statement, wherein the immune checkpoint modulator includes: (i) an anti-CTLA-4 antibody; and, (ii) an anti-PD-1 antibody and/or an anti-PD-L1 antibody.

259. The method of statement 258, wherein the anti-CTLA-4 antibody is ipilimumab or tremelimumab.

260. The method of statement 258, wherein the anti-PD-1 antibody is pembrolizumab or nivolumab.

261. The method of statement 258, wherein the anti-PD-L1 antibody is atezolizumab (CAS number 1380723-44-3), avelumab (CAS number 1537032-82-8), or durvalumab (CAS number 1428935-60-7).

262. The method of any preceding statement, wherein the immune checkpoint modulator includes, or is: pembrolizumab; ipilimumab; ipilimumab and nivolumab; ipilimumab and pembrolizumab; tremelilumab and durvalumab.

263. The method of any one of statements 256 to 262, wherein the at least two immune checkpoint modulators are administered concurrently.

264. The method of any one of statements 256 to 262, wherein the at least two immune checkpoint modulators are administered separately and/or sequentially.

Triple Combination with Chemotherapeutic Agent/Radiotherapy

265. The method of any preceding statement, wherein the subject is treated with a combination of an AXL inhibitor (AXLi), an immune checkpoint modulator (ICM), and a chemotherapeutic agent and/or radiotherapy.

266. The method of statement 265, wherein the chemotherapeutic agent is a chemotherapeutic agent which induces immunogenic cell death of cancer cells.

267. The method of any one of statements 265-266, wherein the chemotherapeutic agent is a chemotherapeutic agent which induces an immune response in the subject.

268. The method of any one of statements 265-267, wherein the chemotherapeutic agent is a chemotherapeutic agent which induces a type I interferon response in the subject.

269. The method of any one of statements 265-268, wherein the chemotherapeutic agent is an anthracycline.

270. The method of statement 269, wherein the anthracycline is doxorubicin, daunorubicin, epirubicin, idarubicin, mitoxantrone, or valrubicin.

271. The method of statement 269, wherein the anthracycline is doxorubicin.

272. The method of any one of statements 265-271, wherein treatment of the AXL-related disease with a combination of immune checkpoint modulator (ICM) and chemotherapeutic agent does not provide any additional benefit as compared to treatment with the chemotherapeutic agent alone.

Administration Schedule Features

273. The method of any one of statements 201-272, wherein the AXL inhibitor is administered concurrently with the immune checkpoint modulator (ICM).

274. The method of any one of statements 265-272, wherein the AXL inhibitor is administered concurrently with the immune checkpoint modulator (ICM) and/or the chemotherapeutic agent and/or radiotherapy.

275. The method of any one of statements 201-272, wherein the AXL inhibitor is administered separately and/or sequentially to the immune checkpoint modulator (ICM).

276. The method of any one of statements 265-272, wherein the AXL inhibitor is administered separately and/or sequentially to the immune checkpoint modulator (ICM) and/or the chemotherapeutic agent and/or radiotherapy.

277. The method of any one of statements 201-272, wherein the AXL inhibitor is administered subsequent to administration of the immune checkpoint modulator (ICM).

278. The method of any one of statements 265-272, wherein the AXL inhibitor is administered subsequent to administration of the immune checkpoint modulator (ICM) and/or subsequent to administration of the chemotherapeutic agent and/or radiotherapy.

279. The method of any one of statements 201-272, wherein the immune checkpoint modulator (ICM) is administered subsequent to administration of the Axl inhibitor.

280. The method of any one of statements 265-272, wherein the immune checkpoint modulator (ICM) is administered subsequent to administration of the Axl inhibitor and/or subsequent to administration of the chemotherapeutic agent and/or radiotherapy.

281. The method of any one of statements 265-272, wherein the chemotherapeutic agent and/or radiotherapy is administered subsequent to administration of the AXL inhibitor and/or subsequent to administration of the immune checkpoint modulator (ICM).

282. The method of any one of statements 265-272, wherein:
the chemotherapeutic agent and/or radiotherapy is administered subsequent to administration of the AXL inhibitor; and
the immune checkpoint modulator (ICM) is administered subsequent to administration of the chemotherapeutic agent and/or radiotherapy.

283. The method of any one of statements 265-272, wherein the method comprises:
i) administering the AXL inhibitor to the subject, wherein the immune checkpoint modulator (ICM) has been, is, or will be, administered to the subject; and/or
ii) administering the AXL inhibitor to the subject, wherein the chemotherapeutic agent and/or radiotherapy has been, is, or will be, administered to the subject.

284. The method of any one of statements 265-272, wherein the method comprises:
i) administering the immune checkpoint modulator (ICM) to the subject, wherein the AXL inhibitor has been, is, or will be, administered to the subject; and/or
ii) administering the immune checkpoint modulator (ICM) to the subject, wherein the chemotherapeutic agent and/or radiotherapy has been, is, or will be, administered to the subject.

285. The method of any one of statements 265-272, wherein the method comprises:
i) administering the chemotherapeutic agent and/or radiotherapy to the subject, wherein the AXL inhibitor has been, is, or will be, administered to the subject; and/or
ii) administering the chemotherapeutic agent and/or radiotherapy to the subject, wherein the immune checkpoint modulator (ICM) has been, is, or will be, administered to the subject.

Second Medical Uses

301. An AXL inhibitor for use in a method of treating an AXL-related disease according to any one of statements 201-285.

302. An immune checkpoint modulator (ICM) for use in a method of treating an AXL-related disease according to any one of statements 201-285.

303. An AXL inhibitor and an immune checkpoint modulator (ICM) for use in a method of treating an AXL-related disease according to any one of statements 201-285.

304. A chemotherapeutic agent for use in a method of treating an AXL-related disease according to any one of statements 265-285.

305. An AXL inhibitor, an immune checkpoint modulator (1CM), and a chemotherapeutic agent for use in a method of treating an AXL-related disease according to any one of statements 265-285.

306. A reagent for detecting activity, expression, or amount of STK11, STK11IP, KRAS, or p53, for use in a method of selecting a subject for treatment with a combination of an AXL inhibitor (AXLi) and an immune checkpoint modulator (1CM).

307. A kit comprising 1, 2, 3, 4, or more reagents for detecting activity, expression, or amount of one or more of STK11, STK11IP, KRAS, or p53, for use in a method of selecting a subject for treatment with a combination of an AXL inhibitor (AXLi) and an immune checkpoint modulator (ICM).

308. The reagent or kit for use according to statement 306 or 307, wherein the method of selecting is a method according to any one of statements 101-194.

309. The reagent or kit for use according to any one of statements 306-308, wherein each reagent for detecting is a specific binding member which is selective for STK11, STK11IP, KRAS, or p53.

310. The reagent or kit for use according to any one of statements 306-308, wherein the reagent for detecting is an antibody, a nucleic acid probe, or a QPCR primer.

Swiss Format

401. Use of an AXL inhibitor in the manufacture of a medicament for treating a disorder in a subject, wherein the treatment comprises a method of treating an AXL-related disease according to any one of statements 201-285.

402. Use of an immune checkpoint modulator (ICM) in the manufacture of a medicament for treating a disorder in a subject, wherein the treatment comprises a method of treating an AXL-related disease according to any one of statements 201-285.

403. Use of an AXL inhibitor and an immune checkpoint modulator (ICM) in the manufacture of a medicament for treating a disorder in a subject, wherein the treatment comprises a method of treating an AXL-related disease according to any one of statements 201-285.

404. Use of a chemotherapeutic agent in the manufacture of a medicament for treating a disorder in a subject, wherein the treatment comprises a method of treating an AXL-related disease according to any one of statements 265-285.

405. Use of an AXL inhibitor, an immune checkpoint modulator (ICM), and a chemotherapeutic agent in the manufacture of a medicament for treating a disorder in a subject, wherein the treatment comprises a method of treating an AXL-related disease according to any one of statements 265-285.

406. Use of a reagent for detecting activity, expression, or amount of STK11, STK11IP, KRAS, or p53, in the manufacture of a kit or test for use in a method of selecting a subject for treatment with a combination of an AXL inhibitor (AXLi) and an immune checkpoint modulator (ICM).

407. Use of 1, 2, 3, 4, or more reagents for detecting activity, expression, or amount of one or more of STK11, STK11IP, KRAS, or p53, in the manufacture of a kit for use in a method of selecting a subject for treatment with a combination of an AXL inhibitor (AXLi) and an immune checkpoint modulator (ICM).

408. The use according to statement 406 or 407, wherein the method of selecting is a method according to any one of statements 101-194.

409. The use according to any one of statements 406-408, wherein each reagent for detecting is a specific binding member which is selective for STK11, STK11IP, KRAS, or p53.

410. The use according to any one of statements 406-408, wherein the reagent for detecting is an antibody, a nucleic acid probe, or a QPCR primer.

Kits

501. A kit comprising an AXL inhibitor and an immune checkpoint modulator (ICM), for use in a method of treating an Axl-related disease according to any one of statements 201-285.

502. A kit comprising an AXL inhibitor, an immune checkpoint modulator (ICM), and a chemotherapeutic agent, for use in a method of treating an Axl-related disease according to any one of statements 265-285.

503. A kit comprising a chemotherapeutic agent and an AXL inhibitor and/or an immune checkpoint modulator (ICM), for use in a method of treating an Axl-related disease according to any one of statements 265-285.

Prognostic Methods

601a. A method of prognosing susceptibility of a subject to treatment with a combination of an AXL inhibitor (AXLi) and an immune checkpoint modulator (ICM), the method comprising:
determining: i) the presence or absence of a STK11 mutation; and/or ii) the level of STK11 activity or expression in the subject or a sample derived from the subject;
wherein the presence of a STK11 mutation and/or a modified level of STK11 activity or expression is indicative of susceptibility to treatment with a combination of an AXL inhibitor (AXLi) and an immune checkpoint modulator (ICM).

601b. A method of prognosing susceptibility of a subject to treatment with a combination of an AXL inhibitor (AXLi) and an immune checkpoint modulator (ICM), the method comprising:
determining: i) the presence or absence of a KRAS mutation; and/or ii) the level of KRAS activity or expression in the subject or a sample derived from the subject;
wherein the presence of a KRAS mutation and/or an increased level of KRAS activity or expression is indicative of susceptibility to treatment with a combination of an AXL inhibitor (AXLi) and an immune checkpoint modulator (ICM).

602a. A method of prognosing susceptibility of a subject to treatment in a method according to any one of statements 201-285, the method comprising:
determining: i) the presence or absence of a STK11 mutation; and/or ii) the level of STK11 activity or expression in the subject or a sample derived from the subject;
wherein the presence of a STK11 mutation and/or a modified level of STK11 activity or expression is indicative of susceptibility to treatment with a combination of an AXL inhibitor (AXLi) and an immune checkpoint modulator (ICM).

602b. A method of prognosing susceptibility of a subject to treatment in a method according to any one of statements 201-285, the method comprising:
determining: i) the presence or absence of a KRAS mutation; and/or ii) the level of KRAS activity or expression in the subject or a sample derived from the subject;
wherein the presence of a KRAS mutation and/or an increased level of KRAS activity or expression is indicative of susceptibility to treatment with a combination of an AXL inhibitor (AXLi) and an immune checkpoint modulator (ICM).

603. The method according to any preceding statement, wherein the method further comprises:
determining: i) the presence or absence of a KRAS mutation; and/or ii) the level of KRAS activity or expression in the subject or a sample derived from the subject;
wherein the presence of a KRAS mutation and/or an increased level of KRAS activity or expression is indicative of susceptibility to treatment with a combination of an AXL inhibitor (AXLi) and an immune checkpoint modulator (ICM).

604. The method of any preceding statement, wherein the method further comprises:
determining: i) the presence or absence of a p53 mutation; and/or ii) the level of p53 activity or expression in the subject or a sample derived from the subject;
wherein the presence of a p53 mutation and/or an decreased level of p53 activity or expression is indicative of susceptibility to treatment with a combination of an AXL inhibitor (AXLi) and an immune checkpoint modulator (ICM).

605. The method of any preceding statement, wherein the method comprises:
determining the level of AXL activity or expression in the subject or a sample derived from the subject;
wherein an increased level of AXL activity or expression is indicative of susceptibility to treatment with a combination of an AXL inhibitor (AXLi) and an immune checkpoint modulator (ICM).

606. A method of prognosing susceptibility of a subject to treatment with a combination of an AXL inhibitor (AXLi) and an immune checkpoint modulator (ICM), the method comprising testing subjects in a method according to any one of statements 101-194.

607. A method of prognosing susceptibility of a subject to treatment in a method according to any one of statements 201-285, the method comprising testing subjects in a method according to any one of statements 101-194.

608. The method of any one of statements 601-607, further comprising treating a subject determined to be susceptible to treatment in a method according to any one of statements 201-285.

700. A method of selecting a subject for treatment with a combination of an AXL inhibitor (AXLi) and an immune checkpoint modulator (ICM), the method comprising: identifying subjects having an AXL-related disease characterised by a reduced presence of CD8⁺ cells having TCF1 activity or expression; and, selecting thus identified subjects for treatment.

701. The method of statement 700, wherein the method further comprises: identifying subjects that have previously been treated with an immune checkpoint modulator (ICM) and which did not respond to treatment with the ICM; and, selecting thus identified subjects for treatment.

702. The method of statement 700 or 701, further comprising the method of any one of statements 109-194.

800. A method of increasing a population of desired T cells in a subject comprising treating the subject with an AXL inhibitor (AXLi).

801. The method of statement 800, wherein the desired T cell population is a CD8+ T cell population.

802. The method of 800 or 801, wherein the desired T cell population is a TCF1⁺PD-1⁺CD8⁺ T cell population.

803. The method of any one of statements 800 to 802, wherein the subject is further treated with an immune checkpoint inhibitor as described in any one of statements 250-264.

### REFERENCES

A number of publications are cited above in order to more fully describe and disclose the invention and the state of the art to which the invention pertains. Full citations for these references are provided below. The entirety of each of these references is incorporated herein.
Rothlin C V, Ghosh S, Zuniga E I, Oldstone M B, Lemke G. TAM receptors are pleiotropic inhibitors of the innate immune response. Cell. 2007; 131(6):1124-36.
Morén A et al, Negative regulation of TGFβ signaling by the kinase LKB1 and the scaffolding protein LIP1., J Biol Chem. 2011 Jan. 7; 286(1):341-53
Huang M T, Liu W L, Lu C W, Huang J J, Chuang H L, Huang Y T, et al. Feedback regulation of IFN-alpha/beta signaling by Axl receptor tyrosine kinase modulates HBV immunity. Eur J Immunol. 2015; 45(6)1 696-705.
Chen J, Yang YF, Yang Y, Zou P, Chen J, He Y, et al. AXL promotes Zika virus infection in astrocytes by antagonizing type I interferon signalling. Nature microbiology. 2018; 3(3):302-9.
Galluzzi L, Buque A, Kepp O, Zitvogel L, Kroemer G. Immunological Effects of Conventional Chemotherapy and Targeted Anticancer Agents. Cancer cell. 2015; 28(6):690-714.
Yan Y, Kumar A B, Finnes H, Markovic S N, Park S, Dronca R S, et al. Combining Immune Checkpoint Inhibitors With Conventional Cancer Therapy. Front Immunol. 2018; 9:1739.
Emens L A, Adams S, Loi S, Schneeweiss A, Rugo H S, Winer E P, et al. IMpassion130: a Phase III randomized trial of atezolizumab with nab-paclitaxel for first-line treatment of patients with metastatic triple-negative breast cancer (mTNBC). Journal of Clinical Oncology. 2016; 34(15 suppl):TPS1104-TPS.
Gandhi L, Rodriguez-Abreu D, Gadgeel S, Esteban E, Felip E, De Angelis F, et al. Pembrolizumab plus Chemotherapy in Metastatic Non-Small-Cell Lung Cancer. The New England journal of medicine. 2018; 378(22):2078-92.
Shackelford et al. The LKB1-AMPK pathway: metabolism and growth control in tumour suppression. Nat Rev Cancer 2009; 9:563-75.
Skoulidis et al. STK11/LKB1 Mutations and PD-1 Inhibitor Resistance in KRAS-Mutant Lung Adenocarcinoma. Cancer Discov. 2018 July; 8(7):822-835
Skoulidis et al. Co-occurring genomic alterations in non-small-cell lung cancer biology and therapy. Nat Rev Cancer. 2019a September; 19(9):495-509.
Skoulidis et al. Fine-Tuning Checkpoint Inhibition: Biomarkers of Response and Resistance”, 2019 ASCO Annual Meeting, J Clin Oncol 37, 2019b (suppl; abstr 102)
Sistigu A, Yamazaki T, Vacchelli E, Chaba K, Enot D P, Adam J, et al. Cancer cell-autonomous contribution of type I interferon signaling to the efficacy of chemotherapy. Nat Med. 2014; 20(1 1):1301-9.
Zitvogel L, Galluzzi L, Kepp O, Smyth M J, Kroemer G. Type I interferons in anticancer immunity. Nature reviews Immunology. 2015; 15(7):405-14.
Zhao and Xu. Targeting the LKB1 Tumor Suppressor. Curr Drug Targets. 2014 January; 15(1): 32-52.
Yang et al. New Horizons in KRAS-Mutant Lung Cancer: Dawn After Darkness. Front Oncol. 2019; 9: 953.
Jancik et al. Clinical Relevance of KRAS in Human Cancers. J Biomed Biotechnol. 2010; 2010: 150960.
Joerger & Fersht. The p53 Pathway: Origins, Inactivation in Cancer, and Emerging Therapeutic Approaches. Annu Rev Biochem. 2016 Jun. 2; 85:375-404.
Dunn et al. Pisces: an accurate and versatile variant caller for somatic and germline next-generation sequencing data. Bioinformatics. 2019 May 1; 35(9):1 579-1581.
Reva et al. Predicting the functional impact of protein mutations: application to cancer genomics. Nucleic Acids Res. 2011 Sep. 1; 39(17):e118.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. An AXL inhibitor (AXLi) and an immune checkpoint modulator (ICM) for use in the treatment of an AXL-related disease, wherein the AXL-related disease is characterised by the presence of cells having modified STK11 activity or expression; and/or
wherein the AXL-related disease is characterised by the presence of cells having increased KRAS activity or expression,
and wherein the AXLi and ICM are for administration separately, sequentially or simultaneously.

2. An AXL inhibitor (AXLi) and an immune checkpoint modulator (ICM) for use in the treatment of an AXL-related disease, said treatment comprising: identifying subjects that have previously been treated with an ICM and which did not respond to or benefit from treatment with the ICM; and, selecting thus identified subjects for treatment, wherein the AXLi and ICM are for administration separately, sequentially or simultaneously.

3. An AXL inhibitor (AXLi) and an immune checkpoint modulator (ICM) for use in the treatment of an AXL-related disease, wherein the AXL-related disease is characterised by a reduced presence of CD8⁺ cells having TCF1 activity or expression, and wherein the AXLi and ICM are for administration separately, sequentially or simultaneously.

4. The AXLi and ICM for use according to any preceding claim, wherein the use further comprises the administration of a chemotherapeutic agent and/or radiotherapy.

5. The AXLi and ICM for use according to any preceding claim, wherein the AXL-related disease is further characterised by:
i) the presence of cells having increased KRAS activity or expression;
ii) the presence of cells having decreased p53 activity or expression; and/or
iii) the presence of cells having increased AXL activity or expression.

6. The AXLi and ICM for use according to any preceding claim, wherein increased or decreased expression is assessed by:
i) determining copy number of the gene encoding STK11, KRAS, or p53 relative to a control sample; and/or
ii) determining the level of STK11, KRAS, or p53 protein or mRNA relative to a control sample.

7. The AXLi and ICM for use according to any preceding claim, wherein modified STK11 activity or expression is assessed by determining the presence or absence of a STK11 mutation and/or a STK11IP mutation.

8. The AXLi and ICM for use according to any preceding claim, wherein STK11 activity and/or expression is decreased relative to a control sample.

9. The AXLi and ICM for use according to claim 7 or claim 8, wherein the STK11 mutation or STK11IP mutation, is:
i) a mutation in the nucleotide sequence encoding STK11 or STK11IP;
ii) a mutation in a regulatory sequence controlling expression of the nucleotide sequence encoding STK11 or STK11IP;
iii) a mutation in a nucleotide encoding a protein which interacts with the transcription product of the STK11 or STK11IP gene;
iv) a mutation in the translation product of the STK11 or STK11IP gene; and/or
v) a mutation in the transcription product of the STK11 or STK11IP gene.

10. The AXLi and ICM for use according to any one of claims 7-9, wherein STK11 mutation is an inactivating mutation, and/or the STK11IP mutation is an activating mutation.

11. The AXLi and ICM for use according to any preceding claim, wherein increased or decreased activity, expression, or population is determined in a sample derived from a subject.

12. The AXLi and ICM for use according to any preceding claim, wherein increased or decreased activity, expression, or population is determined relative to a control.

13. The AXLi and ICM for use according to any preceding claim, wherein the AXL-related disease is cancer, preferably a cancer selected from the group consisting of: lung cancer, non-small-cell lung cancer, breast cancer, melanoma, mesothelioma, acute myeloid leukemia (AML), myelodysplatic syndrome (MDS), pancreas cancer, kidney cancer, urothelial carcinoma, and glioblastoma.

14. The AXLi and ICM for use according to claim 13, wherein the cancer is lung cancer, preferably non-small-cell lung cancer.

15. The AXLi and ICM for use according to any preceding claim, further comprising administering to the subject a therapeutically effective amount of an AXL inhibitor (AXLi), an immune checkpoint modulator (ICM), and/or a chemotherapeutic agent and/or radiotherapy.

16. The AXLi and ICM for use according to any preceding claim, wherein the AXL inhibitor is selected from the group consisting of:
1-(6,7-dihydro-5H-benzo[6,7]cyclohepta[1,2-c]pyridazin-3-yl)-N3-(7-(pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7-(S)-pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7-(R)-pyrrolidin-1-yl)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-(3-fluoro-4-(4-(pyrrolidin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N5-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N5-(7-(S)-pyrrolidin-1-yl-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(acetamido)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-((2R)-2-(methoxycarbonyl)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(4,4-difluoropiperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-((methoxycarbonylmethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-((2R)-2-(carboxy)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(4-(ethoxycarbonyl)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(4-(carboxy)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-((carboxymethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(4-(ethoxycarbonylmethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(4-(carboxymethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N3-((7S)-7-amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7s)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((2-methylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((propyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(dipropylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(diethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(cyclopentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((1-cyclopentylethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(2-propylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((3,3-dimethylbut-2-yl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(di(cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((5-chlorothien-2-yl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((2-carboxyphenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((3-bromophenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(dimethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(3-pentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((2,2-dimethylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(di(cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(di(bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-((bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(3-methylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(2-ethylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(but-2-enylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N3-((7S)-7-(butyl(but-2-enyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N5-((7S)-7-(t-butoxycarbonylamino)-6,7,8,9-tetrahydro-5H-benzo[7]annulene-2-yl)-1H-1,2,4-triazole-3,5-daimine;
1-(6,7-dihydro-5H-pyrido[2′,3′:6,7]cyclohepta[1,2-c]pyridazin-3-yl)-N3-((7S)-7-amino-6,7,8,9-tetrahydro-5H-benzo[7]annulene-2-yl)-1H-1,2,4-triazole-3,5-diamine;
1-(6,7-dihydro-5H-pyrido[2′,3.:6,7]cyclohepta[1,2-c]pyridazin-3-yl)-N3-((7S)-7-(dimethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(diethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(dipropylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-((methylethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N3-((7S)-7-(cyclopentylamino)-6,7,8,9-tetrahydro-5H-benzo[7]ann ulene-2-yl)-1H-1,2,4-triazole-3,5-diamine; and
1-(6,7-dihydro-5H-pyrido[2′,3′:6,7]cyclohepta[1,2-c]pyridazin-3-yl)-N3-((7S)-7-(2-butylamino)-6,7,8,9-tetrahydro-5H-benzo[7]annulene-2-yl)-1H-1,2,4-triazole-3,5-diamine;
or pharmaceutically acceptable salts thereof.

17. The AXLi and ICM for use according to any preceding claim, wherein the AXL inhibitor is:
i) Bemcentinib (BGB324/R428); or
ii) selected from the group consisting of: dubermatinib (CAS No.1341200-45-0; UNII 14D65TV20J); gilteritinib (CAS No. 1254053-43-4; UNII 66D92MGC8M); cabozantinib (CAS No. 849217-68-1; UNII 1 C39JW444G); SG17079 (CAS No. 1239875-86-5); merestinib (CAS No. 1206799-15-6; UNII 50GS5K699E); amuvatinib (CAS No. 850879-09-3; UNII SO9S6QZB4R); bosutinib (CAS No. 380843-75-4; UNII 5018V4AEZ0); glesatinib (CAS No. 936694-12-1; UNII 7Q290XD98N); foretinib (CAS No. 849217-64-7; UNII 81FH7VK1C4); and, TP0903 (CAS No. 1341200-45-0); or
iii) an anti-AXL antibody.

18. The AXLi and ICM for use according to any preceding claim, wherein the immune checkpoint modulator includes one or more immune checkpoint inhibitors (ICI), optionally wherein the immune checkpoint modulator is selected from the group consisting of:
anti-CTLA-4 antibodies, anti-PD-1 antibodies, anti-PD-L1 antibodies, anti-4-1BB antibodies, anti-OX-40 antibodies, anti-G ITR antibodies, anti-CD27 antibodies, anti-CD28 antibodies, anti-CD40 antibodies, anti-LAGS antibodies, anti-ICOS antibodies, anti-TWEAKR antibodies, anti-HVEM antibodies, anti-TIM-1 antibodies, anti-TIM-3 antibodies, anti-VISTA antibodies, and anti-TIGIT antibodies.

19. The AXLi and ICM for use according to any preceding claim, wherein the immune checkpoint modulator is selected from the group consisting of: anti-CTLA-4 antibodies, anti-PD-1 antibodies, and anti-PD-L1 antibodies.

20. The AXLi and ICM for use according to any preceding claim, wherein the immune checkpoint modulator includes, or is: pembrolizumab; ipilimumab; ipilimumab and nivolumab; ipilimumab and pembrolizumab; tremelilumab and durvalumab.

21. The AXLi and ICM for use according to any one of claims 4 to 20, wherein the chemotherapeutic agent is a chemotherapeutic agent which induces immunogenic cell death of cancer cells.

22. The AXLi and ICM for use according to any one of claims 4 to 21, wherein the chemotherapeutic agent is a chemotherapeutic agent which induces an immune response in the subject, optionally wherein the chemotherapeutic agent is a chemotherapeutic agent which induces a type I interferon response in the subject.

23. The AXLi and ICM for use according to any one of claims 4 to 22, wherein the chemotherapeutic agent is an anthracycline, optionally wherein the anthracycline is doxorubicin, daunorubicin, epirubicin, idarubicin, mitoxantrone, or valrubicin, preferably doxorubicin.

24. A method of selecting a subject for treatment with a combination of an AXL inhibitor (AXLi) and an immune checkpoint modulator (ICM), the method comprising: identifying subjects having an AXL-related disease characterised by the presence of cells having modified STK11 activity or expression; and, selecting thus identified subjects for treatment.

25. A method of selecting a subject for treatment with a combination of an AXL inhibitor (AXLi) and an immune checkpoint modulator (ICM), the method comprising: identifying subjects having an AXL-related disease characterised by the presence of cells having increased KRAS activity or expression; and, selecting thus identified subjects for treatment.

26. A method of selecting a subject for treatment with a combination of an AXL inhibitor (AXLi) and an immune checkpoint modulator (ICM), the method comprising: identifying subjects that have previously been treated with an immune checkpoint modulator (ICM) and which did not respond to or benefit from treatment with the ICM; and, selecting thus identified subjects for treatment.

27. A method of selecting a subject for treatment with a combination of an AXL inhibitor (AXLi) and an immune checkpoint modulator (ICM), the method comprising: identifying subjects having an AXL-related disease characterised by a reduced presence of CD8⁺ cells having TCF1 activity or expression; and, selecting thus identified subjects for treatment.

28. A method of increasing a population of desired T cells in a subject comprising treating the subject with an AXL inhibitor (AXLi).

29. The method of claim 28, wherein the desired T cell population is a CD8+ T cell population.

30. A method of treating an AXL-related disease in a subject with a combination of an AXL inhibitor (AXLi) and an immune checkpoint modulator (ICM), the method comprising:
(i) administering a combination of the AXLi and the ICM to the subject;
(ii) administering the AXLi to the subject, wherein the ICM has been, is, or will be, administered to the subject; or
(iii) administering the ICM to the subject, wherein the AXLi has been, is, or will be, administered to the subject;
wherein the AXL-related disease is characterised by: the presence of cells having decreased STK11 activity or expression; and/or the presence of cells having a STK11 mutation and/or a STK11IP mutation;
optionally wherein the subject has been selected for treatment using a method as defined in any one of claims 1-23.

31. A method of treating an AXL-related disease in a subject with a combination of an AXL inhibitor (AXLi) and an immune checkpoint modulator (ICM), the method comprising:
(i) administering a combination of the AXLi and the ICM to the subject;
(ii) administering the AXLi to the subject, wherein the ICM has been, is, or will be, administered to the subject; or
(iii) administering the ICM to the subject, wherein the AXLi has been, is, or will be, administered to the subject;
wherein the AXL-related disease is characterised by: the presence of cells having increased KRAS activity or expression; and/or the presence of cells having a KRAS mutation;
optionally wherein the subject has been selected for treatment using a method as defined in any one of claims 1-23.

32. The method of claim 30 or 31, wherein the subject is treated with a combination of an AXL inhibitor (AXLi), an immune checkpoint modulator (ICM), and a chemotherapeutic agent and/or radiotherapy.

33. A method of prognosing susceptibility of a subject to treatment with a combination of an AXL inhibitor (AXLi) and an immune checkpoint modulator (ICM), the method comprising:
determining: i) the presence or absence of a STK11 mutation and/or STK11I P mutation; and/or ii) the level of STK11 activity or expression in the subject or a sample derived from the subject;
wherein the presence of a STK11 mutation and/or presence of a STK11I P mutation, and/or a modified level of STK11 activity or expression is indicative of susceptibility to treatment with a combination of an AXL inhibitor (AXLi) and an immune checkpoint modulator (ICM), preferably wherein the modified level of STK11 activity or expression is a decreased level of STK11 activity or expression.

34. A method of prognosing susceptibility of a subject to treatment with a combination of an AXL inhibitor (AXLi) and an immune checkpoint modulator (ICM), the method comprising:
determining: i) the presence or absence of a KRAS mutation; and/or ii) the level of STK11 activity or expression in the subject or a sample derived from the subject;
wherein the presence of a KRAS mutation, and/or an increased level of STK11 activity or expression is indicative of susceptibility to treatment with a combination of an AXL inhibitor (AXLi) and an immune checkpoint modulator (ICM).

35. A method of treating an AXL-related disease in a subject in need of such treatment, the method comprising administering to the subject a combination of an AXL inhibitor (AXLi) and an immune checkpoint modulator (ICM), wherein the AXLi and ICM may be administered to the subject simultaneously, separately or sequentially.

36. An AXL inhibitor (AXLi) and an immune checkpoint modulator (ICM) for use in the treatment of an AXL-related disease in a subject, wherein the AXLi and ICM may be administered to the subject simultaneously, separately or sequentially.


SEQUENCE LISTING (20 sequences)

SEQ ID NO: 1 - protein, 10 aa, Artificial Sequence
  10C9 Heavy CDR1
DYNFTRYYIH

SEQ ID NO: 2 - protein, 17 aa, Artificial Sequence
  10C9 Heavy CDR2
WIYPGTGDSKYNEKFKG

SEQ ID NO: 3 - protein, 9 aa, Artificial Sequence
  10C9 Heavy CDR3
NGNYWYFDV

SEQ ID NO: 4 - protein, 16 aa, Artificial Sequence
  10C9 Light CDR1
RSSKSLLHSNGNTYLY

SEQ ID NO: 5 - protein, 7 aa, Artificial Sequence
  10C9 Light CDR2
RMSNLAS

SEQ ID NO: 6 - protein, 9 aa, Artificial Sequence
  10C9 Light CDR3
MQHREYPFT

SEQ ID NO: 7 - protein, 10 aa, Artificial Sequence
  10G5 Heavy CDR1
GYSFTDFYIN

SEQ ID NO: 8 - protein, 17 aa, Artificial Sequence
  10G5 Heavy CDR2
RIFPGGDNTYYNEKFKG

SEQ ID NO: 9 - protein, 9 aa, Artificial Sequence
  10G5 Heavy CDR3
RGLYYAMDY

SEQ ID NO: 10 - protein, 16 aa, Artificial Sequence
  10G5 Light CDR1
RSSQSLVHSNGIPYLH

SEQ ID NO: 11 - protein, 7 aa, Artificial Sequence
  10G5 Light CDR2
RVSNRFS

SEQ ID NO: 12 - protein, 9 aa, Artificial Sequence
  10G5 Light CDR3
SQGTHVPPT

SEQ ID NO: 13 - protein, 118 aa, Artificial Sequence
  hu10G5 VH(GH1)
EVQLVQSGAGLVQPGGSVRLSCAASGYSFTDFYINWVRQAPGKGLEWIARIFPGGDNTYYNEKFKGRFTLSADTSSSTAYLQLNSLRAEDTAVYYCARRGLYYAMDYWGQGTLVTVSS

SEQ ID NO: 14 - protein, 118 aa, Artificial Sequence
  hu10G5 VH(GH2)
EVQLVESGGGLVQPGGSLRLSCAASGYSFTDFYINWVRQAPGKGLEWVARIFPGGDNTYYNEKFKGRFTLSADTSKSTAYLQMNSLRAEDTAVYYCARRGLYYAMDYWGQGTLVTVSS

SEQ ID NO: 15 - protein, 112 aa, Artificial Sequence
  hu10G5 VL(GL1)]
DIQMTQSPSSLSASVGDRVTITCRSSQSLVHSNGIPYLHWYQQKPGKAPKLLIYRVSNRFSGVPSRFSGSGSGTDFTLTISSLQPEDFATYYCSQGTHVPPTFGQGTKVEIK

SEQ ID NO: 16 - protein, 112 aa, Artificial Sequence
  hu10G5 VL(GL2)]
DIQMTQSPSSLSASVGDRVTITCRSSQSLVHSNGIPYLHWYQQKPGKAPKLLIYRVSNRFSGVPSRFSGSRSGTDFTLTISSLQPEDFATYYCSQGTHVPPTFGQGTKVEIK

SEQ ID NO: 17 - protein, 448 aa, Artificial Sequence
  10G5 GH1 Heavy chain
EVQLVQSGAGLVQPGGSVRLSCAASGYSFTDFYINWVRQAPGKGLEWIARIFPGGDNTYYNEKFKGRFTLSADTSSSTAYLQLNSLRAEDTAVYYCARRGLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 448 aa, Artificial Sequence
  10G5 GH2 Heavy chain
EVQLVESGGGLVQPGGSLRLSCAASGYSFTDFYINWVRQAPGKGLEWVARIFPGGDNTYYNEKFKGRFTLSADTSKSTAYLQMNSLRAEDTAVYYCARRGLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 219 aa, Artificial Sequence
  10G5 GL1 Light chain
DIQMTQSPSSLSASVGDRVTITCRSSQSLVHSNGIPYLHWYQQKPGKAPKLLIYRVSNRFSGVPSRFSGSGSGTDFTLTISSLQPEDFATYYCSQGTHVPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 219 aa, Artificial Sequence
  10G5 GL2 Light chain
DIQMTQSPSSLSASVGDRVTITCRSSQSLVHSNGIPYLHWYQQKPGKAPKLLIYRVSNRFSGVPSRFSGSRSGTDFTLTISSLQPEDFATYYCSQGTHVPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC